SEQUENCE LISTING (10 sequences)

SEQ ID NO: 1 - DNA, 20 bp, Artificial Sequence
  Synthetic oligonucleotide
ctcaacacgggaaacctcac

SEQ ID NO: 2 - DNA, 17 bp, Artificial Sequence
  Synthetic oligonucleotide
agacaaatcgctccaac

SEQ ID NO: 3 - DNA, 20 bp, Artificial Sequence
  Synthetic oligonucleotide
tggaaggtcgaatgtgtggg

SEQ ID NO: 4 - DNA, 20 bp, Artificial Sequence
  Synthetic oligonucleotide
agcccttaagttgccttggg

SEQ ID NO: 5 - DNA, 20 bp, Artificial Sequence
  Synthetic oligonucleotide
gtatctcaccgggaggcgtt

SEQ ID NO: 6 - DNA, 20 bp, Artificial Sequence
  Synthetic oligonucleotide
cagagcgctaagctgtgatg

SEQ ID NO: 7 - DNA, 20 bp, Artificial Sequence
  Synthetic oligonucleotide
ccggatctaccttgctgctc

SEQ ID NO: 8 - DNA, 21 bp, Artificial Sequence
  Synthetic oligonucleotide
cacagcaatgcctgacaagac

SEQ ID NO: 9 - DNA, 20 bp, Artificial Sequence
  Synthetic oligonucleotide
atcgcccctacgacaagaac

SEQ ID NO: 10 - DNA, 20 bp, Artificial Sequence
  Synthetic oligonucleotide
gttgatgtgccagcggtact

(19) United States
(12) Patent Application Publication
(10) Pub. No.: US 2024/0199572 A1
(43) Pub. Date: Jun. 20, 2024

(54) COMPOUNDS, COMPOSITIONS, AND METHODS FOR TREATING TYPE 2 DIABETES AND DEMENTIA

(71) Applicants: THE BOARD OF TRUSTEES OF THE UNIVERSITY OF ILLINOIS, Chcago, IL (US); ARIZONA BOARD OF REGENTS ON BEHALF OF THE UNIVERSITY OF ARIZONA, Tucson, AZ (US)

(72) Inventors: Gregory R. THATCHER, Tucson, AZ (US); Cutler T. LEWANDOWSKI, Chicago, IL (US); Manel BEN AISSA, Chicago, IL (US); Brian LAYDEN, La Grange Park, IL (US); Yeng-Jeng SHAW, Tucson, AZ (US); Ganga Reddy VELMA, Tucson, AZ (US); Mary Jo LADU

(21) Appl. No.: 18/280,531

(22) PCT Filed: Mar. 15, 2022
(86) PCT No.: PCT/US2022/020315

§ 371 Date: Sep. 6, 2023

Related U.S. Application Data
(60) Provisional application No. 63/161,232, filed on Mar. 15, 2021.

(51) Int. Cl. C07D 333/40; A61K 31/381; A61K 31/397; A61K 31/4025; A61K 31/41; A61K 31/4245; A61K 31/428; A61K 31/4436; A61K 31/496; A61K 31/506; A61K 31/5377; A61K 31/69; A61P 3/10; A61P 25/28; C07D 409/12; C07D 413/04; C07D 413/12; C07D 417/12; C07F 5/02

(52) U.S. Cl. CPC C07D 333/40; A61K 31/381; A61K 31/397; A61K 31/4025; A61K 31/41; A61K 31/4245; A61K 31/428; A61K 31/4436; A61K 31/496; A61K 31/506; A61K 31/5377; A61K 31/69; A61P 3/10; A61P 25/28; C07D 409/12; C07D 413/04; C07D 413/12; C07D 417/12; C07F 5/025

(57) ABSTRACT

Compounds that induce expression of ATP-binding cassette transporter A1 (ABCA1) without lipogenesis are provided, as are methods of using the same to treat or prevent type 2 diabetes or dementia.

This application claims benefit of priority to U.S. Provisional Patent Application Ser. No. 63/161,232, filed Mar. 15, 2021, the content of which is incorporated herein by reference in its entirety.

This invention was made with government support under grant nos. TR002003 and R01 DK104927 awarded by the National Institutes of Health. The government has certain rights in this invention.

### INTRODUCTION

### Background

Sporadic or late-onset Alzheimer's disease (AD) and related dementia (ADRD) constitute a present and growing health crisis in the aging population. Equally, the increasing prevalence of obesity is a major risk factor for the development of chronic metabolic diseases including type 2 diabetes (T2D), which is also a risk factor for COVID19 mortality. T2D is a comorbidity with ADRD and a major risk factor for cardiovascular and cerebrovascular disease (CVD) causing cerebral infarcts that impact cognition. Specifically, impaired insulin signaling and glucose metabolism, mitochondrial dysfunction, inflammation, dyslipidemia, and impaired cholesterol mobilization may be common underlying pathogenic promoters of dementia in T2D and ADRD. Impaired insulin signaling contributes to AD pathogenesis even in patients without overt diabetes. Thus, therapeutic approaches targeting one or more underlying promoters of T2D are likely to be beneficial in treatment or prevention of CVD and ADRD.

APOE4 is the strongest genetic risk factor for ADRD and is an independent risk factor for T2D and CVD. ApoE is a component of high-density lipoprotein (HDL) and HDL-like particles that transport cholesterol and other lipids in the blood and brain, respectively. The main cholesterol transporter from cell to lipoprotein is ATP-binding cassette transporter A1 (ABCA1). ABCA1 is central to reverse cholesterol transport (RCT), a process in which cholesterol is exported from tissues to HDL particles to return to the liver, where it is metabolized or excreted. In the brain, ABCA1 adds cholesterol to HDL-like particles for distribution to various cell types or for efflux across the blood-brain barrier. Thus, ABCA1 is critical for proper maintenance of cholesterol homeostasis in the brain. Unsurprisingly, reduced ABCA1 activity or expression correlates with CVD, T2D, and AD risk.

Cell and rodent models of total or tissue-specific ABCA1 knockdown show the following: increased foam cell formation and inflammation associated with atherogenesis in CVD, impaired insulin signaling, and AD-related cognitive deficits and brain pathology. Carriers of ABCA1 loss-of-function mutations (Tangier disease) likewise are predisposed to atherosclerosis, heart disease, and impaired insulin secretion. ABCA1 variants affect plasma HDL and risk of CVD and T2D. A genome-wide association study has led to evidence of associations of common ABCA1 variants with AD. Further, a novel loss-of-function mutation in ABCA1 (N1800H) was associated with high risk of AD and CVD in a large Danish cohort. This variant affects plasma HDL and cholesterol efflux and is associated with low plasma levels of apoE.

Multiple studies have demonstrated the association between decreased ABCA1, insulin signaling, and T2D in both human patients and animal models. ABCA1 upregulation is expected positively to influence insulin signaling and inflammation in the brain and periphery and, therefore, to be of potential therapeutic utility in T2D and ADRD.

ABCA1 and apoE are under transcriptional control of nuclear receptors (NR), specifically liver X receptors (LXRs). LXR forms transcriptionally repressed heterodimeric complexes, with either a retinoid X receptor (RXR) or a peroxisome-proliferator-activated receptor (PPAR), which are derepressed (activated) by agonist binding (Ogata et al. (2009) Atherosclerosis 205:413-419; Balanarasimha et al. (2014) Biochemistry 53:2632-2643). The RXR agonist bexarotene (Bex) was reported to clear oligomeric A3, the likely proximal neurotoxic form of AR; however, Bex may cause hypertriglyceridemia and hypercholesterolemia, increasing the risk of cardiovascular and liver disease. PPARγ agonists are used clinically in treatment of T2D, and LXR agonists have shown promise in models of T2D and ADRD. (Cao et al. (2003) J. Biol. Chem. 278:1131-1136; Riddell et al. (2007) Mol. Cell. Neurosci. 34:621-628). However, lipogenesis is an inherent risk.

Sterol regulatory element-binding protein 1c (SREBP1c) plays a key role in the induction of lipogenesis by the liver. Studies with isoform-specific LXR knockout mice show that SREBP1c-mediated effects are largely controlled via LXRα, which is highly expressed in the liver, while other effects can be mediated by either LXRα or β. The nonselective LXR agonist T0901317 (T0) demonstrated reversal of insulin resistance in mice; however, this was accompanied by hyperlipidemia, as would be expected for a potent pan-LXR agonist (Cao et al. (2003) J. Biol. Chem. 278:1131-1136). In rhesus monkeys, TO positively affected ABCA1, apoE, and CSF A3 but with significant adverse effects on liver fat and triglycerides. In clinical trials, a selective LXRβ agonist upregulated LXR target genes ABCA1 and ABCG1 (Katz et al. (2009) J. Clin. Pharmacol. 49:643-649). In addition, a weak LXR agonist, designated F4 or F420, has been described, which upregulates ABCA1 without inducing lipogenic genes in the liver (Aissa et al. (2021) ACS Pharmacol. Transl. Sci. 4(1):143-154).

However, there is need for improved LXRβ agonists that upregulate ABCA1/APOE without the adverse side effect of lipogenesis. The compounds of this invention address this need in the art.

### SUMMARY OF THE INVENTION

This invention is a compound having the structure of Formula (I), Formula (II), Formula (III), Formula (IV), Formula (V), Formula (VI), Formula (VII), Formula (VIII), Formula (IX), Formula (X) or Formula (XI), as disclosed herein. In a particular aspect, the invention provides a compound having the structure of:

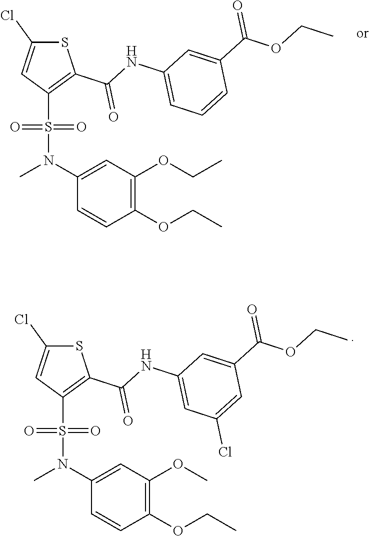 [SMILES: CCOC(=O)c1cccc(NC(=O)c2sc(Cl)cc2S(=O)(=O)N(C)c2ccc(OCC)c(OCC)c2)c1 ; CCOC(=O)c1cc(Cl)cc(NC(=O)c2sc(Cl)cc2S(=O)(=O)N(C)c2ccc(OCC)c(OC)c2)c1]

This invention also provides a pharmaceutical composition including a compound of this invention in admixture with a pharmaceutically acceptable carrier or vehicle, as well as methods of using one or more of the compounds of this invention to induce ABCA1 expression without lipogenesis and treating or preventing type 2 diabetes or dementia.

### BRIEF DESCRIPTION OF THE DRAWINGS

FIG. 1 shows results of a glucose tolerance test demonstrating blood glucose levels from t=0-120 minutes following single i.p. injection of CL2-57 at t=0. Data is presented as mean±S.D. for n=9-10 mice per group. *p<0.05, **p<0.01. Data analyzed by two-way ANOVA with post-hoc Sidak's multiple comparisons for differences between groups at each time point.

FIG. 2A-2B show densitometry analysis of liver and muscle tissue homogenates for total Akt and forms phosphorylated at Thr308 (FIG. 2A) and Ser473 (FIG. 2B) following insulin challenge test. Data is presented as mean±S.D. for n=3 mice per group. *p<0.05, ****p<0.0001. Data analyzed by t-test with Dunnett's correction for multiple comparisons.

FIG. 3A-3B show a reduction in HFD-induced mRNA expression of various proinflammatory cytokines, enzymes, and chemokines in liver (FIG. 3A) and adipose (FIG. 3B) with CL2-57 vs. vehicle treatment in HFD mice. Data presented as mean±S.D. for n=4-6 per group, with *p<0.05 by unpaired t-test.

FIG. 4 shows the percent area stained with thioflavin S (Thio-S) in various brain regions of EFAD mice treated with CL3-3 as or vehicle control.

### DETAILED DESCRIPTION OF THE INVENTION

This invention provides compounds that upregulate ABCA1 and attenuate inflammation without lipogenesis. A compound of this invention was orally administered to multiple mouse models, including mice fed a high-fat diet, resulting in restoration of insulin signaling and correction of perturbations across the metabolome. Notably, the compound of this invention exhibits LXRβ agonism with PPAR/RXR antagonism. Accordingly, the compounds of this invention find use in the prevention and treatment of inflammatory conditions such as T2D and dementia.

According to one aspect, this invention is a compound of Formula (I), or a pharmaceutically acceptable salt or prodrug thereof:

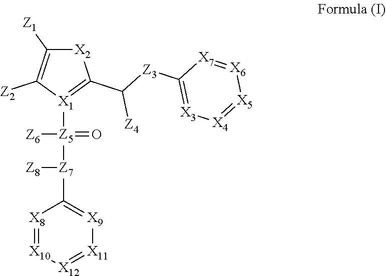

wherein,
   X₁, X₂, X₃, X₄, X₅, X₆, X₇, X₈, X₉, X₁₀, and X₁₁ are each independently selected from the group of carbon (C), nitrogen (N), sulfur (S), oxygen (O), NH, or CY₁;
   each occurrence of Y₁ is independently selected from the group consisting of hydrogen, alkyl, alkoxy, haloalkyl, hydroxyalkyl, hydroxy, cyano, halo, alkoxy, ketone, ester, carboxamide, sulfide, sulfoxide, sulfone, sulfonamide, a 5- or 6-membered monocyclic heteroaryl, and a 5- or 6-membered fused-ring;
   Z₁, Z₂, Z₃, Z₄, Z₆, and Z₈ are each independently selected from the group of hydrogen, halo, O, S, N, NH, C(═O), CH₂, hydroxy, or CY₂;
   Z₅ is S;
   Z₇ is N or C, and
   each occurrence of Y₂ is independently selected from the group consisting of hydrogen, alkyl, alkoxy, haloalkyl, hydroxyalkyl, hydroxy, cyano, and halo, with the proviso that either Z₁ is not hydrogen, or Z₈ is alkyl when Z₁ is N. Accordingly, in a particular aspect, the compound is not F420, wherein Z₁ is hydrogen, and Z₈ is hydrogen when Z₇ is N.

In another aspect, this invention is a compound of Formula (II), or a pharmaceutically acceptable salt or prodrug thereof:

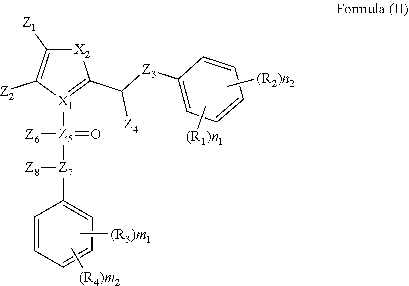

wherein,
   X₁ and X₂ are each independently selected from the group of C, N, S, O, NH, or CY₁;
   each occurrence of Y₁ is independently selected from the group consisting of hydrogen, alkyl, alkoxy, haloalkyl, hydroxyalkyl, hydroxy, cyano, and halo;
   Z₁, Z₂, Z₃, Z₄, Z₆, and Z₈ are each independently selected from the group of hydrogen, halo, O, S, N, NH, C(═O), CH₂, hydroxy, or CY₂;
   Z₅ is S;
   Z₇ is N or C; and
   each occurrence of Y₂ is independently selected from the group consisting of hydrogen, alkyl, alkoxy, haloalkyl, hydroxyalkyl, hydroxy, cyano, and halo;
   m₁ and m₂ are independently 0, 1, 2, 3, or 4;
   n₁ and n₂ are independently 0, 1, 2, 3, or 4; and
   R₁, R₂, R₃, and R₄ are each independently selected from the group of hydrogen, halo, hydroxy, nitro, C₁-C₆ alkyl, alkoxy, ketone, ester, carboxamide, sulfide, sulfoxide, sulfone, sulfonamide, C₁-C₆ fluoroalkyl, cyano, —O(C₁-C₆ alkyl), and —O(C₁-C₆fluoroalkyl),
   with the proviso that either Z₁ is not hydrogen, or Z₈ is alkyl when Z₇ is N.

In another aspect, this invention is a compound of Formula (III), or a pharmaceutically acceptable salt or prodrug thereof:

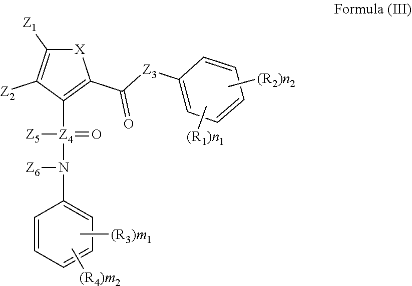

wherein,
   X is S, O, or NH;
   Z₁, Z₂, Z₃, Z₅, and Z₆ are each independently selected from the group of hydrogen, halo, O, S, N, NH, CH₂, hydroxy, or CY₁;
Z₄ is S;
each occurrence of Y₁ is independently selected from the group consisting of hydrogen, alkyl, alkoxy, haloalkyl, hydroxyalkyl, hydroxy, cyano, and halo;
m₁ and m₂ are independently 0, 1, 2, 3, or 4;
n₁ and n₂ are independently 0, 1, 2, 3, or 4; and
R₁, R₂, R₃, and R₄ are each independently selected from the group of hydrogen, halo, hydroxy, nitro, C₁-C₆ alkyl, alkoxy, ketone, ester, carboxamide, sulfide, sulfoxide, sulfone, sulfonamide, C₁-C₆ fluoroalkyl, cyano, —O(C₁-C₆ alkyl), and —O(C₁-C₆ fluoroalkyl,
with the proviso that either Z₁ is not hydrogen, or Z₆ is alkyl.

In another aspect, this invention is a compound of Formula (IV), or a pharmaceutically acceptable salt or prodrug thereof:

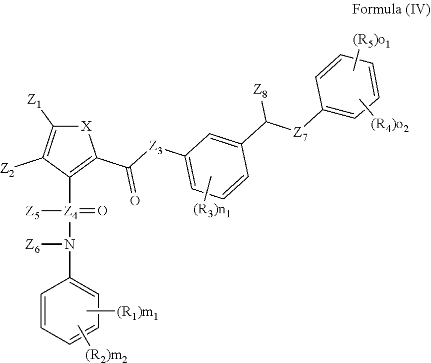

wherein,
   X is S, O, or NH;
   Z₁, Z₂, Z₃, Z₅, Z₆, Z₇ and Z₈ are each independently selected from the group of hydrogen, halo, O, S, N, NH, CH₂, hydroxy, keto (C═O) or CY₁;
   Z₄ is S;
   each occurrence of Y₁ is independently selected from the group consisting of hydrogen, alkyl, alkoxy, haloalkyl, hydroxyalkyl, hydroxy, cyano, and halo;
   m₁ and m2 are independently 0, 1, 2, 3, or 4;
   n₁ is independently 0, 1, 2, 3, or 4;
   o₁ and o₂ are independently 0, 1, 2, 3, or 4; and
   R₁, R₂, R₃, R₄ and R₅ are each independently selected from the group of hydrogen, halo, hydroxy, nitro, C₁-C₆ alkyl, alkoxy, ketone, ester, carboxamide, sulfide, sulfoxide, sulfone, sulfonamide, C₁-C₆ fluoroalkyl, cyano, —O(C₁-C₆ alkyl), and —O(C₁-C₆ fluoroalkyl),
   with the proviso that either Z₁ is not hydrogen, or Z₆ is alkyl.

In another aspect, this invention is a compound of Formula (V), or a pharmaceutically acceptable salt or prodrug thereof:

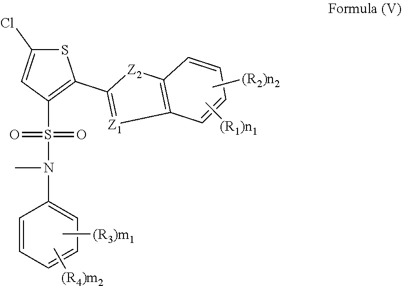

wherein,
   Z₁ and Z₂ are each independently selected from the group of N, S, O, NH, CH, or NY₁;
   each occurrence of Y₁ is independently selected from the group consisting of alkyl, haloalkyl, and hydroxyalkyl;
   m₁ and m₂ are independently 0, 1, 2, 3, or 4;
   n₁ and n₂ are independently 0, 1, 2, 3, or 4; and
   R₁, R₂, R₃ and R₄ are each independently selected from the group of hydrogen, halo, hydroxy, nitro, C₁-C₆ alkyl, alkoxy, ketone, ester, carboxamide, sulfide, sulfoxide, sulfone, sulfonamide, C₁-C₆ fluoroalkyl, cyano, —O(C₁-C₆ alkyl), and —O (C₁-C₆ fluoroalkyl).

In another aspect, this invention is a compound of Formula (VI), or a pharmaceutically acceptable salt or prodrug thereof:

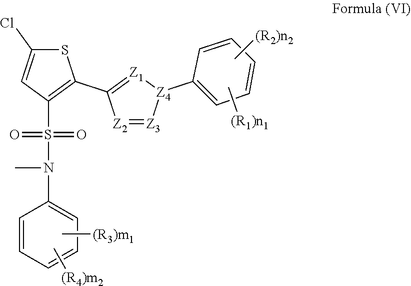

wherein,
   Z₁, Z₂, Z₃, and Z₄ are each independently selected from the group of N, S, O, NH, CH, or NY₁;
   each occurrence of Y₁ is independently selected from the group consisting of alkyl, haloalkyl, and hydroxyalkyl;
   m₁ and m₂ are independently 0, 1, 2, 3, or 4;
   n₁ and n₂ are independently 0, 1, 2, 3, or 4; and
   R₁, R₂, R₃ and R₄ are each independently selected from the group of hydrogen, halo, hydroxy, nitro, C₁-C₆ alkyl, alkoxy, ketone, ester, carboxamide, sulfide, sulfoxide, sulfone, sulfonamide, C₁-C₆ fluoroalkyl, cyano, —O(C₁-C₆ alkyl), or —O(C₁-C₆ fluoroalkyl).

In another aspect, this invention is a compound of Formula (VII), or a pharmaceutically acceptable salt or prodrug thereof:

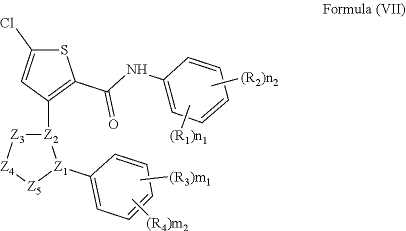

wherein,
   Z₁, Z₂, Z₃, Z₄ and Z₅ are each independently selected from the group of N, S, O, C(═O), NH, CH, or NY₁;
   each occurrence of Y₁ is independently selected from the group consisting of alkyl, haloalkyl, and hydroxyalkyl;
   m₁ and m₂ are independently 0, 1, 2, 3, or 4;
   n₁ and n₂ are independently 0, 1, 2, 3, or 4; and
   R₁, R₂, R₃ and R₄ are each independently selected from the group of hydrogen, halo, hydroxy, nitro, C₁-C₆ alkyl, alkoxy, ketone, ester, carboxamide, sulfide, sulfoxide, sulfone, sulfonamide, C₁-C₆ fluoroalkyl, cyano, —O(C₁-C₆ alkyl), or —O(C₁-C₆ fluoroalkyl).

In another aspect, this invention is a compound of Formula (VIII), or a pharmaceutically acceptable salt or prodrug thereof:

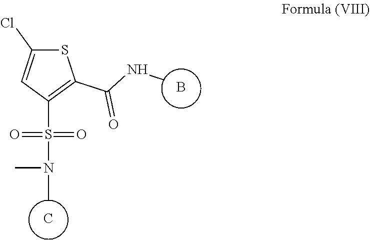

wherein,
   Ring B is selected from

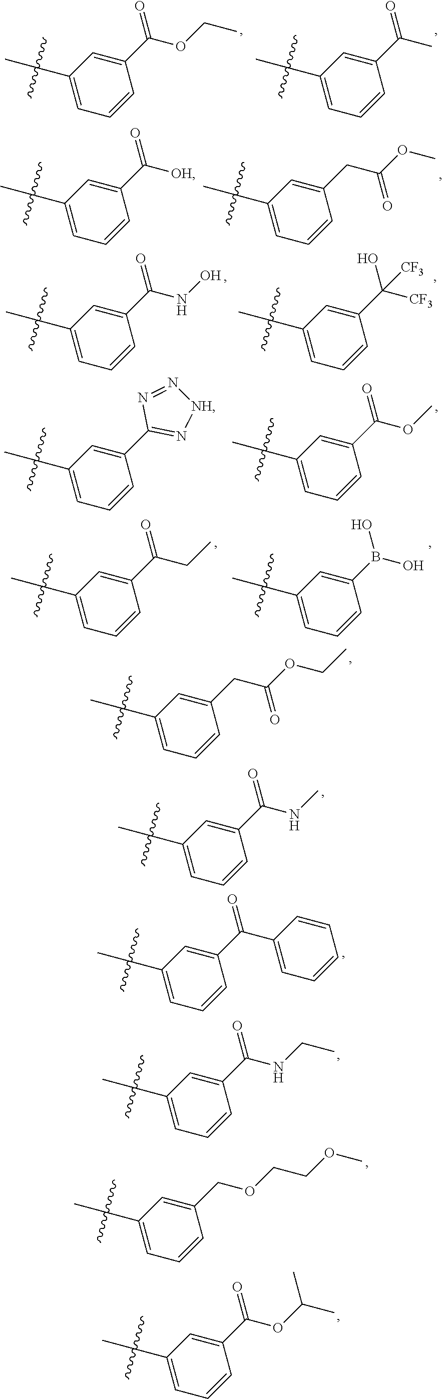

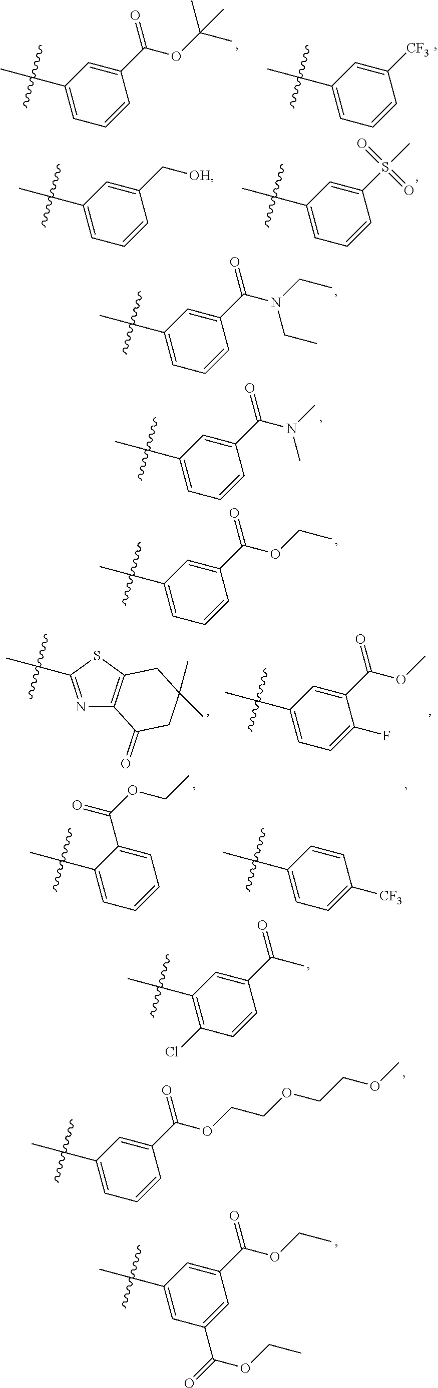

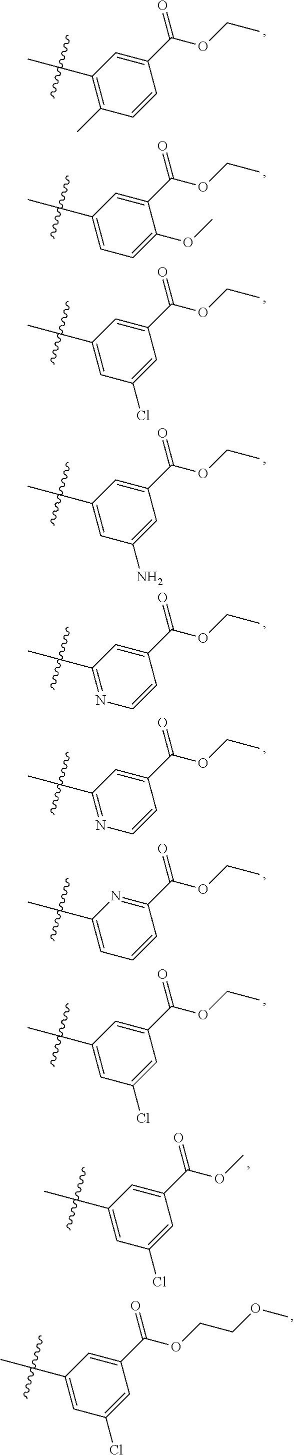

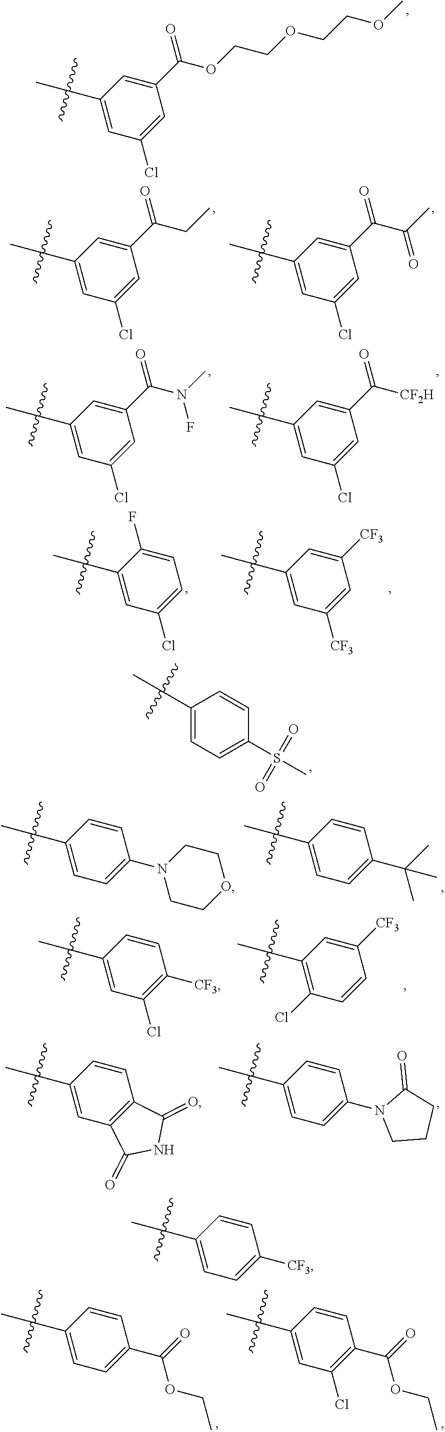

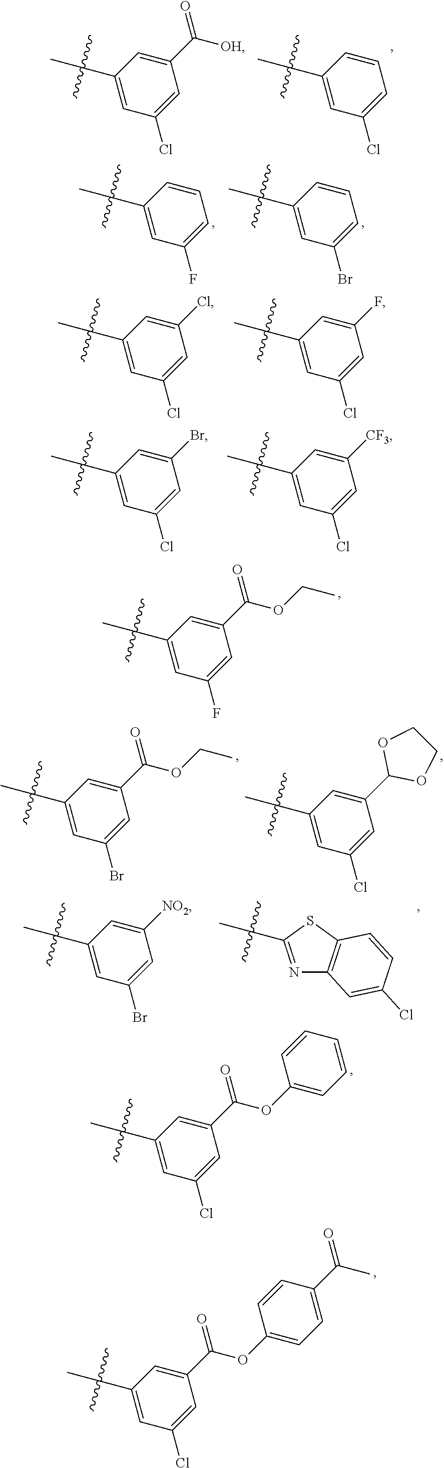

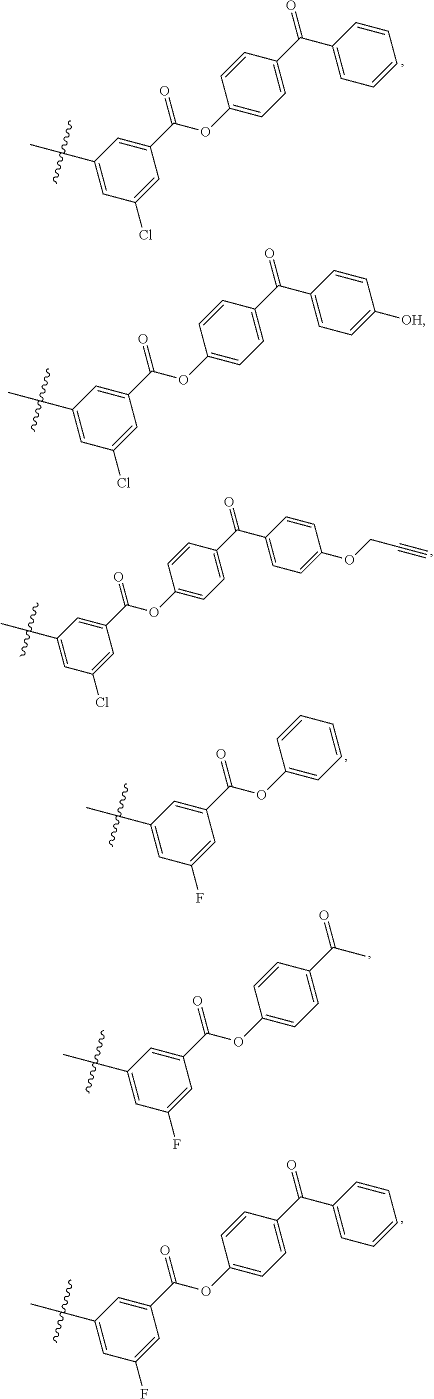 [SMILES: *c1cc(Cl)cc(C(=O)Oc2ccc(C(=O)c3ccccc3)cc2)c1 ; *c1cc(Cl)cc(C(=O)Oc2ccc(C(=O)c3ccc(O)cc3)cc2)c1 ; *c1cc(Cl)cc(C(=O)Oc2ccc(C(=O)c3ccc(OCC#C)cc3)cc2)c1 ; *c1cc(F)cc(C(=O)Oc2ccccc2)c1 ; *c1cc(F)cc(C(=O)Oc2ccc(C(C)=O)cc2)c1 ; *c1cc(F)cc(C(=O)Oc2ccc(C(=O)c3ccccc3)cc2)c1]

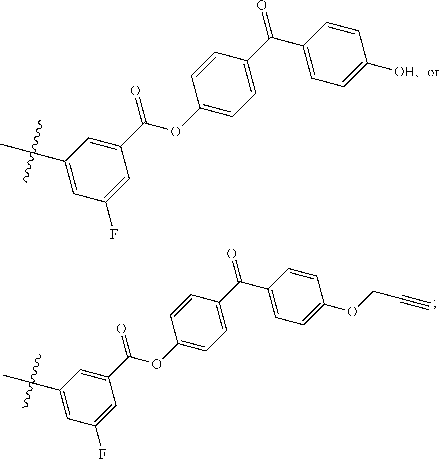 [SMILES: *c1cc(F)cc(C(=O)Oc2ccc(C(=O)c3ccc(O)cc3)cc2)c1 ; *c1cc(F)cc(C(=O)Oc2ccc(C(=O)c3ccc(OCC#C)cc3)cc2)c1]

and
   Ring C is selected from

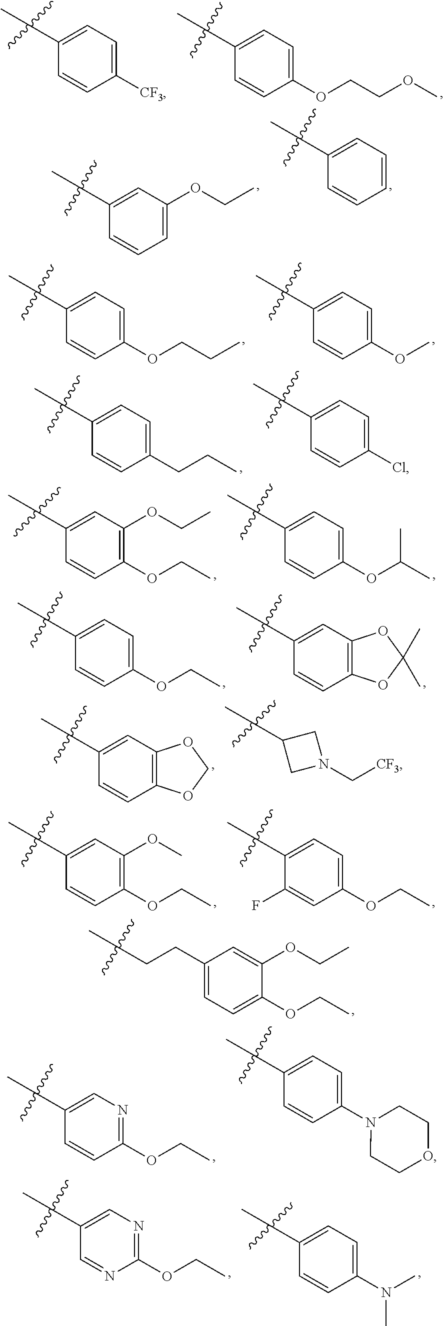

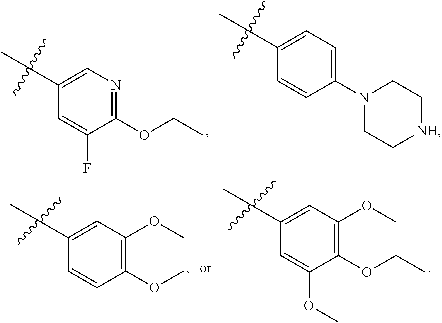 [SMILES: *c1cnc(OCC)c(F)c1 ; *c1ccc(N2CCNCC2)cc1 ; *c1ccc(OC)c(OC)c1 ; *c1cc(OC)c(OCC)c(OC)c1]

In another aspect, this invention is a compound of Formula (IX), or a pharmaceutically acceptable salt or prodrug thereof:

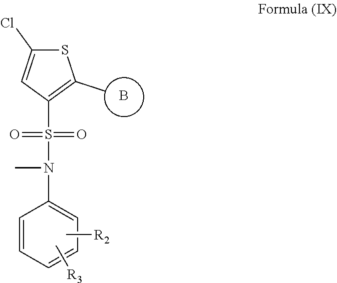

wherein,
   Ring B is selected from

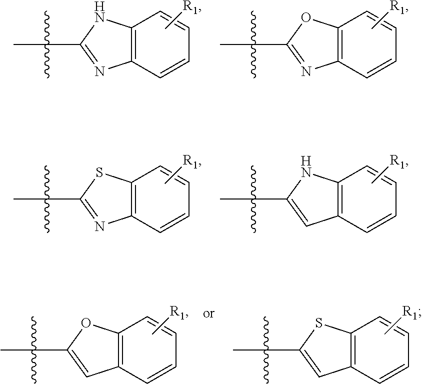

R₁ is hydrogen, halo, alkyl, alkoxy, CO₂Me, CO₂Et, cyano, or OCF₃; and
R₂ and R₃ are each independently selected from the group of alkoxy, alkyl, halo, or cyano.

In another aspect, this invention is a compound of Formula (X), or a pharmaceutically acceptable salt or prodrug thereof:

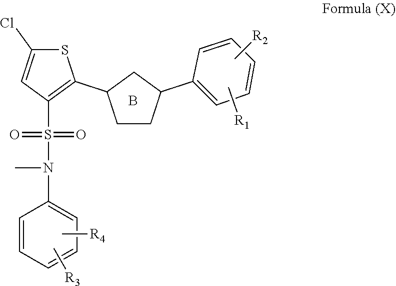

wherein
   Ring B is selected from

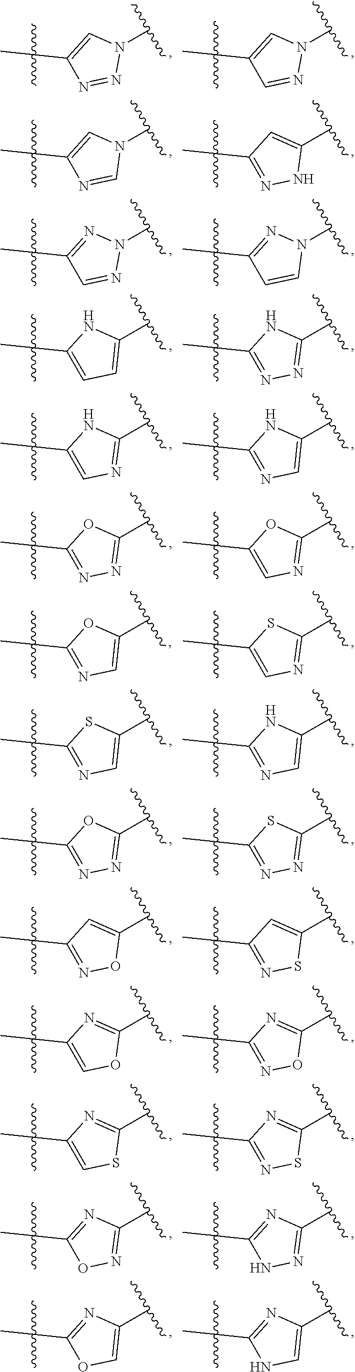

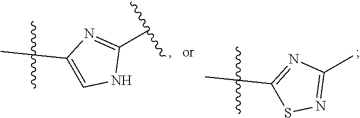

R₁ and R₂ are each independently selected from the group of hydrogen, halo, alkyl, alkoxy, CO₂Me, CO₂Et, cyano, or OCF₃; and
R₃ and R₄ are each independently selected from the group of alkoxy, alkyl, halo, or cyano.

In another aspect, this invention is a compound of Formula (XI), or a pharmaceutically acceptable salt or prodrug thereof:

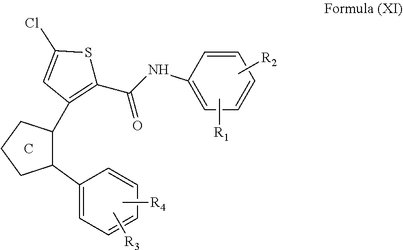

wherein,
   Ring C is selected from

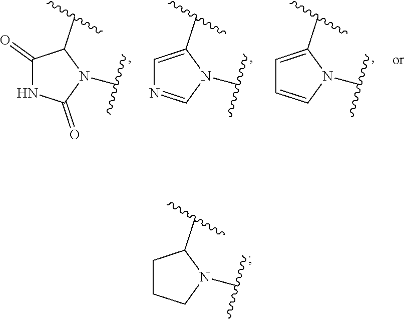

R₁ and R₂ are each independently selected from the group of hydrogen, halo, alkyl, alkoxy, CO₂Me, CO₂Et, cyano, or OCF₃; and
R₃ and R₄ are each independently selected from the group of alkoxy, alkyl, halo, or cyano.

The term “halo” or “halogen” refers to a fluorine (fluoro, —F), chlorine (chloro, —Cl), bromine (bromo, —Br), or iodine (iodo, —I) group. In certain aspects, “halo” or “halogen” refers to a Cl or F group. “Cyano” refers to a —CN group, “hydroxy” refers to a —OH group, and “nitro” refers to a —NO₂ group. “O” as a substituent refers to oxo (═O).

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH (CH₂CH₃) CH₂—.

In certain aspects, the groups disclosed herein are delineated by a specified number of carbon atoms, e.g., C₁-C₆. The numerical range “1 to 6” or “1-6” refers to each integer in the given range, e.g., “1 to 6 carbon atoms” means that a group may have 1 carbon atom, 2 carbon atoms, 3 carbon atoms, etc., up to and including 6 carbon atoms. By way of example, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus, C₁-C₄ alkyl includes C₁-C₂ alkyl and C₁-C₃ alkyl.

“Alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “haloalkyl” as used herein, means at least one halogen, as defined herein, is appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl. In certain aspects, each “haloalkyl” is a fluoroalkyl, for example, a polyfluoroalkyl such as a substantially perfluorinated alkyl.

As used herein, the term “hydroxyalkyl” means an alkyl group, as defined herein, with one or more (e.g., 1, 2 or 3) hydroxy substituents.

The term “ketone” as used herein refers to the structure —C(═O)—Ra, wherein Ra may be alkyl (e.g., —C(═O)—CH₃) or aryl.

The term “ester” as used herein refers to the structure —C(═O)—O—R_{c}, where R_{c} may be alkyl, alkenyl, alkynyl, cycloalkyl, heterocyclyl, or aryl.

“Carboxamide” refers to a group having the structure —C(═O)—N(R_{d}Rₑ), where R_{d} and Rₑ independently represent hydrogen or alkyl, alkenyl, alkynyl, cycloalkyl, heterocyclyl, or aryl groups.

The term “sulfide” refers to a group having the structure —S—Rₑ, where R_{f} may be alkyl, alkenyl, alkynyl, cycloalkyl, heterocyclyl, or aryl. Examples of sulfide groups include a methylthio group, an ethylthio group, a t-butylthio group, and a tert-butylthio group.

“Sulfinyl” or “sulfoxide” refers to the —S(═O)—R_{g} group, wherein for “sulfinyl” R_{g} is hydrogen, and for “sulfoxide” R_{g} may be alkyl, alkenyl, alkynyl, cycloalkyl, heterocyclyl or aryl.

“Sulfone” or “sulfonyl” refers to a —S(═O)₂—Rₕ group, wherein Rₕ is selected from alkyl, alkenyl, alkynyl, cycloalkyl, heterocyclyl or aryl.

“Sulfonamide” refers to a group having the structure —S(═O)₂—N(RᵢRⱼ), where R₁ and Rⱼ independently represent hydrogen or alkyl, alkenyl, alkynyl, cycloalkyl, heterocyclyl, or aryl groups.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls), and non-aromatics (e.g., cycloalkyls and non-aromatic heterocycles). Rings can be monocyclic or fused-ring, i.e., polycyclic. Rings can be optionally substituted.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

“Ring system substituent” means a substituent attached to an aromatic or non-aromatic ring system, which, for example, replaces an available hydrogen on the ring system. Ring system substituents may be the same or different, each being independently selected from the group of —C(═NH) (NH₂), —NHC(═NH) (NH₂), alkyl, alkenyl, alkynyl, alkoxy, acyl, alkylcarbonylamino, carboxy, carboxyalkyl, alkoxycarbonyl, alkoxycarbonylalkyl, cyano, nitro, alkylthio, halo, haloalkyl, haloalkoxy, amino, alkylamino, dialkylamino, aminocarbonyl, alkyl aminocarbonyl, dialkylaminocarbonyl, alkyl sulfonyl, cycloalkylsulfonyl, alkylsulfonylamino, alkylaminosulfonyl, haloalkylamino, oxo, hydroxy, hydroxyalkyl, hydroxyalkyloxy, hydroxyalkyloxyalkyl, alkoxyalkyloxyalkyl, aryl, heteroaryl, cycloalkyl, cycloalkylamino, cycloalkyloxy, heteroaralkyloxy, aminoalkyl, aminoalkyloxy, alkoxyalkyl, alkoxyalkylcarbonyl, alkoxyalkyloxy, haloalkoxyalkyl, aryloxyalkyl, heteroaryloxyalkyl, heterocycloalkyl, heterocycloalkyloxyalkyl, heterocycloalkylalkyl, heterocycloalkylalkyloxy, or heterocycloalkyloxy. “Ring system substituent” may also mean a single moiety that simultaneously replaces two available hydrogens on two adjacent carbon atoms (one H on each carbon) on a ring system. Examples of such moieties are methylenedioxy, ethylenedioxy, —C(CH₃)₂— and the like.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic akyls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2 (3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain aspects, the bicyclic aryl is (i) naphthyl or (ii) a phenyl ring fused to either a 5- or 6-membered monocyclic cycloalkyl, a 5- or 6-membered monocyclic cycloalkenyl, or a 5- or 6-membered monocyclic heterocyclyl, wherein the fused cycloalkyl, cycloalkenyl, and heterocyclyl groups are optionally substituted. In certain embodiments of the disclosure, the aryl group is phenyl or naphthyl. In certain other embodiments, the aryl group is phenyl.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain aspects, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. In certain aspects, the fused bicyclic cycloalkyl is a 5- or 6-membered monocyclic cycloalkyl ring fused to either a phenyl ring, a 5- or 6-membered monocyclic cycloalkyl, a 5- or 6-membered monocyclic cycloalkenyl, a 5- or 6-membered monocyclic heterocyclyl, or a 5- or 6-membered monocyclic heteroaryl, wherein the fused bicyclic cycloalkyl is optionally substituted.

The term “heteroaryl,” as used herein, means a monocyclic heteroaryl or a bicyclic ring system containing at least one heteroaromatic ring. The monocyclic heteroaryl can be a 5- or 6-membered ring. The 5-membered ring consists of two double bonds and one, two, three or four nitrogen atoms and optionally one oxygen or sulfur atom. The 6-membered ring consists of three double bonds and one, two, three or four nitrogen atoms. The 5- or 6-membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a phenyl ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. In certain aspects, the fused bicyclic heteroaryl is a 5- or 6-membered monocyclic heteroaryl ring fused to either a phenyl ring, a 5- or 6-membered monocyclic cycloalkyl, a 5- or 6-membered monocyclic cycloalkenyl, a 5- or 6-membered monocyclic heterocyclyl, or a 5- or 6-membered monocyclic heteroaryl, wherein the fused cycloalkyl, cycloalkenyl, and heterocyclyl groups are optionally substituted.

The term “heterocyclyl” as used herein, means a monocyclic heterocycle or a bicyclic heterocycle. The monocyclic heterocycle is a 5- or 6-membered ring containing at least one heteroatom independently selected from the group consisting of O, N, and S where the ring is saturated or unsaturated, but not aromatic. The 5-membered ring can contain zero or one double bond and one, two or three heteroatoms selected from the group consisting of 0, N and S. The 6-membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted. In certain embodiments, the bicyclic heterocyclyl is a 5- or 6-membered monocyclic heterocyclyl ring fused to phenyl ring, a 5- or 6-membered monocyclic cycloalkyl, a 5- or 6-membered monocyclic cycloalkenyl, a 5- or 6-membered monocyclic heterocyclyl, or a 5- or 6-membered monocyclic heteroaryl, wherein the bicyclic heterocyclyl is optionally substituted.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆ alkoxycarbonyloxy and —OC(═O)—C₁-C₆ alkyl indicate the same functionality; similarly, arylalkyl and alkylaryl indicate the same functionality.

In all structural formulas used herein, it is understood that all carbon valences not shown here are satisfied by the groups illustrated and by hydrogen atoms.

The term “saturated” as used herein means the referenced chemical structure does not contain any multiple carbon-carbon bonds. For example, a saturated cycloalkyl group as defined herein includes cyclohexyl, cyclopropyl, and the like.

The term “unsaturated” as used herein means the referenced chemical structure contains at least one multiple carbon-carbon bond but is not aromatic. For example, an unsaturated cycloalkyl group as defined herein includes cyclohexenyl, cyclopentenyl, cyclohexadienyl, and the like.

In all structural formulas used hereinafter, it is understood that all carbon valences not shown here are satisfied by the groups illustrated and/or by hydrogen atoms.

The compounds of this invention include the compounds themselves as well as tautomers, salts, solvates, amides, and/or esters thereof.

The term “tautomer” as used herein refers to constitutional isomers that can readily change into one another by migration of an atom or group (e.g., H) so that they can exist together in equilibrium. The compounds may also exist in several tautomeric forms including the enol form, the keto form, and mixtures thereof. Accordingly, the chemical structures depicted herein encompass all possible tautomeric forms of the illustrated compounds.

“Salt” refers to a salt of a compound, which possesses the desired activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Solvate” means a compound formed by solvation (the combination of solvent molecules with molecules or ions of the solute), or an aggregate that consists of a solute ion or molecule, i.e., a compound of the present invention, with one or more solvent molecules. Examples of acceptable solvents include water, ethanol, isopropanol, ethyl acetate, acetic acid, and ethanolamine. When water is the solvent, the corresponding solvate is “hydrate.”

The compounds of the present invention can be synthesized as described herein or according to suitable procedures known in the art. When synthesized, the compounds be prepared using suitable starting materials through a synthetic route according to known approaches for the synthesis of similar compounds. Synthetic chemistry transformations and protecting group methodologies (protection and deprotection) useful in synthesizing the starting materials and intermediates are known in the art, including, for example, Larock, Comprehensive Organic Transformations (2^{nd} Ed., VCH Publishers 1999); Wuts & Greene, Greene's Protective Groups in Organic Synthesis (4^{th} Ed., John Wiley and Sons 2007); Fieser & Fieser, Fieser and Fieser's Reagents for Organic Synthesis (John Wiley and Sons 1994); and Paquette, ed., Encyclopedia of Reagents for Organic Synthesis (2^{nd} ed., John Wiley and Sons 2009) and subsequent editions thereof.

Certain compounds of this invention may contain a non-aromatic double bond and one or more asymmetric centers. Thus, the chemical structures depicted herein encompass all possible stereoisomers (i.e., enantiomers, diastereomers, and cis- or trans-isomers) and stereoisomeric mixtures. Stereoisomeric mixtures can be resolved into their component enantiomers, diastereomers, or cis-/trans-isomers using separation techniques or chiral synthesis techniques well known to the skilled artisan.

This invention provides compounds of Formula (I), Formula (II), Formula (III), Formula (IV), Formula (V), Formula (VI), Formula (VII), or Formula (VIII), Formula (IX), Formula (X), or Formula (XI), or a pharmaceutically acceptable salts thereof. Representative compounds of the invention include, but are not limited to:

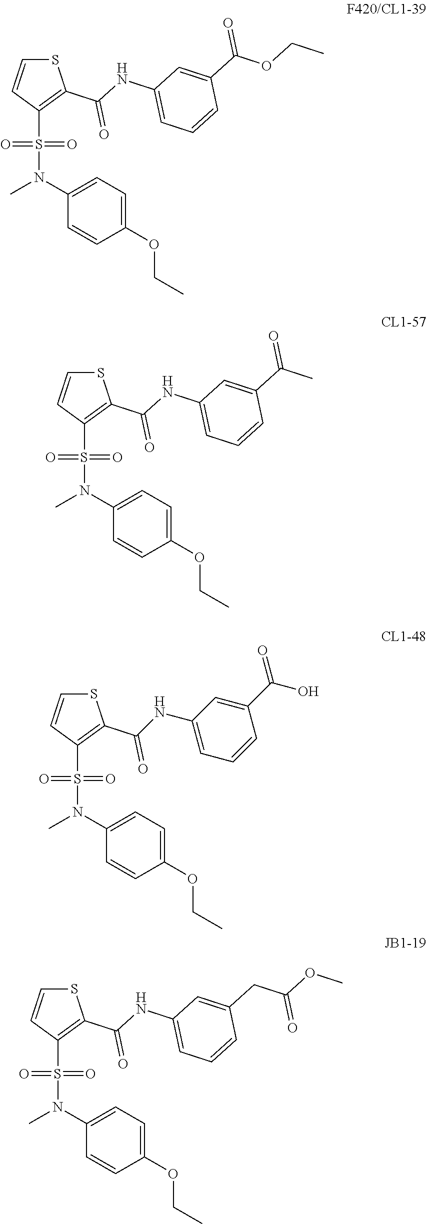 [SMILES: CCOC(=O)c1cccc(NC(=O)c2sccc2S(=O)(=O)N(C)c2ccc(OCC)cc2)c1 ; CCOc1ccc(N(C)S(=O)(=O)c2ccsc2C(=O)Nc2cccc(C(C)=O)c2)cc1 ; CCOc1ccc(N(C)S(=O)(=O)c2ccsc2C(=O)Nc2cccc(C(=O)O)c2)cc1 ; CCOc1ccc(N(C)S(=O)(=O)c2ccsc2C(=O)Nc2cccc(CC(=O)OC)c2)cc1]

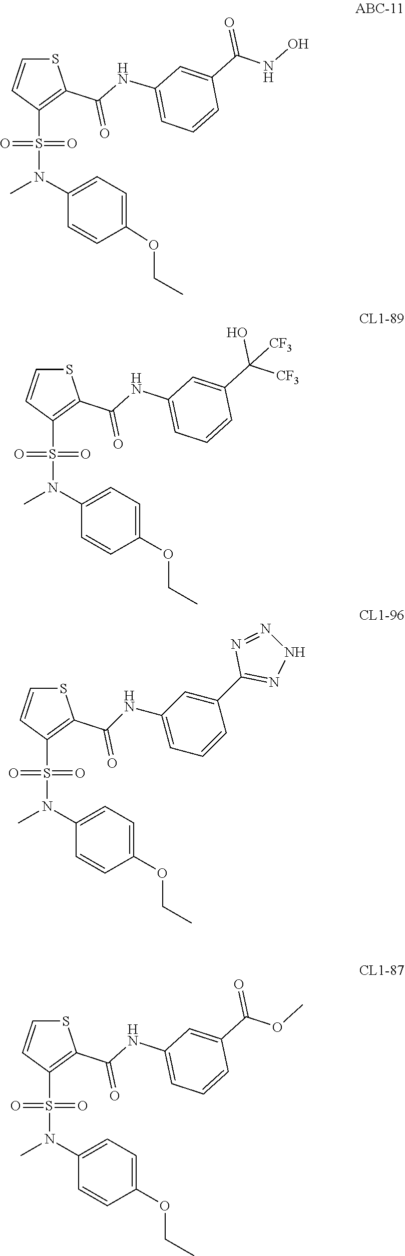 [SMILES: CCOc1ccc(N(C)S(=O)(=O)c2ccsc2C(=O)Nc2cccc(C(=O)NO)c2)cc1 ; CCOc1ccc(N(C)S(=O)(=O)c2ccsc2C(=O)Nc2cccc(C(O)(C(F)(F)F)C(F)(F)F)c2)cc1 ; CCOc1ccc(N(C)S(=O)(=O)c2ccsc2C(=O)Nc2cccc(-c3nn[nH]n3)c2)cc1 ; CCOc1ccc(N(C)S(=O)(=O)c2ccsc2C(=O)Nc2cccc(C(=O)OC)c2)cc1]

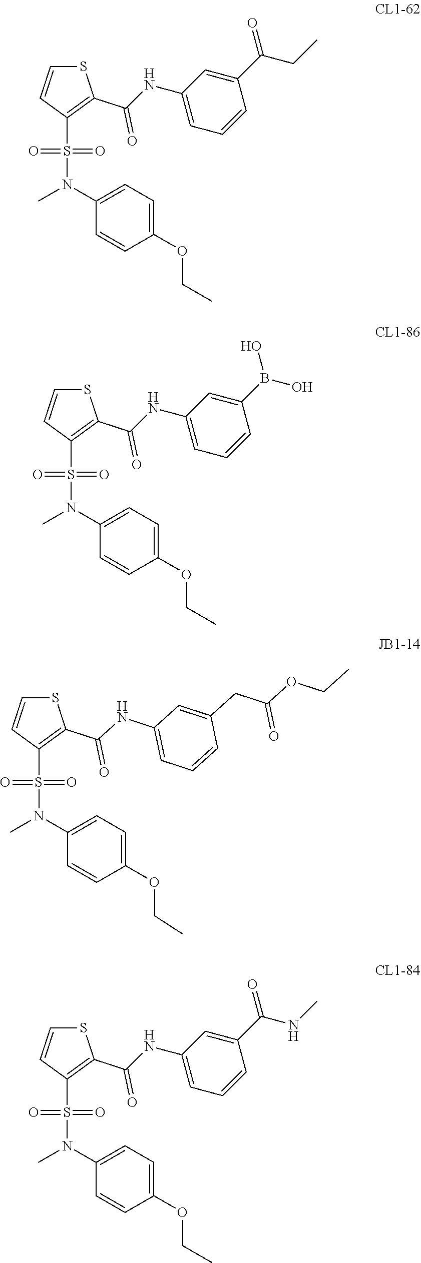 [SMILES: CCOc1ccc(N(C)S(=O)(=O)c2ccsc2C(=O)Nc2cccc(C(=O)CC)c2)cc1 ; CCOc1ccc(N(C)S(=O)(=O)c2ccsc2C(=O)Nc2cccc(B(O)O)c2)cc1 ; CCOC(=O)Cc1cccc(NC(=O)c2sccc2S(=O)(=O)N(C)c2ccc(OCC)cc2)c1 ; CCOc1ccc(N(C)S(=O)(=O)c2ccsc2C(=O)Nc2cccc(C(=O)NC)c2)cc1]

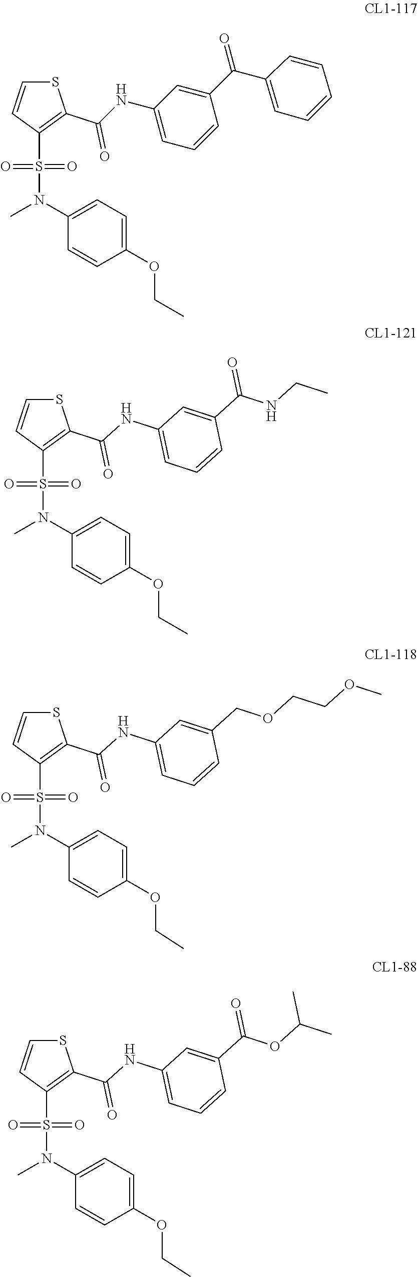 [SMILES: CCOc1ccc(N(C)S(=O)(=O)c2ccsc2C(=O)Nc2cccc(C(=O)c3ccccc3)c2)cc1 ; CCNC(=O)c1cccc(NC(=O)c2sccc2S(=O)(=O)N(C)c2ccc(OCC)cc2)c1 ; CCOc1ccc(N(C)S(=O)(=O)c2ccsc2C(=O)Nc2cccc(COCCOC)c2)cc1 ; CCOc1ccc(N(C)S(=O)(=O)c2ccsc2C(=O)Nc2cccc(C(=O)OC(C)C)c2)cc1]

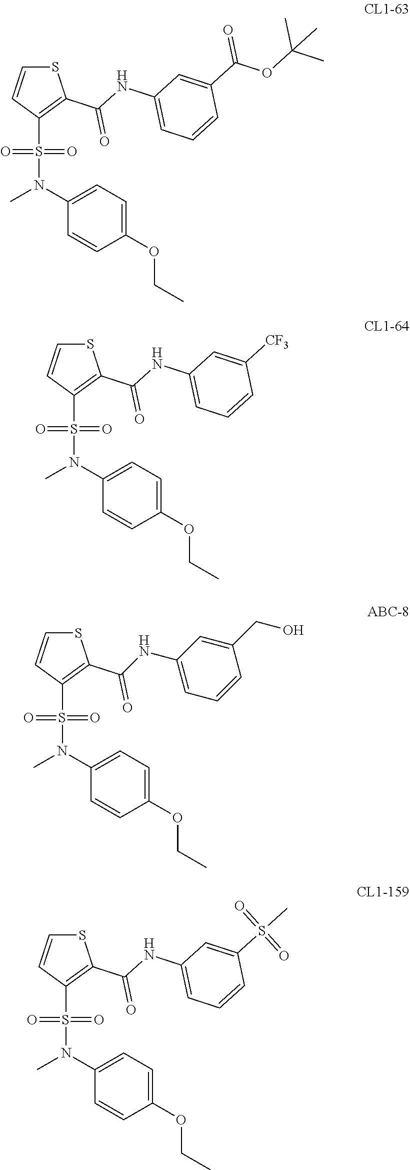

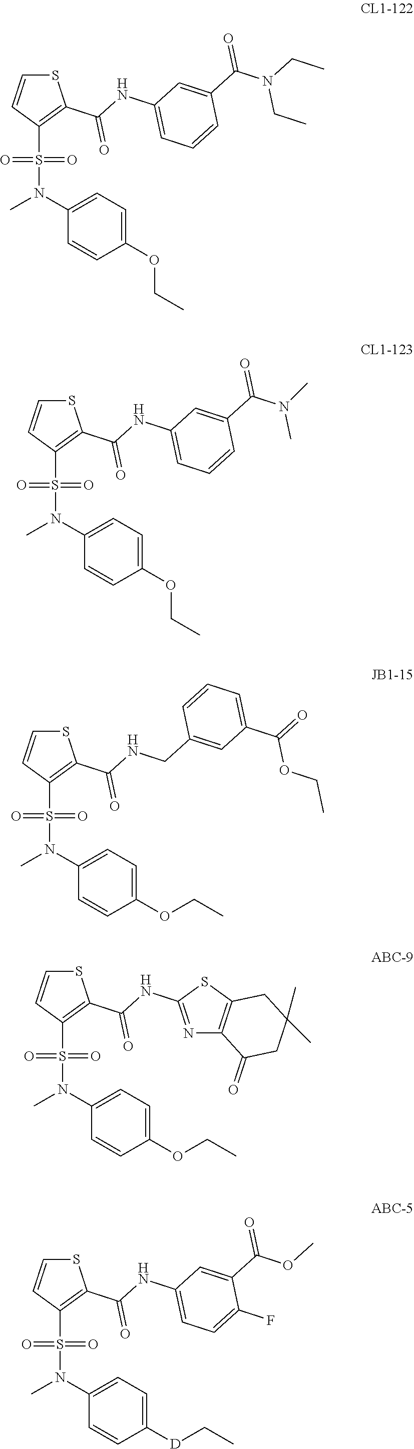

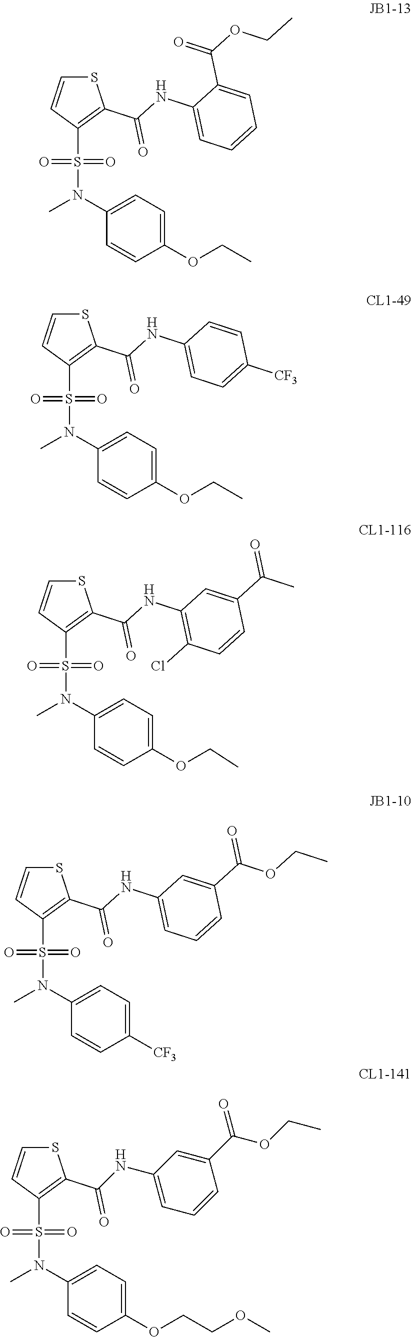 [SMILES: CCOC(=O)c1ccccc1NC(=O)c1sccc1S(=O)(=O)N(C)c1ccc(OCC)cc1 ; CCOc1ccc(N(C)S(=O)(=O)c2ccsc2C(=O)Nc2ccc(C(F)(F)F)cc2)cc1 ; CCOc1ccc(N(C)S(=O)(=O)c2ccsc2C(=O)Nc2cc(C(C)=O)ccc2Cl)cc1 ; CCOC(=O)c1cccc(NC(=O)c2sccc2S(=O)(=O)N(C)c2ccc(C(F)(F)F)cc2)c1 ; CCOC(=O)c1cccc(NC(=O)c2sccc2S(=O)(=O)N(C)c2ccc(OCCOC)cc2)c1]

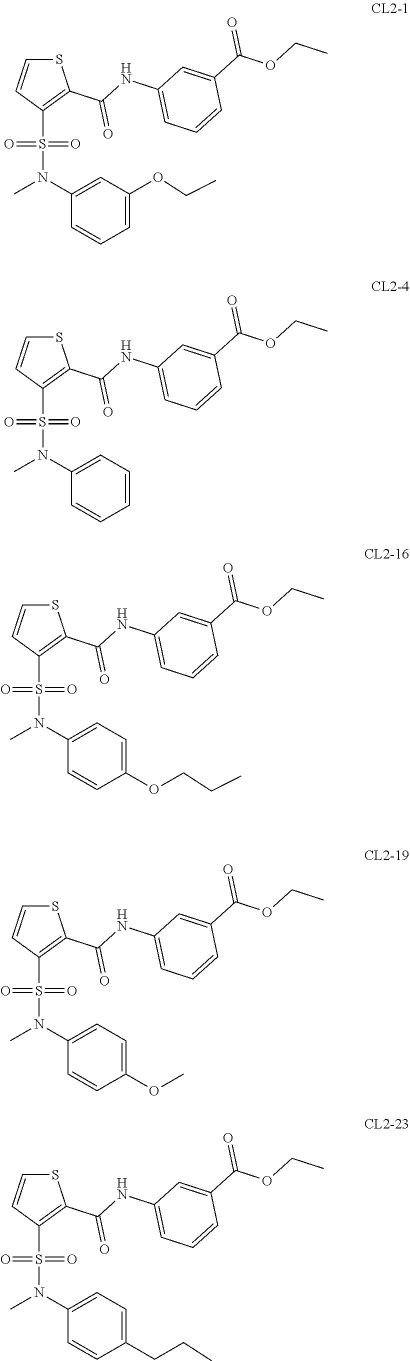 [SMILES: CCOC(=O)c1cccc(NC(=O)c2sccc2S(=O)(=O)N(C)c2cccc(OCC)c2)c1 ; CCOC(=O)c1cccc(NC(=O)c2sccc2S(=O)(=O)N(C)c2ccccc2)c1 ; CCCOc1ccc(N(C)S(=O)(=O)c2ccsc2C(=O)Nc2cccc(C(=O)OCC)c2)cc1 ; CCOC(=O)c1cccc(NC(=O)c2sccc2S(=O)(=O)N(C)c2ccc(OC)cc2)c1 ; CCCc1ccc(N(C)S(=O)(=O)c2ccsc2C(=O)Nc2cccc(C(=O)OCC)c2)cc1]

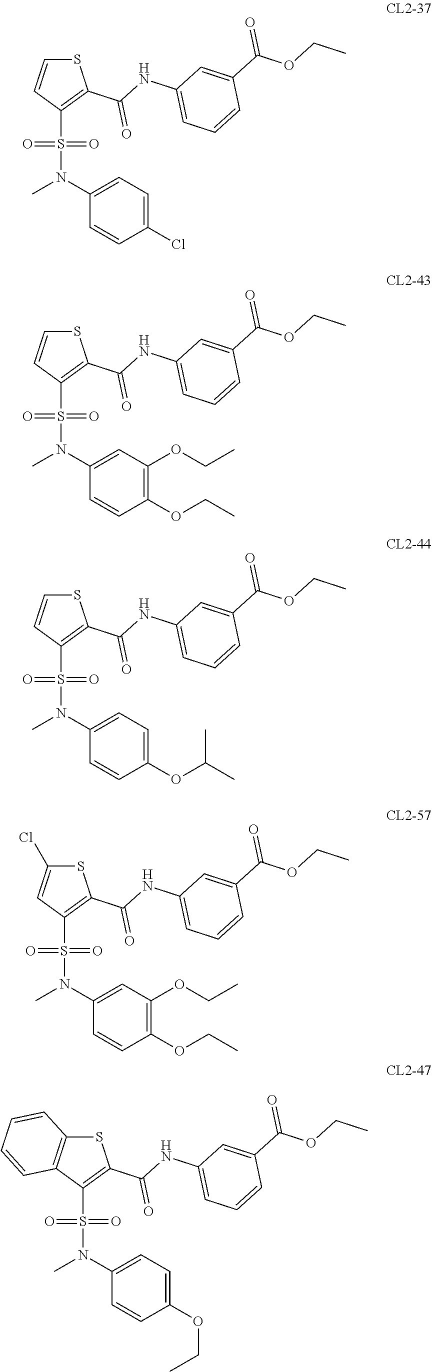

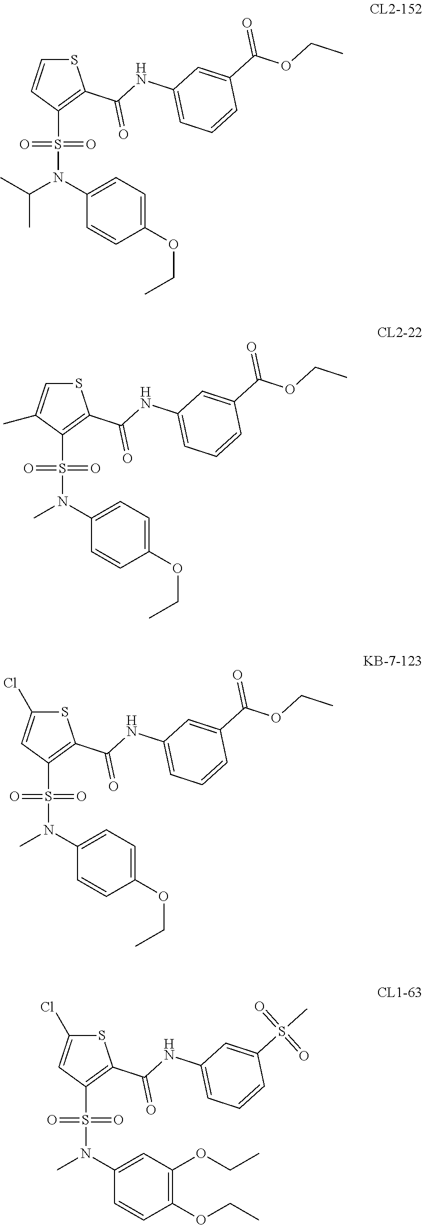

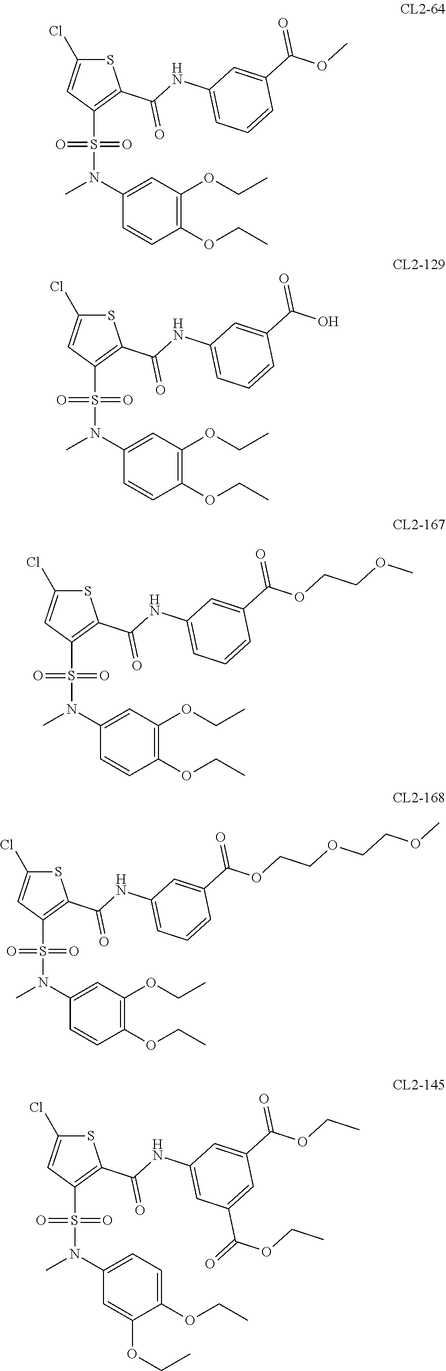

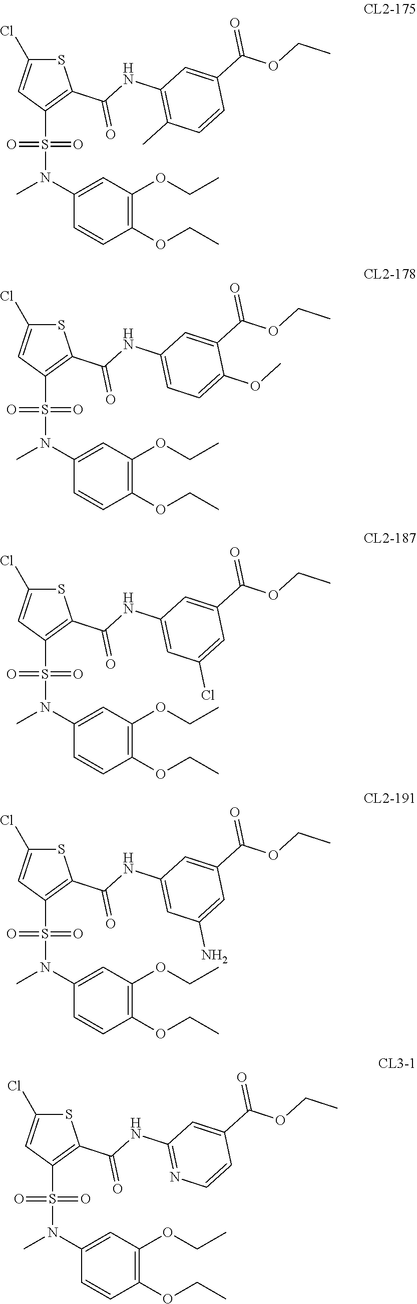

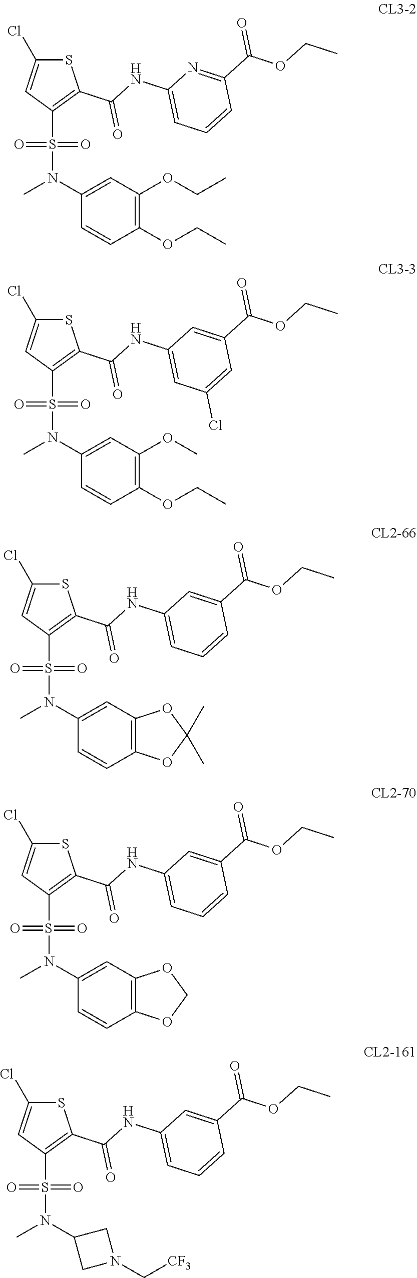

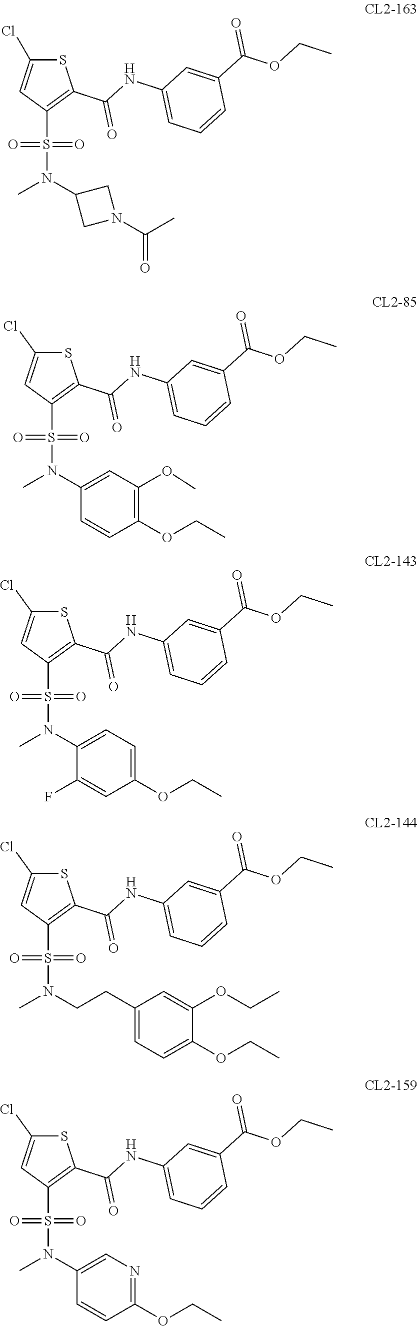

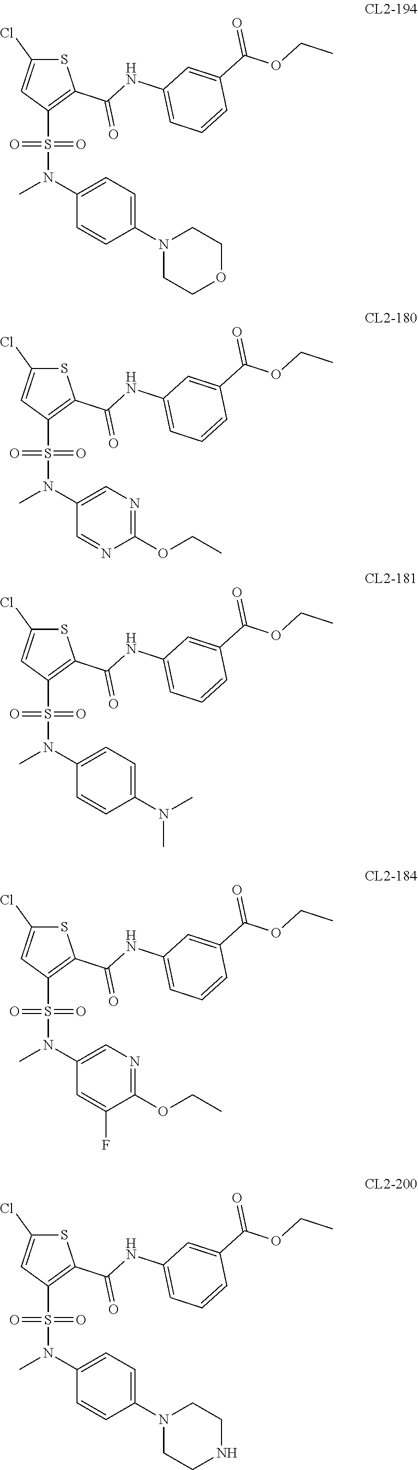

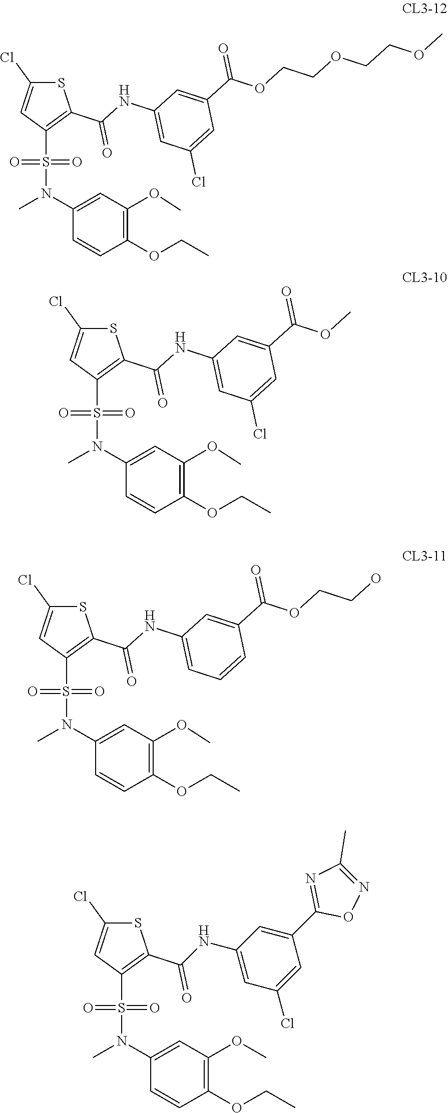 [SMILES: CCOc1ccc(N(C)S(=O)(=O)c2cc(Cl)sc2C(=O)Nc2cc(Cl)cc(C(=O)OCCOCCOC)c2)cc1OC ; CCOc1ccc(N(C)S(=O)(=O)c2cc(Cl)sc2C(=O)Nc2cc(Cl)cc(C(=O)OC)c2)cc1OC ; CCOc1ccc(N(C)S(=O)(=O)c2cc(Cl)sc2C(=O)Nc2cccc(C(=O)OCCO)c2)cc1OC ; CCOc1ccc(N(C)S(=O)(=O)c2cc(Cl)sc2C(=O)Nc2cc(Cl)cc(-c3nc(C)no3)c2)cc1OC]

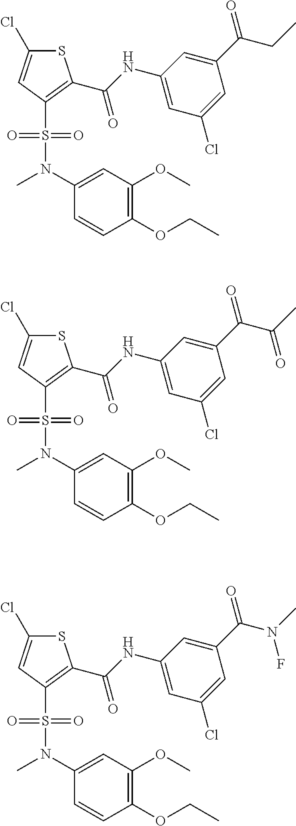 [SMILES: CCOc1ccc(N(C)S(=O)(=O)c2cc(Cl)sc2C(=O)Nc2cc(Cl)cc(C(=O)CC)c2)cc1OC ; CCOc1ccc(N(C)S(=O)(=O)c2cc(Cl)sc2C(=O)Nc2cc(Cl)cc(C(=O)C(C)=O)c2)cc1OC ; CCOc1ccc(N(C)S(=O)(=O)c2cc(Cl)sc2C(=O)Nc2cc(Cl)cc(C(=O)N(C)F)c2)cc1OC]

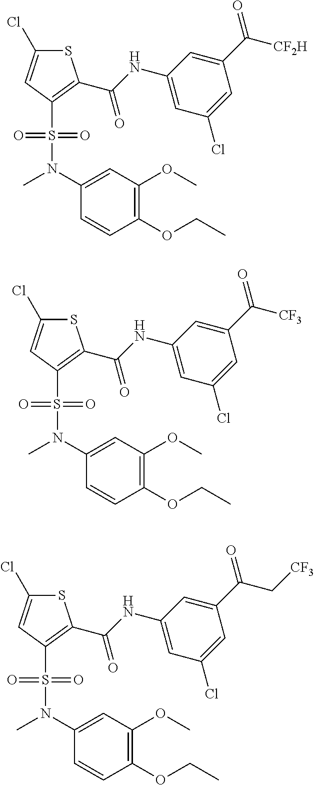

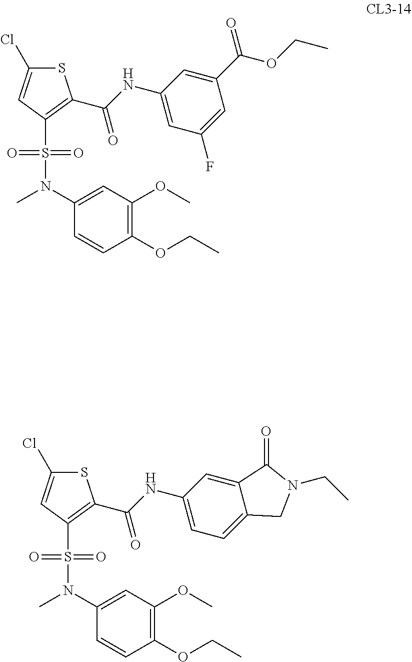 [SMILES: CCOC(=O)c1cc(F)cc(NC(=O)c2sc(Cl)cc2S(=O)(=O)N(C)c2ccc(OCC)c(OC)c2)c1 ; CCOc1ccc(N(C)S(=O)(=O)c2cc(Cl)sc2C(=O)Nc2ccc3c(c2)C(=O)N(CC)C3)cc1OC]

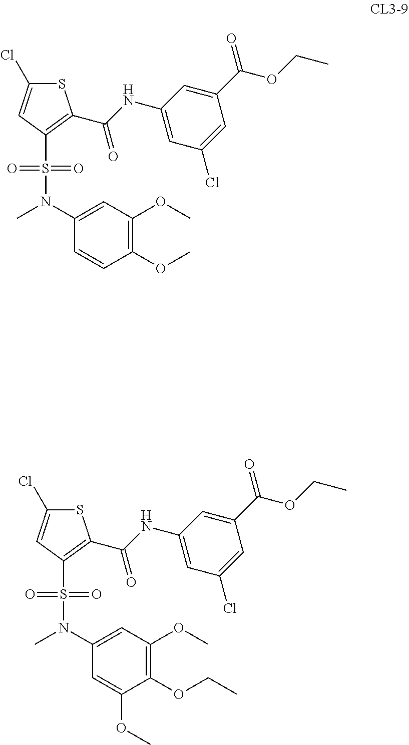 [SMILES: CCOC(=O)c1cc(Cl)cc(NC(=O)c2sc(Cl)cc2S(=O)(=O)N(C)c2ccc(OC)c(OC)c2)c1 ; CCOC(=O)c1cc(Cl)cc(NC(=O)c2sc(Cl)cc2S(=O)(=O)N(C)c2cc(OC)c(OCC)c(OC)c2)c1]

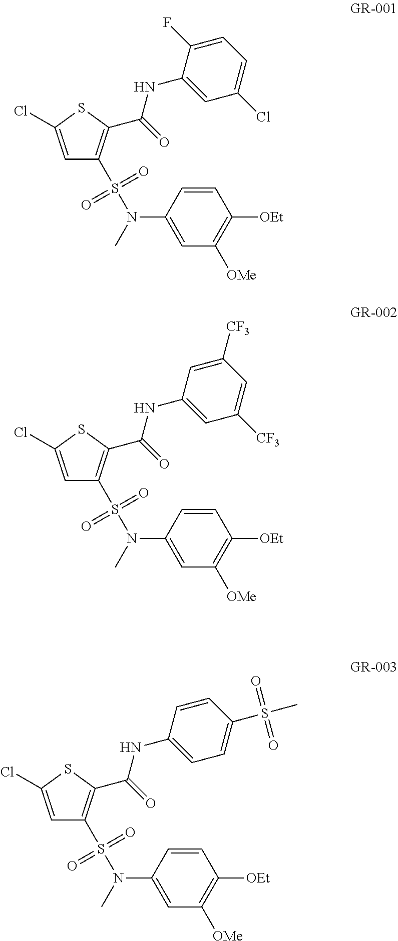 [SMILES: CCOc1ccc(N(C)S(=O)(=O)c2cc(Cl)sc2C(=O)Nc2cc(Cl)ccc2F)cc1OC ; CCOc1ccc(N(C)S(=O)(=O)c2cc(Cl)sc2C(=O)Nc2cc(C(F)(F)F)cc(C(F)(F)F)c2)cc1OC ; CCOc1ccc(N(C)S(=O)(=O)c2cc(Cl)sc2C(=O)Nc2ccc(S(C)(=O)=O)cc2)cc1OC]

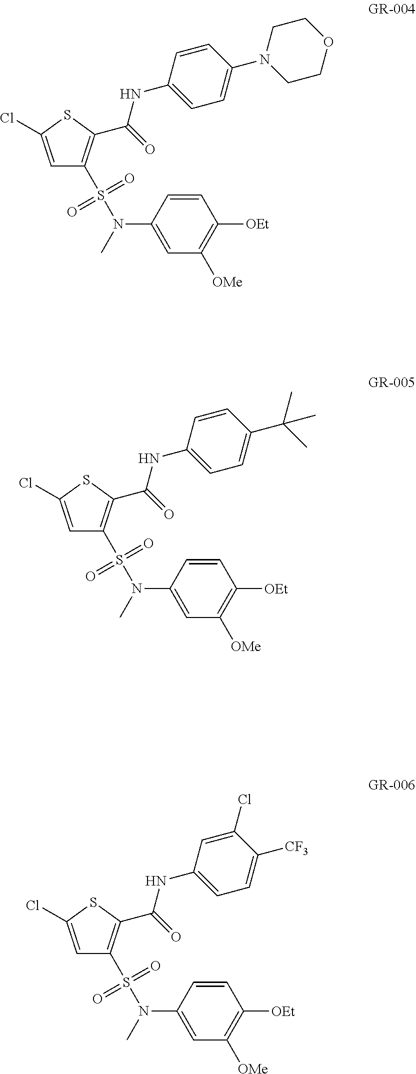 [SMILES: CCOc1ccc(N(C)S(=O)(=O)c2cc(Cl)sc2C(=O)Nc2ccc(N3CCOCC3)cc2)cc1OC ; CCOc1ccc(N(C)S(=O)(=O)c2cc(Cl)sc2C(=O)Nc2ccc(C(C)(C)C)cc2)cc1OC ; CCOc1ccc(N(C)S(=O)(=O)c2cc(Cl)sc2C(=O)Nc2ccc(C(F)(F)F)c(Cl)c2)cc1OC]

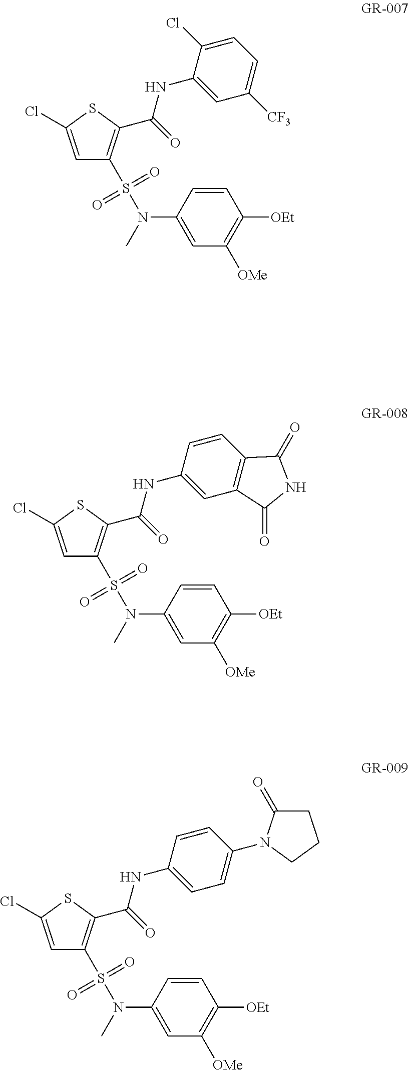 [SMILES: CCOc1ccc(N(C)S(=O)(=O)c2cc(Cl)sc2C(=O)Nc2cc(C(F)(F)F)ccc2Cl)cc1OC ; CCOc1ccc(N(C)S(=O)(=O)c2cc(Cl)sc2C(=O)Nc2ccc3c(c2)C(=O)NC3=O)cc1OC ; CCOc1ccc(N(C)S(=O)(=O)c2cc(Cl)sc2C(=O)Nc2ccc(N3CCCC3=O)cc2)cc1OC]

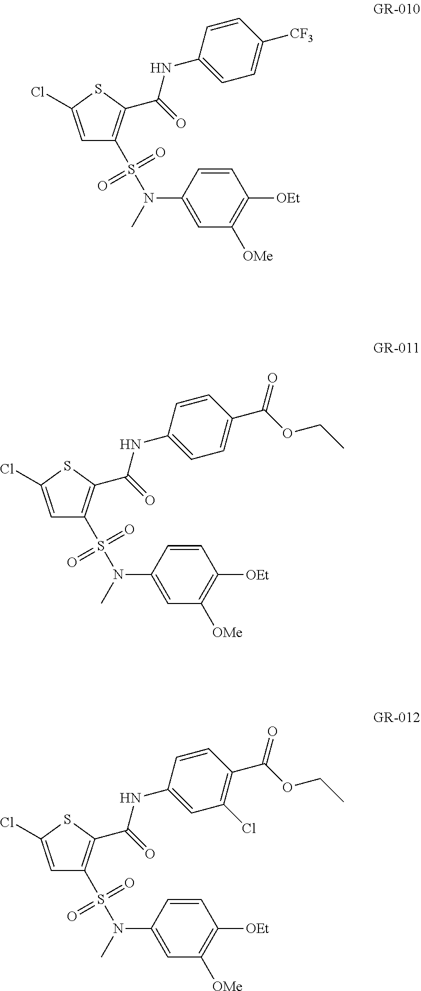 [SMILES: CCOc1ccc(N(C)S(=O)(=O)c2cc(Cl)sc2C(=O)Nc2ccc(C(F)(F)F)cc2)cc1OC ; CCOC(=O)c1ccc(NC(=O)c2sc(Cl)cc2S(=O)(=O)N(C)c2ccc(OCC)c(OC)c2)cc1 ; CCOC(=O)c1ccc(NC(=O)c2sc(Cl)cc2S(=O)(=O)N(C)c2ccc(OCC)c(OC)c2)cc1Cl]

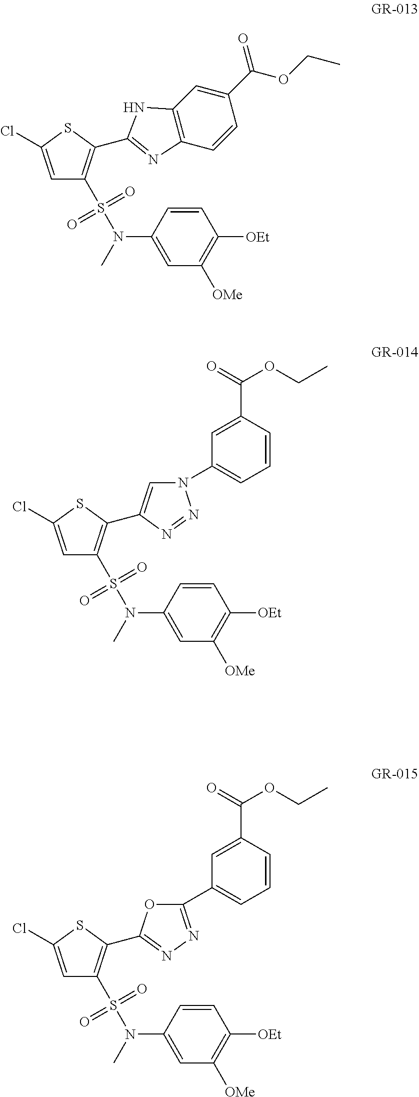 [SMILES: CCOC(=O)c1ccc2nc(-c3sc(Cl)cc3S(=O)(=O)N(C)c3ccc(OCC)c(OC)c3)[nH]c2c1 ; CCOC(=O)c1cccc(-n2cc(-c3sc(Cl)cc3S(=O)(=O)N(C)c3ccc(OCC)c(OC)c3)nn2)c1 ; CCOC(=O)c1cccc(-c2nnc(-c3sc(Cl)cc3S(=O)(=O)N(C)c3ccc(OCC)c(OC)c3)o2)c1]

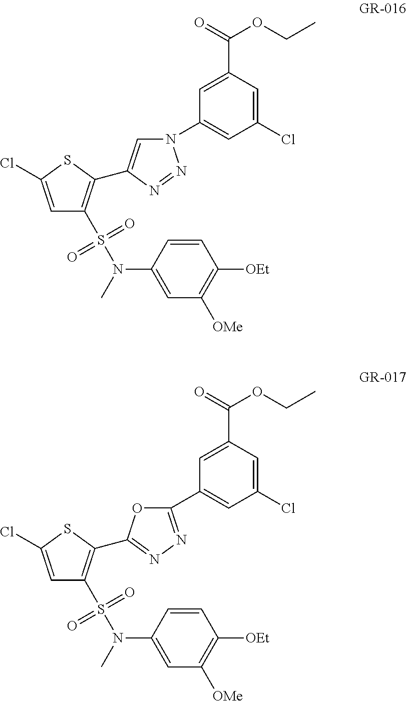 [SMILES: CCOC(=O)c1cc(Cl)cc(-n2cc(-c3sc(Cl)cc3S(=O)(=O)N(C)c3ccc(OCC)c(OC)c3)nn2)c1 ; CCOC(=O)c1cc(Cl)cc(-c2nnc(-c3sc(Cl)cc3S(=O)(=O)N(C)c3ccc(OCC)c(OC)c3)o2)c1]

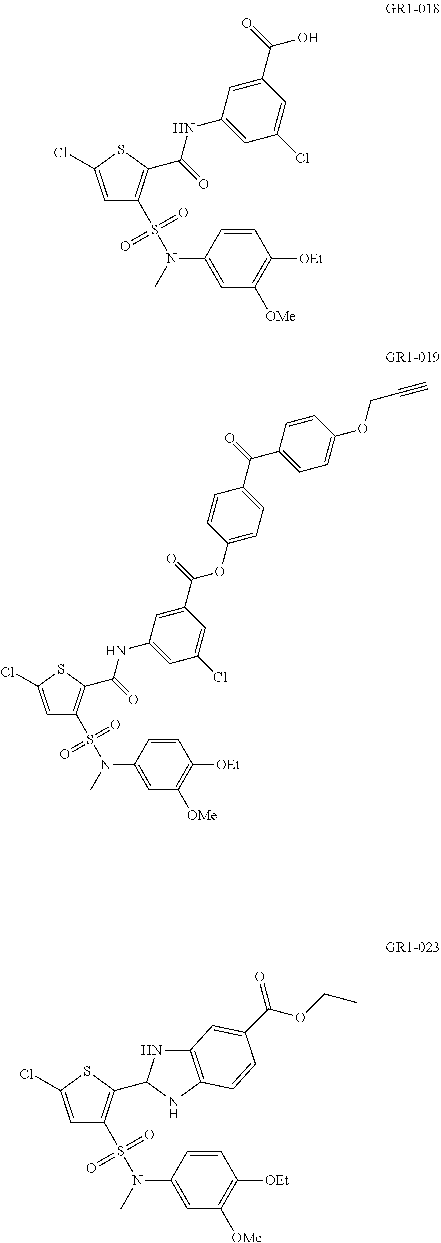 [SMILES: CCOc1ccc(N(C)S(=O)(=O)c2cc(Cl)sc2C(=O)Nc2cc(Cl)cc(C(=O)O)c2)cc1OC ; C#CCOc1ccc(C(=O)c2ccc(OC(=O)c3cc(Cl)cc(NC(=O)c4sc(Cl)cc4S(=O)(=O)N(C)c4ccc(OCC)c(OC)c4)c3)cc2)cc1 ; CCOC(=O)c1ccc2c(c1)NC(c1sc(Cl)cc1S(=O)(=O)N(C)c1ccc(OCC)c(OC)c1)N2]

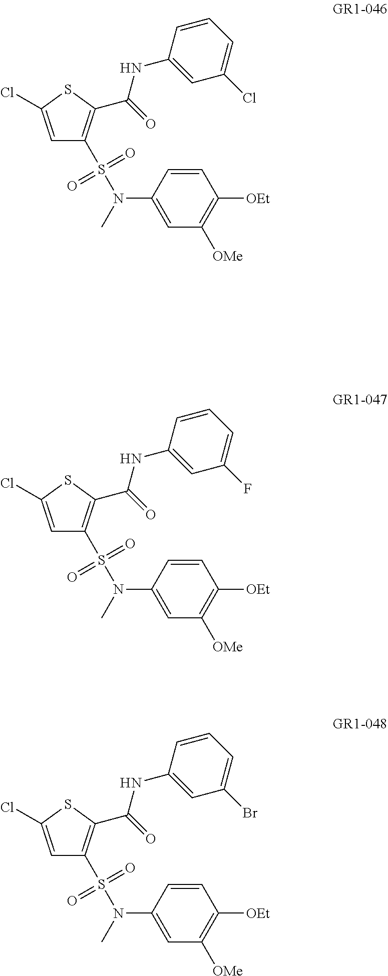 [SMILES: CCOc1ccc(N(C)S(=O)(=O)c2cc(Cl)sc2C(=O)Nc2cccc(Cl)c2)cc1OC ; CCOc1ccc(N(C)S(=O)(=O)c2cc(Cl)sc2C(=O)Nc2cccc(F)c2)cc1OC ; CCOc1ccc(N(C)S(=O)(=O)c2cc(Cl)sc2C(=O)Nc2cccc(Br)c2)cc1OC]

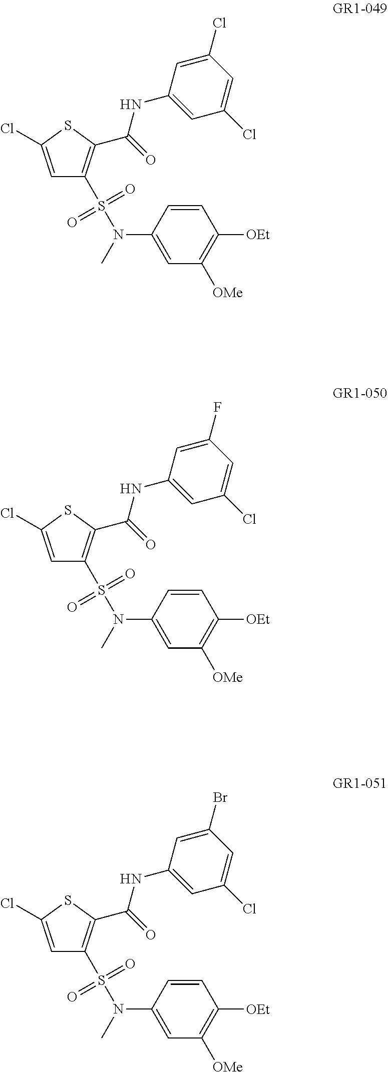 [SMILES: CCOc1ccc(N(C)S(=O)(=O)c2cc(Cl)sc2C(=O)Nc2cc(Cl)cc(Cl)c2)cc1OC ; CCOc1ccc(N(C)S(=O)(=O)c2cc(Cl)sc2C(=O)Nc2cc(F)cc(Cl)c2)cc1OC ; CCOc1ccc(N(C)S(=O)(=O)c2cc(Cl)sc2C(=O)Nc2cc(Cl)cc(Br)c2)cc1OC]

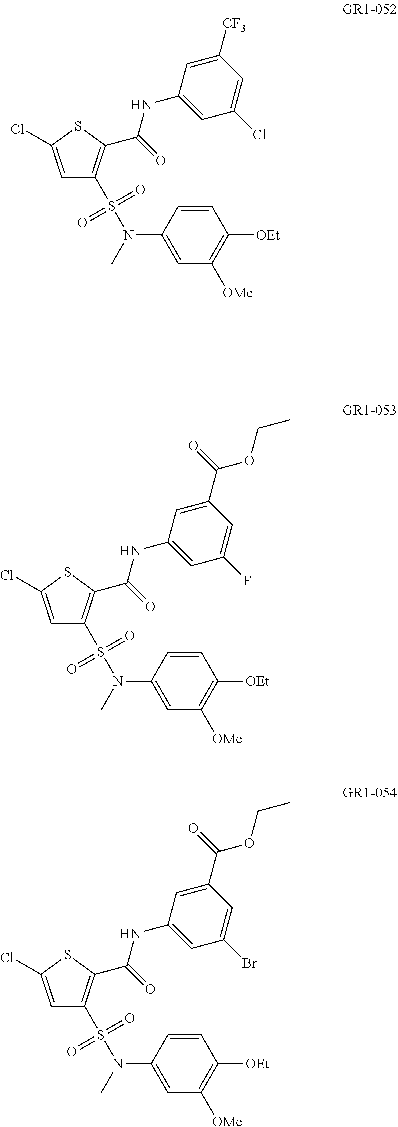 [SMILES: CCOc1ccc(N(C)S(=O)(=O)c2cc(Cl)sc2C(=O)Nc2cc(Cl)cc(C(F)(F)F)c2)cc1OC ; CCOC(=O)c1cc(F)cc(NC(=O)c2sc(Cl)cc2S(=O)(=O)N(C)c2ccc(OCC)c(OC)c2)c1 ; CCOC(=O)c1cc(Br)cc(NC(=O)c2sc(Cl)cc2S(=O)(=O)N(C)c2ccc(OCC)c(OC)c2)c1]

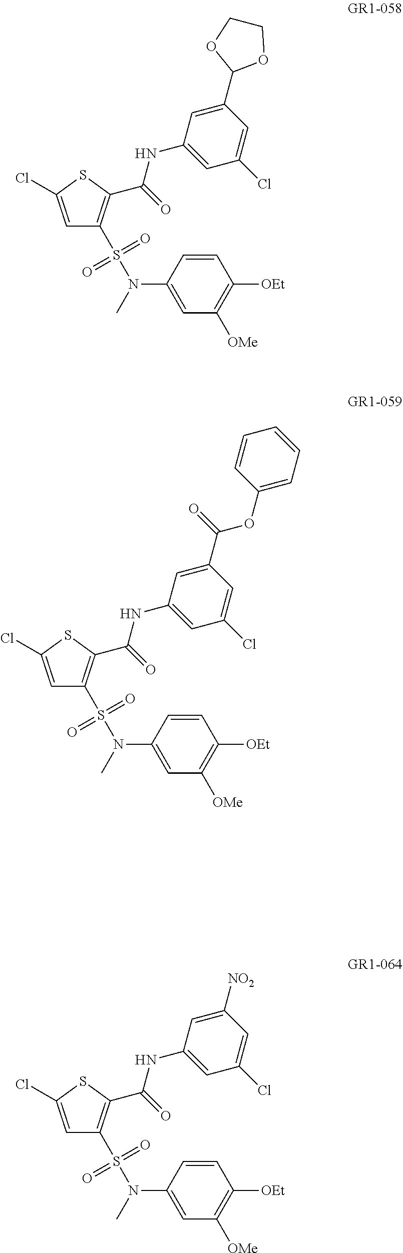 [SMILES: CCOc1ccc(N(C)S(=O)(=O)c2cc(Cl)sc2C(=O)Nc2cc(Cl)cc(C3OCCO3)c2)cc1OC ; CCOc1ccc(N(C)S(=O)(=O)c2cc(Cl)sc2C(=O)Nc2cc(Cl)cc(C(=O)Oc3ccccc3)c2)cc1OC ; CCOc1ccc(N(C)S(=O)(=O)c2cc(Cl)sc2C(=O)Nc2cc(Cl)cc([N+](=O)[O-])c2)cc1OC]

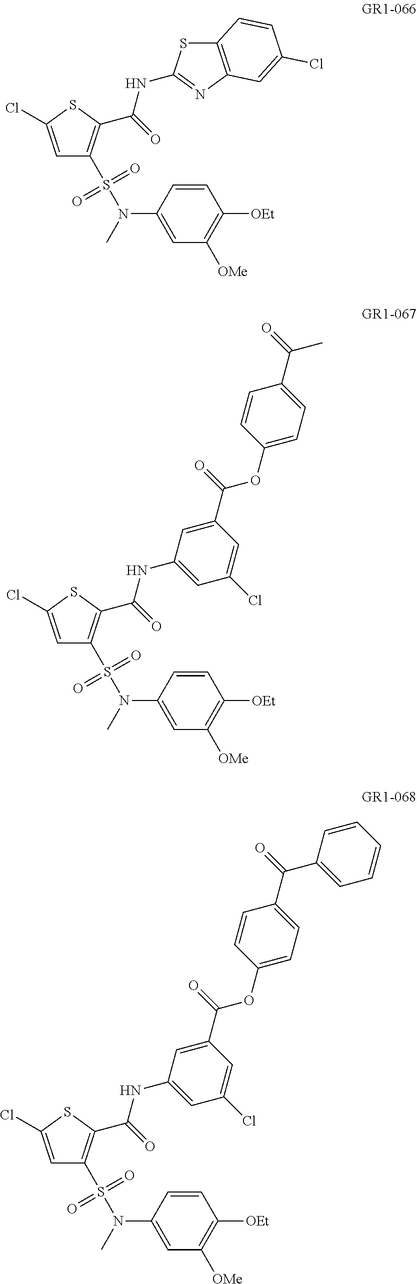 [SMILES: CCOc1ccc(N(C)S(=O)(=O)c2cc(Cl)sc2C(=O)Nc2nc3cc(Cl)ccc3s2)cc1OC ; CCOc1ccc(N(C)S(=O)(=O)c2cc(Cl)sc2C(=O)Nc2cc(Cl)cc(C(=O)Oc3ccc(C(C)=O)cc3)c2)cc1OC ; CCOc1ccc(N(C)S(=O)(=O)c2cc(Cl)sc2C(=O)Nc2cc(Cl)cc(C(=O)Oc3ccc(C(=O)c4ccccc4)cc3)c2)cc1OC]

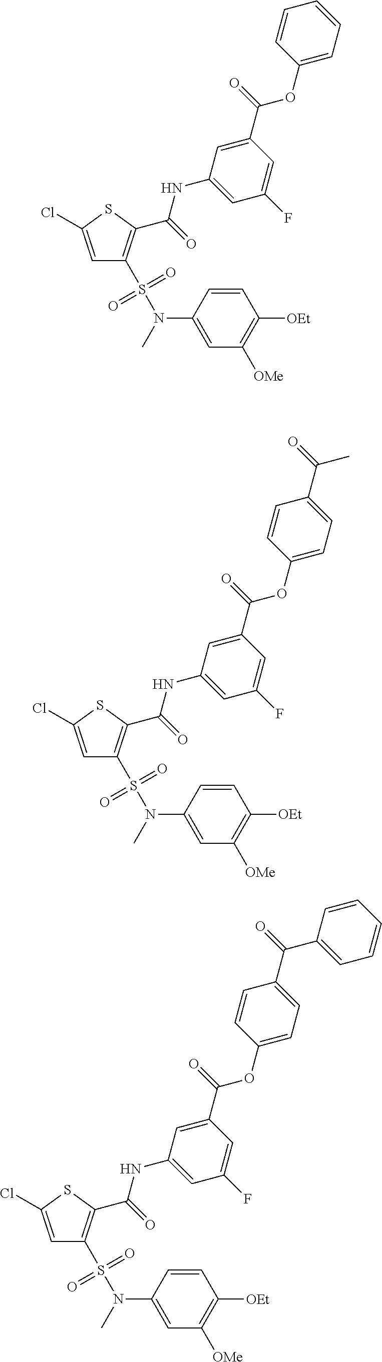 [SMILES: CCOc1ccc(N(C)S(=O)(=O)c2cc(Cl)sc2C(=O)Nc2cc(F)cc(C(=O)Oc3ccccc3)c2)cc1OC ; CCOc1ccc(N(C)S(=O)(=O)c2cc(Cl)sc2C(=O)Nc2cc(F)cc(C(=O)Oc3ccc(C(C)=O)cc3)c2)cc1OC ; CCOc1ccc(N(C)S(=O)(=O)c2cc(Cl)sc2C(=O)Nc2cc(F)cc(C(=O)Oc3ccc(C(=O)c4ccccc4)cc3)c2)cc1OC]

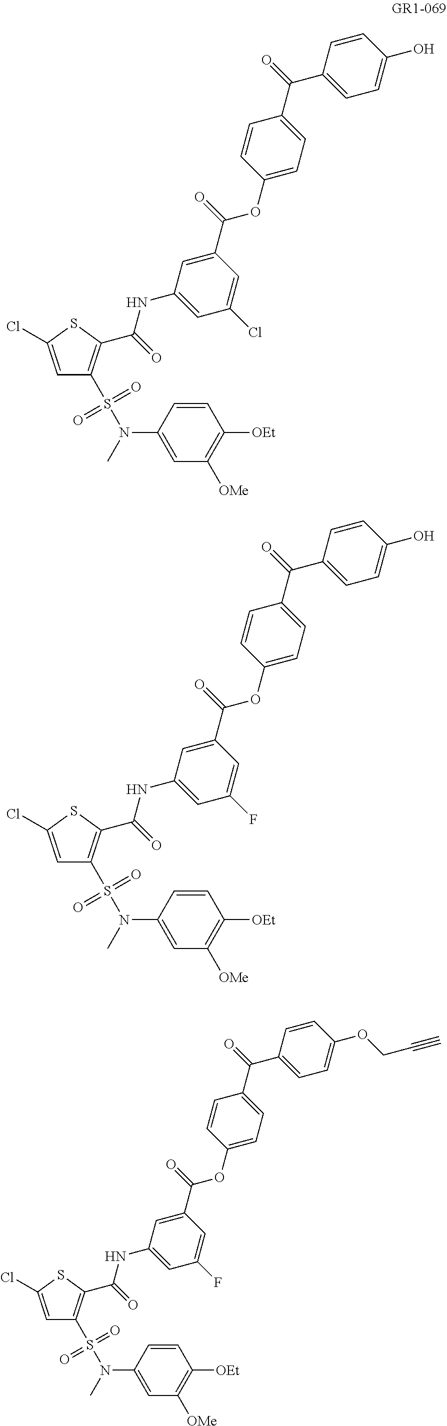 [SMILES: CCOc1ccc(N(C)S(=O)(=O)c2cc(Cl)sc2C(=O)Nc2cc(Cl)cc(C(=O)Oc3ccc(C(=O)c4ccc(O)cc4)cc3)c2)cc1OC ; CCOc1ccc(N(C)S(=O)(=O)c2cc(Cl)sc2C(=O)Nc2cc(F)cc(C(=O)Oc3ccc(C(=O)c4ccc(O)cc4)cc3)c2)cc1OC ; C#CCOc1ccc(C(=O)c2ccc(OC(=O)c3cc(F)cc(NC(=O)c4sc(Cl)cc4S(=O)(=O)N(C)c4ccc(OCC)c(OC)c4)c3)cc2)cc1]

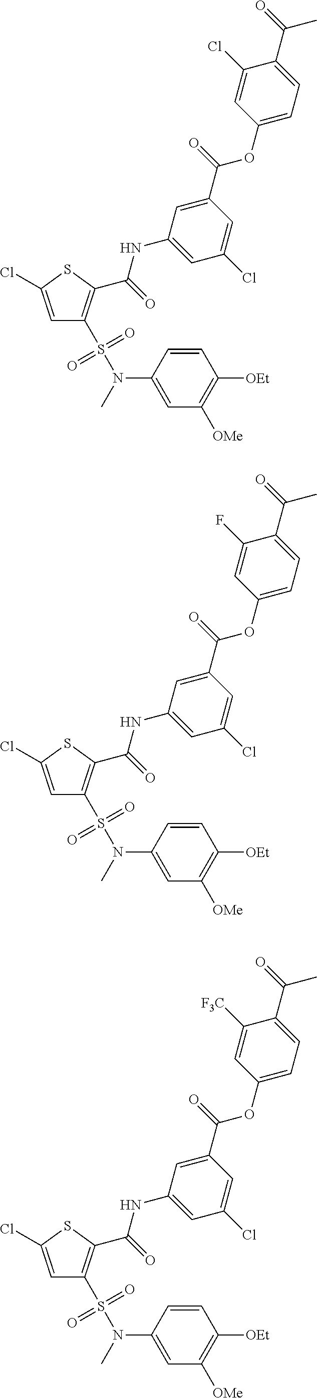 [SMILES: CCOc1ccc(N(C)S(=O)(=O)c2cc(Cl)sc2C(=O)Nc2cc(Cl)cc(C(=O)Oc3ccc(C(C)=O)c(Cl)c3)c2)cc1OC ; CCOc1ccc(N(C)S(=O)(=O)c2cc(Cl)sc2C(=O)Nc2cc(Cl)cc(C(=O)Oc3ccc(C(C)=O)c(F)c3)c2)cc1OC ; CCOc1ccc(N(C)S(=O)(=O)c2cc(Cl)sc2C(=O)Nc2cc(Cl)cc(C(=O)Oc3ccc(C(C)=O)c(C(F)(F)F)c3)c2)cc1OC]

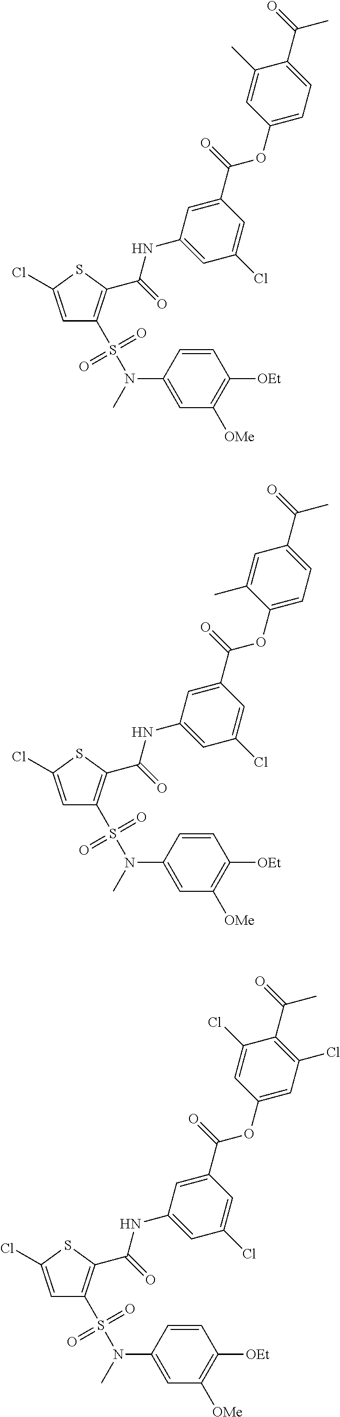 [SMILES: CCOc1ccc(N(C)S(=O)(=O)c2cc(Cl)sc2C(=O)Nc2cc(Cl)cc(C(=O)Oc3ccc(C(C)=O)c(C)c3)c2)cc1OC ; CCOc1ccc(N(C)S(=O)(=O)c2cc(Cl)sc2C(=O)Nc2cc(Cl)cc(C(=O)Oc3ccc(C(C)=O)cc3C)c2)cc1OC ; CCOc1ccc(N(C)S(=O)(=O)c2cc(Cl)sc2C(=O)Nc2cc(Cl)cc(C(=O)Oc3cc(Cl)c(C(C)=O)c(Cl)c3)c2)cc1OC]

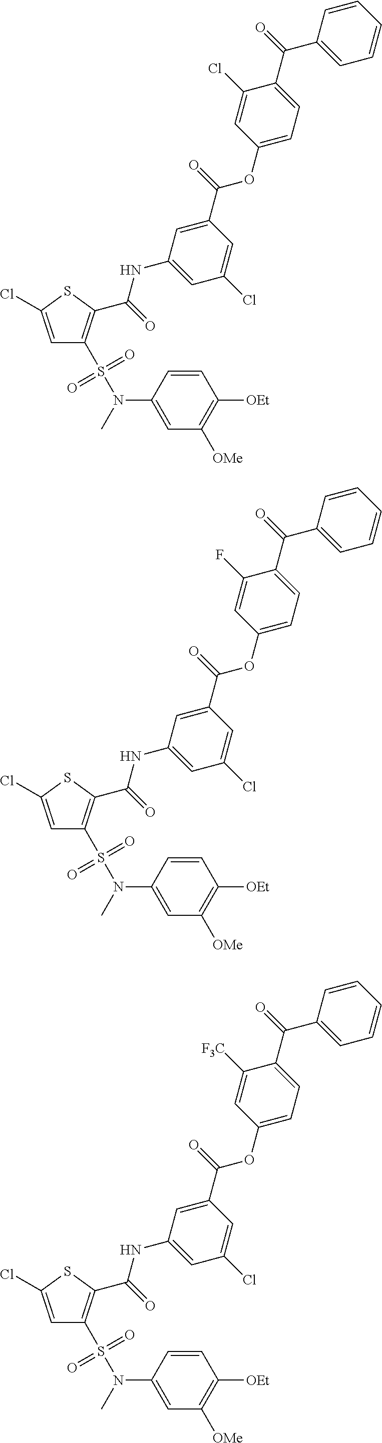 [SMILES: CCOc1ccc(N(C)S(=O)(=O)c2cc(Cl)sc2C(=O)Nc2cc(Cl)cc(C(=O)Oc3ccc(C(=O)c4ccccc4)c(Cl)c3)c2)cc1OC ; CCOc1ccc(N(C)S(=O)(=O)c2cc(Cl)sc2C(=O)Nc2cc(Cl)cc(C(=O)Oc3ccc(C(=O)c4ccccc4)c(F)c3)c2)cc1OC ; CCOc1ccc(N(C)S(=O)(=O)c2cc(Cl)sc2C(=O)Nc2cc(Cl)cc(C(=O)Oc3ccc(C(=O)c4ccccc4)c(C(F)(F)F)c3)c2)cc1OC]

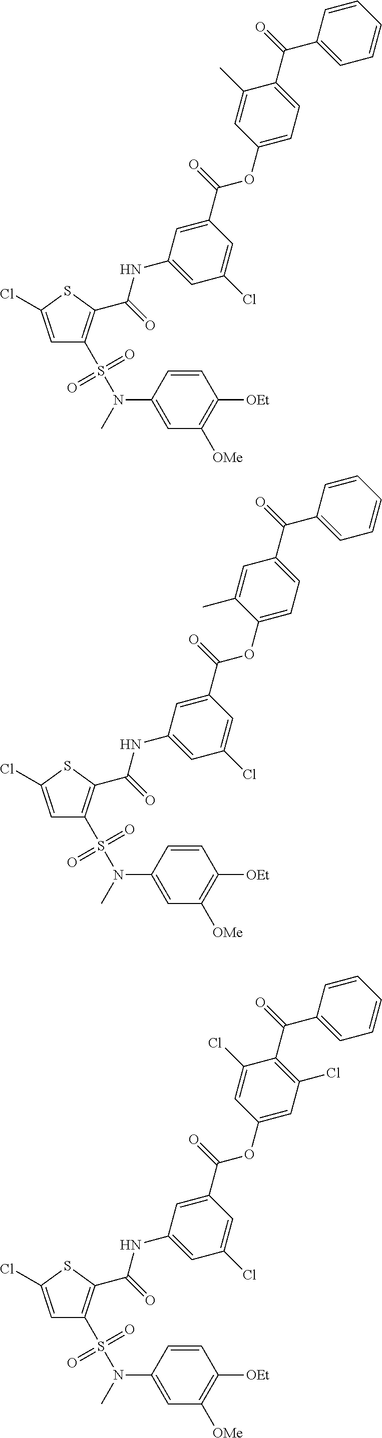 [SMILES: CCOc1ccc(N(C)S(=O)(=O)c2cc(Cl)sc2C(=O)Nc2cc(Cl)cc(C(=O)Oc3ccc(C(=O)c4ccccc4)c(C)c3)c2)cc1OC ; CCOc1ccc(N(C)S(=O)(=O)c2cc(Cl)sc2C(=O)Nc2cc(Cl)cc(C(=O)Oc3ccc(C(=O)c4ccccc4)cc3C)c2)cc1OC ; CCOc1ccc(N(C)S(=O)(=O)c2cc(Cl)sc2C(=O)Nc2cc(Cl)cc(C(=O)Oc3cc(Cl)c(C(=O)c4ccccc4)c(Cl)c3)c2)cc1OC]

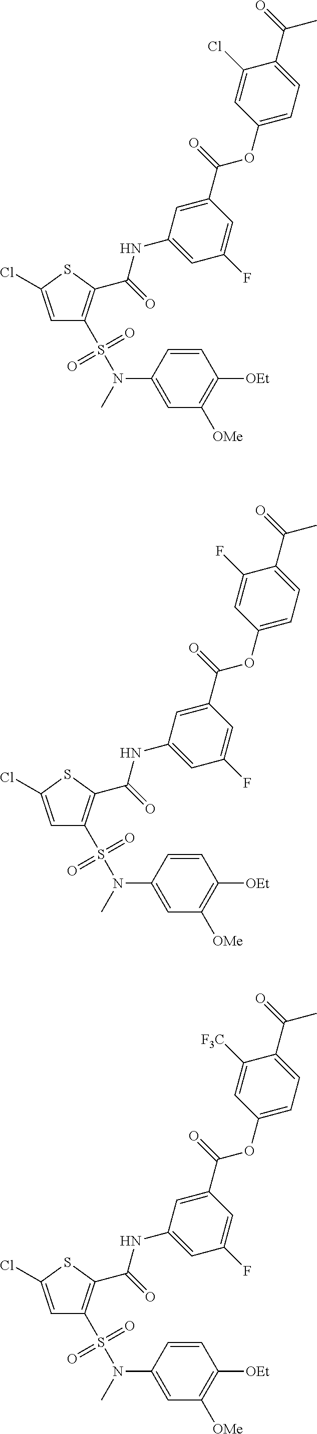 [SMILES: CCOc1ccc(N(C)S(=O)(=O)c2cc(Cl)sc2C(=O)Nc2cc(F)cc(C(=O)Oc3ccc(C(C)=O)c(Cl)c3)c2)cc1OC ; CCOc1ccc(N(C)S(=O)(=O)c2cc(Cl)sc2C(=O)Nc2cc(F)cc(C(=O)Oc3ccc(C(C)=O)c(F)c3)c2)cc1OC ; CCOc1ccc(N(C)S(=O)(=O)c2cc(Cl)sc2C(=O)Nc2cc(F)cc(C(=O)Oc3ccc(C(C)=O)c(C(F)(F)F)c3)c2)cc1OC]

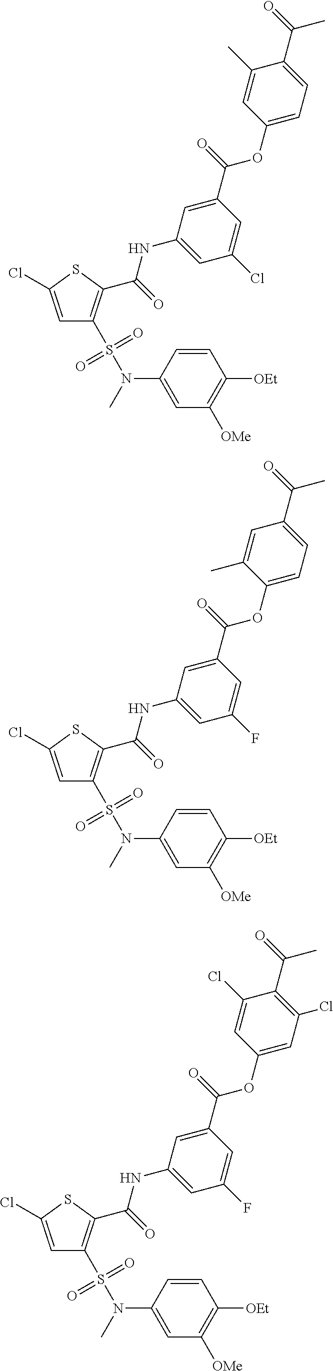 [SMILES: CCOc1ccc(N(C)S(=O)(=O)c2cc(Cl)sc2C(=O)Nc2cc(Cl)cc(C(=O)Oc3ccc(C(C)=O)c(C)c3)c2)cc1OC ; CCOc1ccc(N(C)S(=O)(=O)c2cc(Cl)sc2C(=O)Nc2cc(F)cc(C(=O)Oc3ccc(C(C)=O)cc3C)c2)cc1OC ; CCOc1ccc(N(C)S(=O)(=O)c2cc(Cl)sc2C(=O)Nc2cc(F)cc(C(=O)Oc3cc(Cl)c(C(C)=O)c(Cl)c3)c2)cc1OC]

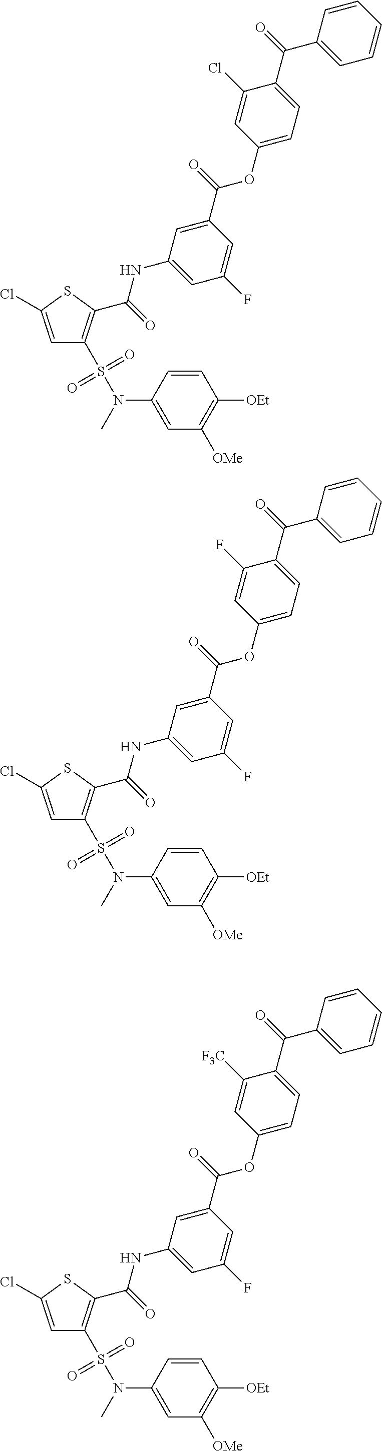 [SMILES: CCOc1ccc(N(C)S(=O)(=O)c2cc(Cl)sc2C(=O)Nc2cc(F)cc(C(=O)Oc3ccc(C(=O)c4ccccc4)c(Cl)c3)c2)cc1OC ; CCOc1ccc(N(C)S(=O)(=O)c2cc(Cl)sc2C(=O)Nc2cc(F)cc(C(=O)Oc3ccc(C(=O)c4ccccc4)c(F)c3)c2)cc1OC ; CCOc1ccc(N(C)S(=O)(=O)c2cc(Cl)sc2C(=O)Nc2cc(F)cc(C(=O)Oc3ccc(C(=O)c4ccccc4)c(C(F)(F)F)c3)c2)cc1OC]

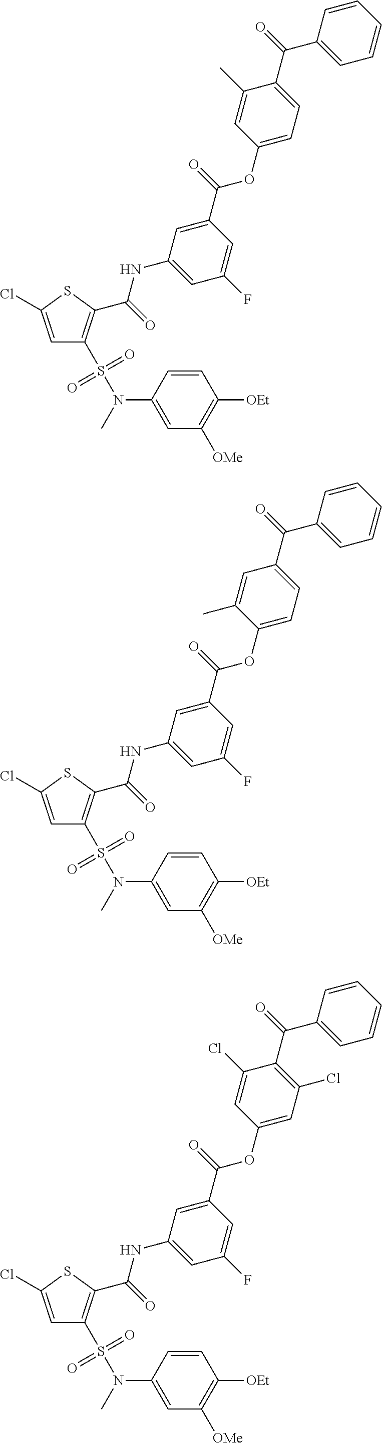 [SMILES: CCOc1ccc(N(C)S(=O)(=O)c2cc(Cl)sc2C(=O)Nc2cc(F)cc(C(=O)Oc3ccc(C(=O)c4ccccc4)c(C)c3)c2)cc1OC ; CCOc1ccc(N(C)S(=O)(=O)c2cc(Cl)sc2C(=O)Nc2cc(F)cc(C(=O)Oc3ccc(C(=O)c4ccccc4)cc3C)c2)cc1OC ; CCOc1ccc(N(C)S(=O)(=O)c2cc(Cl)sc2C(=O)Nc2cc(F)cc(C(=O)Oc3cc(Cl)c(C(=O)c4ccccc4)c(Cl)c3)c2)cc1OC]

In one aspect, the compound of this invention is CL2-57 or CL3-3, or an analog or salt thereof. In another aspect, the compound of this invention does not include F420. In a further aspect, the compound of this invention does not include CL1-48, CL1-49, CL1-63, CL1-64, CL1-84, CL1-89, CL1-96, CL1-116, CL1-121, CL1-122, CL1-123, CL2-4, CL2-19, CL2-22, CL2-23, CL2-37, CL2-44, CL2-66, CL2-70, CL2-129, CL2-144, CL2-145, CL2-161, CL2-163, CL2-175, CL2-178, CL2-180, CL2-194, or CL3-1.

This invention also encompasses a composition including one or more compounds of Formula (I), Formula (II), Formula (III), Formula (IV), Formula (V), Formula (VI), Formula (VII), or Formula (VIII), Formula (IX), Formula (X), or Formula (XI), or a pharmaceutically acceptable salt thereof, in admixture with one or more pharmaceutically acceptable diluents, preservatives, solubilizers, emulsifiers, adjuvants, excipients, or carriers. Ideally, a pharmaceutical composition includes a therapeutically effective amount of one or more compounds of Formula (I), Formula (II), Formula (III), Formula (IV), Formula (V), Formula (VI), Formula (VII), or Formula (VIII), Formula (IX), Formula (X), or Formula (XI), or a pharmaceutically acceptable salt or prodrug thereof as described herein. The pharmaceutical composition can be used, for example, in treating one or more diseases or conditions, where benefit is attained by agonizing LXRβ and/or increasing ABCA1 mRNA and protein levels.

The term “pharmaceutically acceptable vehicle” refers to a diluent, adjuvant, excipient or carrier with which a compound of the disclosure is administered. The term “effective amount,” “pharmaceutically effective amount” or “therapeutically effective amount” refers to a nontoxic but sufficient amount of the agent to provide the desired biological result. That result can be reduction and/or alleviation of the signs, symptoms, or causes of a disease, or any other desired alteration of a biological system. An appropriate “effective” amount in any individual case can be determined by one of ordinary skill in the art using routine experimentation.

Pharmaceutically acceptable carriers or vehicles for therapeutic use are well known in the pharmaceutical art, and are described, for example, in Remington's Pharmaceutical Sciences, 18th Edition (Easton, Pennsylvania: Mack Publishing Company, 1990). For example, sterile saline and phosphate-buffered saline at physiological pH can be used.

Preservatives, stabilizers, dyes and even flavoring agents can be provided in the pharmaceutical composition. For example, sodium benzoate, sorbic acid and esters of p-hydroxybenzoic acid can be added as preservatives. Id. at 1449. In addition, antioxidants and suspending agents can be used. Id.

Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semisolid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, can include other pharmaceutical agents, adjuvants, diluents, buffers, and the like.

In general, the compositions of the disclosure will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

Thus, the compositions of the disclosure can be administered as pharmaceutical formulations including those suitable for oral (including buccal and sub-lingual), rectal, nasal, topical, pulmonary, vaginal or parenteral (including intramuscular, intra-arterial, intrathecal, subcutaneous and intravenous) administration or in a form suitable for administration by inhalation or insufflation. The preferred manner of administration is intravenous or oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another aspect is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacterium retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Preferred formulations for topical drug delivery are ointments and creams. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Formulations for buccal administration include tablets, lozenges, gels and the like. Alternatively, buccal administration can be affected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface.

In such a structure, the drug composition is typically contained in a layer, or “reservoir,” underlying an upper backing layer. The laminated device can contain a single reservoir, or it can contain multiple reservoirs. In one aspect, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound will generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve. Alternatively, the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of, e.g., gelatin or blister packs from which the powder can be administered by means of an inhaler.

A pharmaceutically or therapeutically effective amount of the composition will be delivered to the subject. The precise effective amount will vary from subject to subject and will depend upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the disclosure, generally a therapeutic amount will be in the range of about 0.01 mg/kg to about 250 mg/kg body weight, more preferably about 0.1 mg/kg to about 10 mg/kg, in at least one dose. In larger mammals the indicated daily dosage can be from about 1 mg to 300 mg, one or more times per day, more preferably in the range of about 10 mg to 200 mg. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Compounds of the present disclosure have been shown to induce cellular ABCA1 expression without lipogenesis; agonize LXRP; and/or ameliorate one or more signs and symptoms associated with T2D and/or dementia. Thus, the compounds of this invention find use in the preparation of a medicament for the treatment of one or more diseases or conditions, and in methods for treating such diseases or conditions, in particular diseases or conditions in which benefit may be derived from inducing cellular ABCA1 expression, agonizing LXR3, decreasing or reducing inflammation in a subject, e.g., as evidenced by reduced TNFα and NOS2 expression; enhancing glucose tolerance and/or insulin sensitivity; reducing weight gain and adiposity associated with a high-fat diet; reducing plasma and/or liver triglyceride levels; and/or attenuating pro-inflammatory cytokines, enzymes and metabolites compared to a subject not receiving treatment with said compound.

Thus, in one aspect, this invention provides a method for increasing the expression of ABCA1 in a cell or subject by administering to the cell or subject an effective amount of compound of Formula (I), Formula (II), Formula (III), Formula (IV), Formula (V), Formula (VI), Formula (VII), or Formula (VIII), Formula (IX), Formula (X), Formula (XI) or a pharmaceutically acceptable salt thereof. Desirably, the expression of ABCA1 is increase by at least 1.5-fold or 2.0-fold compared to a cell or subject not treated with the compound. Increases in ABCA1 expression can be assessed by measuring mRNA or protein levels of ABCA1 in accordance with conventional methods.

Compounds of the present disclosure have been shown to agonize LXRβ and antagonize PPAR/RXR without lipogenesis thereby providing a treatment for diseases or conditions in a subject for which enhanced LXRβ activity and reduced activity of PPAR/RXR provides a benefit. Thus, in another aspect, this invention provides a method for agonizing LXRβ by administering to the cell or subject an effective amount of a compound of Formula (I), Formula (II), Formula (III), Formula (IV), Formula (V), Formula (VI), Formula (VII), or Formula (VIII), Formula (IX), Formula (X), Formula (XI) or a pharmaceutically acceptable salt thereof. Desirably, the agonist is selective for LXRβ over LXRα. Ideally, the agonist exhibits at least 2.5-fold selectively for LXRβ over LXRα.

In further aspects, this invention provides methods of treating or preventing T2D or dementia by administering to a subject, typically a human, in need of such treatment, an effective amount of one or more compounds of Formula (I), Formula (II), Formula (III), Formula (IV), Formula (V), Formula (VI), Formula (VII), or Formula (VIII), Formula (IX), Formula (X), Formula (XI) or a pharmaceutically acceptable salt thereof.

“Treating” or “treatment” as used herein covers the treatment of a disease or disorder described herein, in a subject, preferably a human, and includes inhibiting a disease or disorder, i.e., arresting its development; relieving a disease or disorder, i.e., causing regression of the disorder; slowing progression of the disorder; and/or inhibiting, relieving, or slowing progression of one or more symptoms of the disease or disorder. In some aspects, a compound of this inventions measurably (i.e., at a statistically significant level) decreases or reduces inflammation in a subject, e.g., as evidenced by reduced TNFα and NOS2 expression; enhances glucose tolerance and/or insulin sensitivity; reduces weight gain and adiposity associated with a high-fat diet; reduces plasma and/or liver triglyceride levels; and attenuates pro-inflammatory cytokines, enzymes and metabolites compared to a subject not receiving treatment with said compound.

“Subject” refers to a warm-blooded animal such as a mammal, preferably a human, or a human child, which is afflicted with, or has the potential to be afflicted with one or more diseases and disorders described herein. Subjects benefiting from any one of the above referenced treatments include, but are not limited to, subjects at risk of having, having or predisposed to have sporadic or late-onset Alzheimer's disease and related dementia, obesity, type 2 diabetes (T2D), or cardiovascular and cerebrovascular disease (CVD).

The following non-limiting examples are provided to further illustrate the present invention.

### Example 1: Synthesis of F420

Ethyl 3-(3-(N-(4-ethoxyphenyl)-N-methylsulfamoyl)thiophene-2-carboxamido) benzoate (F420). F420 was synthesized as described in Aissa et al. (2021) ACS Pharmacol. Transl. Sci. 4(1):143-154 (Scheme 1).

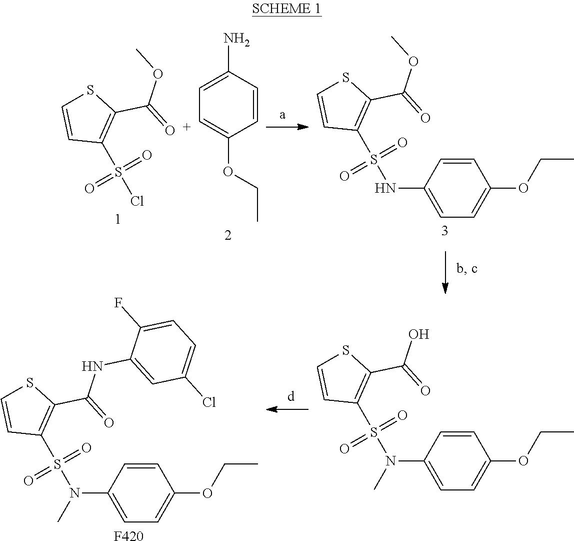

Conditions: (a) Et₃N, THF, 24 h, rt. (b) NaH/CH₃I, DMF, 24 h, 0° C. to rt. (c) NaOH/H₂O, 24 h, rt. (d) C₀₂C₁₂/ethyl 3-amino benzoate, DCM, 24 h, 40° C. to rt. Reagents in first step, methyl 3-(chlorosulfonyl)thiophene-2-carboxylate (1) and 4-ethoxyaniline (2), yielded methyl 3-(N-(4-ethoxyphenyl)sulfamoyl)thiophene-2-carboxylate (3). ¹H NMR (400 MHz, CDCl₃) δ=1.38 (t, 3H, J=7.0 Hz), 3.97 (q, 4H, J=7.0 Hz), 4.04 (s, 3H), 6.76 (d, 2H, J=8.8 Hz), 7.04 (d, 2H, J=8.8 Hz), 7.40 (d, 1H, J=5.0 Hz), 7.44 (d, 1H, 5.3 Hz), 8.12 (s, 1H). Following sulfonamide methylation: ¹H NMR (400 MHz, CDCl₃) δ=1.41 (t, 3H, J=7.4 Hz), 3.41 (s, 3H), 3.88 (s, 3H), 4.02 (q, 2H, J=6.9 Hz), 6.81 (d, 2H, J=8.9 Hz), 7.11 (d, 2H, J=8.7 Hz), 7.15 (d, 1H, J=5.2 Hz), 7.38 (d, 1H, J=5.2 Hz). After ester hydrolysis: ¹H NMR (400 MHz, CDCl₃) δ=1.43 (t, 3H, J=7.0 Hz), 3.28 (s, 3H), 4.04 (q, 2H, J=7.2 Hz), 6.86 (dd, 2H, J₁=8.8 Hz, J₂=2.5 Hz), 6.97 (dd, 2H, J₁=8.7 Hz, J₂=2.4 Hz), 7.30 (d, 1H, J=5.3 Hz), 7.67 (d, 1H, J=5.3 Hz). Final step used ethyl 3-aminobenzoate. ¹H NMR (400 MHz, CDCl₃) δ=1.25 (t, 3H, J=7.0 Hz), 1.44 (t, 3H, J=7.1 Hz), 3.20 (s, 3H), 3.62 (q, 2H, J=7.0 Hz), 4.42 (q, 2H, J=7.2 Hz), 6.57 (dd, 2H, J₁=8.9 Hz, J₂=2.5 Hz), 7.01 (dd, 2H, J₁=9.0 Hz, J₂=2.5 Hz), 7.34 (t, 1H, J=7.9 Hz), 7.50 (d, 1H, J=5.3 Hz), 7.61 (d, 1H, J=5.3 Hz), 7.63 (ddd, 1H, J₁=8.0 Hz, J₂=2.0 Hz, J₃=1.7 Hz), 7.80 (ddd, 1H, J₁=7.9 Hz, J₂=2.4 Hz, J₃=1.3 Hz), 8.03 (t, 1H, J=1.8 Hz), 10.08 (s, 1H). ¹³C NMR (100 MHz, CDCl₃) δ=14.4, 14.5, 38.6, 61.1, 63.4, 115.0, 120.6, 124.0, 125.4, 128.2, 128.5, 129.3, 131.0, 131.8, 132.06, 132.13, 137.7, 144.1, 157.4, 158.9, 166.1. LC-MS (m/z): calc. for C₂₃H₂₅N₂OS₂ [M+H⁺]: 489.11, found: 489.0. HPLC purity: >99%.

### Example 2: Synthesis and Characterization of Compounds Active in ABCA1-Luc Assay

Synthesis of ethyl 3-(5-chloro-3-(N-(3,4-diethoxyphenyl)-N-methylsulfamoyl)thiophene-2-carboxamido) benzoate (CL2-57). A three-step synthetic approach was used in the preparation of CL2-57 (Scheme 2).

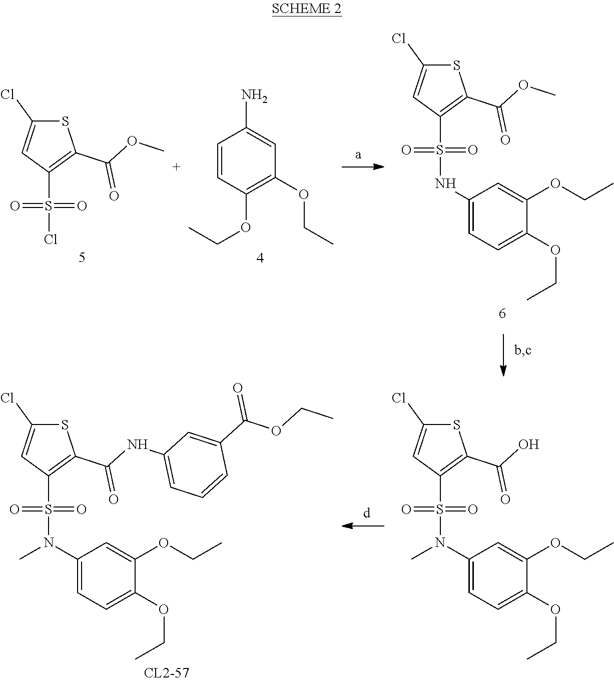 [SMILES: COC(=O)c1sc(Cl)cc1S(=O)(=O)Cl ; CCOc1ccc(N)cc1OCC ; CCOc1ccc(NS(=O)(=O)c2cc(Cl)sc2C(=O)OC)cc1OCC ; CCOC(=O)c1cccc(NC(=O)c2sc(Cl)cc2S(=O)(=O)N(C)c2ccc(OCC)c(OCC)c2)c1 ; CCOc1ccc(N(C)S(=O)(=O)c2cc(Cl)sc2C(=O)O)cc1OCC]

Conditions: (a) Et₃N, THF, rt, 24 h. (b) NaH, CH₃I, DMF, Ar₂, 0° C. to rt, 24 hr. (c) NaOH, H₂O, rt, 24 hr. (d) oxalyl chloride, cat. DMF, ethyl 3-aminobenzoate, Et₃N, DCM, 40° C. to rt, 24 h. 58% yield overall across three steps.

Reagents in the first step, 3,4-diethoxyaniline (4) and methyl 5-chloro-3-(chlorosulfonyl)thiophene-2-carboxylate (5), yielded methyl 5-chloro-3-(N-(3,4-diethoxyphenyl)sulfamoyl)thiophene-2-carboxylate (6). ¹H NMR (400 MHz, CDCl₃) δ=1.43 (t, 3H, J=7.0 Hz), 1.44 (t, 3H, J=7.0 Hz), 4.01 (s, 3H), 4.03-4.08 (m, 4H), 6.54 (dd, 1H, J₁=8.5 Hz, J₂=2.4 Hz), 6.72 (d, 1H, J=8.6 Hz), 6.80 (d, 1H, J=2.2 Hz), 7.25 (s, 1H), 8.02 (s, 1H). Following sulfonamide methylation and ester hydrolysis: ¹H NMR (400 MHz, CDCl₃) δ=1.46 (t, 3H, J=7.0 Hz), 1.47 (t, 3H, J=7.0 Hz), 3.30 (s, 3H), 4.04 (q, 2H, J=7.0 Hz), 4.11 (q, 2H, J=7.0 Hz), 6.56 (dd, 1H, J₁=8.6 Hz, J₂=2.5 Hz), 6.68 (d, 1H, J=2.5 Hz), 6.82 (d, 1H, J=8.6 Hz), 7.11 (s, 1H). Final step used ethyl 3-aminobenzoate. ¹H NMR (400 MHz, CDCl₃) δ=1.29 (t, 3H, J=7.0 Hz), 1.37 (t, 3H, J=7.0 Hz), 1.43 (t, 3H, J=7.0 Hz), 3.23 (s, 3H), 3.72 (q, 2H, J=7.0 Hz), 3.90 (q, 2H, J=7.0 Hz), 4.41 (q, 2H, J=7.0 Hz), 6.55-6.65 (m, 3H), 7.30 (s, 1H), 7.34 (t, 1H, J=8.0 Hz), 7.61 (ddd, 1H, J₁=8.1 Hz, J₂=2.2 Hz, J₃=1.0 Hz), 7.80 (ddd, 1H, J₁=7.9 Hz, J₂=2.5 Hz, J₃=1.2 Hz), 7.97 (t, 1H, J=1.9 Hz), 10.05 (s, 1H). ¹³C NMR (100 MHz, CDCl₃)=14.4, 14.5, 14.7, 38.9, 61.1, 64.4, 64.6, 112.5, 112.7, 119.1, 120.4, 123.7, 125.6, 128.7, 130.1, 131.1, 131.8, 132.0, 135.3, 137.4, 142.6, 149.1, 149.2, 156.5, 166.0. LC-MS (m/z): calc. for C₂₅H₂₈ClN₂O₇S₂ [M+H⁺]: 567.09, found: 567.0. HPLC purity: >99%. CL2-57 has an EC₅₀ in HepG2 liver cells of 1.8 μM.

Ethyl 3-chloro-5-(5-chloro-3-(N-(4-ethoxy-3-methoxyphenyl)-N-methylsulfamoyl)thiophene-2-carboxamido) benzoate (CL3-3). Reagents in first step, 4-ethoxy-3-methoxyaniline and 5, yielded methyl 5-chloro-3-(N-(4-ethoxy-3-methoxyphenyl)sulfamoyl)thiophene-2-carboxylate (7). 1H NMR (400 MHz, CDCl₃) δ=1.45 (t, 3H, J=7.0 Hz), 3.84 (s, 3H), 4.02 (s, 3H), 4.05 (q, 2H, J=7.0 Hz), 6.53 (dd, 1H, J₁=8.5 Hz, J₂=2.5 Hz), 6.72 (d, 1H, J=8.5 Hz), 6.81 (d, 1H, J=2.5 Hz), 7.26 (s, 1H), 8.05 (s, 1H). Following sulfonamide methylation: ¹H NMR (400 MHz, CDCl₃) δ=1.48 (t, 3H, J=7.0 Hz), 3.43 (s, 3H), 3.83 (s, 3H), 3.87 (s, 3H), 4.10 (q, 2H, J=7.0 Hz), 6.70 (dd, 1H, J₁=8.5 Hz, J₂=2.4 Hz), 6.78 (d, 1H, J=2.7 Hz), 6.79 (d, 1H, J=8.7 Hz), 7.04 (s, 1H). After ester hydrolysis: ¹H NMR (400 MHz, CDCl₃) δ=1.49 (t, 3H, J=7.0 Hz), 3.31 (s, 3H), 3.84 (s, 3H), 4.12 (q, 2H, J=6.9 Hz), 6.58 (dd, 1H, J₁=8.4 Hz, J₂=2.4 Hz), 6.68 (d, 1H, J=2.5 Hz), 6.82 (d, 1H, J=8.7 Hz), 7.12 (s, 1H). Final step used ethyl 3-amino-5-chlorobenzoate. ¹H NMR (400 MHz, CDCl₃) δ=1.34 (t, 1H, J=7.0 Hz), 1.43 (t, 3H, J=7.2 Hz), 3.23 (s, 3H), 3.74 (s, 3H), 3.77 (q, 2H, J=7.0 Hz), 4.41 (q, 2H, J=7.1 Hz), 6.56-6.58 (m, 2H), 6.69 (d, 1H, J=2.0 Hz), 7.37 (s, 1H), 7.71 (t, 1H, J=1.8 Hz), 7.74-7.76 (m, 2H). ¹³C NMR (100 MHz, CDCl₃) δ=14.3, 14.5, 38.9, 55.9, 61.5, 64.2, 111.1, 111.8, 118.3, 118.7, 123.2, 125.4, 130.2, 131.9, 132.0, 132.3, 134.7, 135.7, 138.4, 141.9, 148.9, 149.6, 156.7, 164.9. LC-MS (m/z): calc. for C₂₄H₂₅C₁₂N₂O₇S₂ [M+H⁺]: 587.0, found: 586.9. HPLC purity: 98%. CL3-3 has an EC₅₀ in HepG2 liver cells of 300 nM.

N-(3-acetylphenyl)-3-(N-(4-ethoxyphenyl)-N-methylsulfamoyl)thiophene-2-carboxamide (CL1-57). Synthesized via intermediate 3. Reagent in final step: 1-(3-aminophenyl)ethan-1-one. ¹H NMR (400 MHz, CDCl₃) δ=1.24 (t, 3H, J=7.0 Hz), 2.63 (s, 3H), 3.20 (s, 3H), 3.60 (q, 2H, J=7.0 Hz), 6.55-6.59 (m, 2H), 6.99-7.03 (m, 2H), 7.37 (t, 1H, J=7.9 Hz), 7.51-7.53 (m, 2H), 7.63 (d, 1H, J=5.3 Hz), 7.70-7.73 (m, 1H), 8.08 (t, 1H, J=2.1 Hz), 10.12 (s, 1H). HPLC purity: 97%.

3-(N-(4-ethoxyphenyl)-N-methylsulfamoyl)-N-(3-propionylphenyl)thiophene-2-carboxamide (CL1-62). Synthesized via intermediate 3. Reagent in final step: 1-(3-aminophenyl)propan-1-one. ¹H NMR (400 MHz, CDCl₃) δ=1.21-1.28 (m, 6H), 3.02 (q, 2H, J=7.2 Hz), 3.20 (s, 3H), 3.59 (q, 2H, J=7.0 Hz), 6.55-6.59 (m, 2H), 6.99-7.03 (m, 2H), 7.36 (t, 1H, J=8.0 Hz), 7.51-7.53 (m, 2H), 7.62 (d, 1H, J=5.3 Hz), 7.72 (dd, 1H, J₁=7.8 Hz, J₂=1.1 Hz), 8.08 (t, 1H, J=1.8 Hz), 10.12 (s, 1H). HPLC purity: 97%.

(3-(3-(N-(4-ethoxyphenyl)-N-methylsulfamoyl)thiophene-2-carboxamido)phenyl) boronic acid (CL1-86). Synthesized via intermediate 3. Reagent in final step: (3-aminophenyl)boronic acid. ¹H NMR (400 MHz, CDCl₃) δ=1.29 (t, 3H, J=7.0 Hz), 3.21 (s, 3H), 3.66 (q, 2H, J=6.9 Hz), 6.61 (dd, 2H, J₁=8.0 Hz, J₂=2.4 Hz), 7.01 (dd, 2H, J₁=8.1 Hz, J₂=2.4 Hz)), 7.33 (t, 1H, J=8.0 Hz), 7.41-7.43 (m, 2H), 7.49-7.52 (m, 1H), 7.61 (dd, 1H, J₁=5.4 Hz, J₂=3.8 Hz), 7.75 (s, 1H), 9.99 (s, 1H). HPLC purity: 93%.

Methyl 3-(3-(N-(4-ethoxyphenyl)-N-methylsulfamoyl)thiophene-2-carboxamido) benzoate (CL1-87). Synthesized via intermediate 3. Reagent in final step: methyl 3-aminobenzoate. ¹H NMR (400 MHz, CDCl₃) δ=1.26 (t, 3H, J=7.0 Hz), 3.19 (s, 3H), 3.61 (q, 2H, J=7.0 Hz), 3.95 (s, 3H), 6.55-6.58 (m, 2H), 6.99-7.02 (m, 2H), 7.35 (t, 1H, J=8.0 Hz), 7.50 (d, 1H, J=5.3 Hz), 7.59-7.62 (m, 2H), 7.79 (d, 1H, J=7.6 Hz), 8.04 (s, 1H), 10.09 (s, 1H). HPLC purity: 98%.

Isopropyl 3-(3-(N-(4-ethoxyphenyl)-N-methylsulfamoyl)thiophene-2-carboxamido) benzoate (CL1-88). Synthesized via intermediate 3. Reagent in final step: isopropyl 3-aminobenzoate. ¹H NMR (400 MHz, CDCl₃) δ=1.26 (t, 3H, J=7.0 Hz), 1.41 (s, 3H), 1.42 (s, 3H), 3.20 (s, 3H), 3.62 (q, 2H, J=7.0 Hz), 5.25-5.32 (m, 1H), 6.56-6.60 (m, 2H), 6.99-7.03 (m, 2H), 7.33 (t, 1H, J=8.0 Hz), 7.50 (d, 1H, J=5.4 Hz), 7.61-7.64 (m, 2H), 7.77 (ddd, 1H, J₁=6.9 Hz, J₂=1.6 Hz, J₃=1.2 Hz), 7.99 (s, 1H), 10.07 (s, 1H). HPLC purity: 94%.

N-(3-benzoylphenyl)-3-(N-(4-ethoxyphenyl)-N-methylsulfamoyl)thiophene-2-carboxamide (CLZ-117). Synthesized via intermediate 3. Reagent in final step: (3-aminophenyl) (phenyl)methanone. ¹H NMR (400 MHz, CDCl₃) δ=1.26 (t, 3H, J=7.0 Hz), 3.20 (s, 3H), 3.66 (q, 2H, J 7.0 Hz), 6.58-6.62 (m, 2H), 6.99-7.03 (m, 2H), 7.39 (t, 1H, J=6.9 Hz), 7.49-7.55 (m, 7H), 7.61-7.63 (m, 3H), 10.11 (s, 1H). HPLC purity: 90%.

Ethyl 3-(3-(N-(4-(2-methoxyethoxy)phenyl)-N-methylsulfamoyl)thiophene-2-carboxamido)benzoate (CL1-141). Reagents in first step: 4-(2-methoxyethoxy)aniline) and 1. Reagent in final step: ¹H NMR (400 MHz, CDCl₃) δ=1.43 (t, 3H, J=7.1 Hz), 3.20 (s, 3H), 3.41 (s, 3H), 3.58 (t, 2H, J=5.0 Hz), 3.72 (t, 2H, J=5.0 Hz), 4.42 (q, 2H, J=7.2 Hz), 6.64 (dd, 2H, J₁=6.8 Hz, J₂=2.2 Hz), 7.02 (dd, 2H, J=7.0 Hz, J₂=2.2 Hz), 7.35 (t, 1H, J=8.0 Hz), 7.48 (d, 1H, J=5.3 Hz), 7.60-7.65 (m, 2H), 7.78-7.80 (m, 1H), 8.04 (t, 1H, J=1.6 Hz), 10.10 (s, 1H). HPLC purity: 78%.

3-(N-(4-ethoxyphenyl)-N-methylsulfamoyl)-N-(3-(methylsulfonyl)phenyl)thiophene-2-carboxamide (CL1-159). Synthesized via intermediate 3. Reagent in final step: 3-(methylsulfonyl)aniline. ¹H NMR (400 MHz, CDCl₃) δ=1.27 (t, 3H, J=7.0 Hz), 3.08 (s, 3H), 3.20 (s, 3H), 3.67 (q, 2H, J=7.0 Hz), 6.60 (dd, 2H, J₁=6.7 Hz, J₂=2.2 Hz), 7.00-7.03 (m, 2H), 7.46 (t, 1H, J=8.0 Hz), 7.54 (d, 1H, J=5.3 Hz), 7.61-7.69 (m, 3H), 8.04 (t, 3H, J=2.3 Hz). HPLC purity: 91%.

Ethyl 3-(3-(N-(3-ethoxyphenyl)-N-methylsulfamoyl)thiophene-2-carboxamido)benzoate (CL2-1). Reagents in first step: 3-ethoxyaniline and 1. Final step used ethyl 3-aminobenzoate. ¹H NMR (400 MHz, CDCl₃) δ=1.29 (t, 3H, J=7.0 Hz), 1.44 (t, 3H, J=7.1 Hz), 3.21 (s, 3H), 3.72 (q, 2H, J=6.6 Hz), 4.41 (q, 2H, J=7.1 Hz), 6.47-6.50 (m, 1H), 6.58-6.60 (m, 1H), 6.72 (t, 1H, J=2.1 Hz), 7.05 (t, 1H, J=8.1 Hz), 7.34 (t, 1H, J=7.9 Hz), 7.49 (d, 1H, J=5.3 Hz), 7.61 (d, 1H, J=5.3 Hz), 7.64-7.67 (m, 1H), 7.80 (d, 1H, J=7.7 Hz), 8.03 (s, 1H), 10.10 (s, 1H). HPLC purity: 98%.

Ethyl 3-(3-(N-methyl-N-(4-propoxyphenyl)sulfamoyl)thiophene-2-carboxamido) benzoate (CL2-16). Reagents in first step: 4-propoxyaniline and 1. Final step used ethyl 3-aminobenzoate. ¹H NMR (400 MHz, DMSO-d₆) δ=0.97 (t, 3H, J=7.0 Hz), 1.34 (t, 3H, J=7.2 Hz), 1.61-1.68 (m, 2H), 3.15 (s, 3H), 3.77 (t, 2H, J=6.6 Hz), 4.34 (q, 2H, J=7.1 Hz), 6.79-6.82 (m, 2H), 7.00 (d, 1H, J=5.3 Hz), 7.05-7.09 (m, 2H), 7.49 (t, 1H, J=7.9 Hz), 7.70-7.75 (m, 2H), 7.88 (d, 1H, J=5.3 Hz), 8.22 (s, 1H), 10.62 (s, 1H). HPLC purity: 99%.

Ethyl 3-(3-(N-(3,4-diethoxyphenyl)-N-methylsulfamoyl)thiophene-2-carboxamido)benzoate (CL2-43). Reagents in first step: 3,4-diethoxyaniline and 1. Final step used ethyl 3-aminobenzoate. ¹H NMR (400 MHz, CDCl₃) δ=1.31 (t, 3H, J=7.0 Hz), 1.37 (t, 3H, J=7.0 Hz), 1.43 (t, 3H, J=7.0 Hz), 3.19 (s, 3H), 3.73 (q, 2H, J=7.0 Hz), 3.88 (q, 2H, J=7.0 Hz), 6.56-6.63 (m, 3H), 7.34 (t, 1H, J=8.0 Hz), 7.49 (d, 1H, J=5.3 Hz), 7.61 (d, 1H, J=5.3 Hz), 7.65 (ddd, 1H, J₁=8.2 Hz, J₂=2.2 Hz, J₃=1.0 Hz), 7.80 (ddd, 1H, J₁=7.8 Hz, J₂=1.5 Hz, J₃=1.1 Hz), 8.04 (t, 1H, J=2.2 Hz), 10.14 (s, 1H). HPLC purity: 94%.

Methyl 3-((3-(N-(3,4-diethoxyphenyl)-N-methylsulfamoyl)thiophene-2-carboxamido)methyl)benzoate (JB1-19). Synthesized via intermediate 3. Reagent in final step: ethyl 3-(aminomethyl)benzoate.

Methyl 3-(5-chloro-3-(N-(4-ethoxyphenyl)-N-methylsulfamoyl)thiophene-2-carboxamido)benzoate (KB7-123). Reagents in first step: 4-ethoxyaniline and 5. Final step used ethyl 3-aminobenzoate. ¹H NMR (400 MHz, CDCl₃) δ=1.23 (t, 3H, J=7.0 Hz), 1.44 (t, 3H, J=7.1 Hz), 3.23 (s, 3H), 3.60 (q, 2H, J=7.1 Hz), 6.58 (dd, 1H, J₁=6.8 Hz, J₂=2.2 Hz), 7.02-7.05 (m, 2H), 7.33 (t, 1H, J=7.9 Hz), 7.57 (ddd, 1H, J₁=8.1 Hz, J₂=2.2 Hz, J₃=1.0 Hz), 7.78 (ddd, 1H, J₁=7.8 Hz, J₂=1.6 Hz, J₃=1.1 Hz), 7.95 (t, 1H, J=1.8 Hz), 9.95 (s, 1H). HPLC purity: 97%.

5-Chloro-3-(N-(3,4-diethoxyphenyl)-N-methylsulfamoyl)-N-(3-(methylsulfonyl)phenyl)thiophene-2-carboxamide (CL2-63). Synthesized via intermediate 6. Reagent in final step: 3-(methylsulfonyl)aniline. ¹H NMR (400 MHz, CDCl₃) δ=1.259 (t, 3H, J=6.8 Hz), 1.264 (t, 3H, J=6.9 Hz), 3.04 (q, 2H, J=7.2 Hz), 3.23 (s, 3H), 3.67 (q, 2H, J=6.9 Hz), 3.90 (q, 2H, J=7.0 Hz), 6.51 (d, 1H, J=8.2 Hz), 6.63-6.66 (m, 2H), 7.32 (s, 1H), 7.36 (t, 1H, J=7.9 Hz), 7.50 (dd, 1H, J₁=8.0 Hz, J₂=1.2 Hz), 7.72 (d, 1H, J=7.8 Hz), 8.00 (s, 1H), 10.06 (s, 1H). HPLC purity: 94%.

5-Chloro-3-(N-(3,4-diethoxyphenyl)-N-methyl sulfamoyl)-N-(3-propionylphenyl)thiophene-2-carboxamide (CL2-64). Synthesized via intermediate 6. Reagent in final step: 1-(3-aminophenyl)propan-1-one. ¹H NMR (400 MHz, CDCl₃) δ=1.30 (t, 3H, J=7.0 Hz), 1.39 (t, 3H, J=7.0 Hz), 3.11 (s, 3H), 3.23 (s, 3H), 3.75 (q, 2H, J=7.0 Hz), 3.92 (q, 2H, J=7.0 Hz), 6.57-6.67 (m, 3H), 7.35 (s, 1H), 7.46 (t, 1H, J=7.9 Hz), 7.60 (dd, 1H, J₁=8.2 Hz, J₂=1.1 Hz), 7.67-7.69 (m, 1H), 7.96 (t, 1H, J=1.4 Hz). HPLC purity: 95%.

Ethyl 3-(5-chloro-3-(N-(4-ethoxy-3-methoxyphenyl)-N-methylsulfamoyl)thiophene-2-carboxamido)benzoate (CL2-85). Synthesized via intermediate 7. Final step used ethyl 3-aminobenzoate. ¹H NMR (400 MHz, CDCl₃) δ=1.30 (t, 3H, J=6.9 Hz), 1.43 (t, 3H, J=7.1 Hz), 3.23 (s, 3H), 3.717 (q, 2H, J=7.0 Hz), 3.716 (s, 3H), 4.41 (q, 2H, J=7.0 Hz), 6.54 (d, 1H, J=9.0 Hz), 6.63-6.65 (m, 2H), 7.33 (s, 1H), 7.34 (t, 1H, J=8.0 Hz), 7.62-7.65 (m, 1H), 7.79 (d, 1H, J=7.9 Hz), 7.92 (s, 1H), 10.01 (s, 1H). HPLC purity: 94%.

Ethyl 3-(5-chloro-3-(N-(4-ethoxy-2-fluorophenyl)-N-methylsulfamoyl)thiophene-2-carboxamido)benzoate (CL2-143). Reagents in first step: 4-ethoxy-2-fluoroaniline and 5. Final step used ethyl 3-aminobenzoate. ¹H NMR (400 MHz, CDCl₃) δ=1.27 (t, 3H, J=7.0 Hz), 1.44 (t, 3H, J=7.1 Hz), 3.28 (s, 3H), 3.61 (q, 2H, J=7.0 Hz), 4.42 (q, 2H, J=7.1 Hz), 6.35-6.42 (m, 2H), 7.19 (t, 1H, J=8.8 Hz), 7.34 (t, 1H, J=8.0 Hz), 7.35 (s, 1H), 7.63 (ddd, 1H, J₁=8.1 Hz, J₂=2.3 Hz, J₃=1.2 Hz), 7.78-7.81 (m, 1H), 7.96 (t, 1H, J=1.8 Hz), 10.15 (s, 1H). HPLC purity: 97%.

Ethyl 3-(5-chloro-3-(N-(6-ethoxypyxidin-3-yl)-N-methylsulfamoyl)thiophene-2-carboxamido)benzoate (CL2-159). Reagents in first step: 6-ethoxypyridin-3-amine and 5. Final step used ethyl 3-aminobenzoate. ¹H NMR (400 MHz, CDCl₃) δ=1.22 (t, 3H, J=7.0 Hz), 1.44 (t, 3H, J=7.2 Hz), 3.26 (s, 3H), 3.98 (q, 2H, J=7.0 Hz), 4.42 (q, 2H, J=7.2 Hz), 6.50 (d, 1H, J=8.6 Hz), 7.28-7.31 (m, 2H), 7.37 (t, 1H, J=8.0 Hz), 7.59-7.61 (m, 1H), 7.82 (d, 1H, J=8.0 Hz), 8.00-8.03 (m, 2H), 9.99 (s, 1H). HPLC purity: 91%.

2-Methoxyethyl 3-(5-chloro-3-(N-(3,4-diethoxyphenyl)-N-methylsulfamoyl)thiophene-2-carboxamido) benzoate (CL2-167). Synthesized from CL2-57 using 1-bromo-2-methoxyethane. ¹H NMR (400 MHz, CDCl₃) δ=1.29 (t, 3H, J=7.1 Hz), 1.36 (t, 3H, J=6.8 Hz), 3.23 (s, 3H), 3.47 (s, 3H), 3.70-3.78 (m, 4H), 3.89 (q, 2H, J=7.1 Hz), 4.49-4.51 (m, 2H), 6.56-6.64 (m, 3H), 7.30 (s, 1H), 7.40 (t, 1H, J=8.0 Hz), 7.66-7.68 (m, 1H), 7.81 (d, 1H, J=7.8 Hz), 7.94 (s, 1H), 10.05 (s, 1H). HPLC purity: 95%.

2-(2-Methoxyethoxy) ethyl 3-(5-chloro-3-(N-(3,4-diethoxyphenyl)-N-methylsulfamoyl)thiophene-2-carboxamido) benzoate (CL2-168). Synthesized from CL2-57 using 1-bromo-2-(2-methoxyethoxy)ethane). ¹H NMR (400 MHz, CDCl₃) δ=1.29 (t, 3H, J=7.0 Hz), 1.36 (t, 3H, J=7.0 Hz), 3.23 (s, 3H), 3.42 (s, 3H), 3.60-3.63 (m, 2H), 3.70-3.76 (m, 4H), 3.86-3.92 (m, 4H), 4.51 (t, 2H, J=4.8 Hz), 6.56-6.64 (m, 3H), 7.30 (s, 1H), 7.34 (t, 1H, J=8.0 Hz), 7.68 (dd, 1H, J₁=8.2 Hz, J₂=1.1 Hz), 7.81 (d, 1H, J=7.8 Hz), 7.93 (s, 1H), 10.06 (s, 1H). HPLC purity: 96%.

Ethyl 3-(5-chloro-3-(N-(4-(dimethylamino)phenyl)-N-methylsulfamoyl)thiophene-2-carboxamido)benzoate (CL2-181). Reagents in first step: N¹,N¹-dimethylbenzene-1,4-diamine and 5. Final step used ethyl 3-aminobenzoate. ¹H NMR (400 MHz, CDCl₃) δ=1.44 (t, 3H, J=7.2 Hz), 2.62 (s, 6H), 3.23 (s, 3H), 4.42 (q, 2H, J=7.2 Hz), 6.31 (d, 2H, J=9.0 Hz), 6.96 (d, 2H, J=9.0 Hz), 7.32 (t, 1H, J=8.0 Hz), 7.35 (s, 1H), 7.50-7.52 (m, 1H), 7.77 (d, 1H, J=8.1 Hz), 7.99 (t, 1H, J=1.6 Hz), 10.01 (s, 1H). HPLC purity: 94%.

Ethyl 3-(5-chloro-3-(N-(6-ethoxy-5-fluoropyridin-3-yl)-N-methylsulfamoyl)thiophene-2-carboxamido)benzoate (CL2-184). Reagents in first step: 6-ethoxy-5-fluoropyridin-3-amine and 5. Final step used ethyl 3-aminobenzoate. ¹H NMR (400 MHz, CDCl₃) δ=1.28 (t, 3H, J=7.0 Hz), 1.42 (t, 3H, J=6.9 Hz), 3.25 (s, 3H), 4.13 (q, 2H, J=7.2 Hz), 4.42 (q, 2H, J=7.2 Hz), 7.19 (dd, 1H, J₁=10.0 Hz, J₂=2.2 Hz), 7.30 (s, 1H), 7.40 (t, 1H, J=7.8 Hz), 7.64 (d, 1H, J=8.4 Hz), 7.75 (d, 1H, J=2.2 Hz), 7.84 (d, 1H, J=7.4 Hz), 8.03 (s, 1H), 9.93 (s, 1H). HPLC purity: 96%.

Ethyl 3-chloro-5-(5-chloro-3-(N-(3,4-diethoxyphenyl)-N-methylsulfamoyl)thiophene-2-carboxamido) benzoate (CL2-187). Synthesized via intermediate 6. Final step used ethyl 3-amino-5-chlorobenzoate. ¹H NMR (400 MHz, CDCl₃) δ=1.32 (t, 3H, J=7.0 Hz), 1.37 (t, 3H, J=7.0 Hz), 1.43 (t, 3H, J=7.2 Hz), 3.22 (s, 3H), 3.77 (q, 2H, J=7.0 Hz), 3.90 (q, 2H, J=7.0 Hz), 4.41 (q, 2H, J=7.2 Hz), 6.56-6.57 (m, 2H), 6.68 (d, 1H, J=2.2 Hz), 7.35 (s, 1H), 7.74-7.76 (m, 3H), 10.06 (s, 1H). HPLC purity: 97%.

Ethyl 3-amino-5-(5-chloro-3-(N-(3,4-diethoxyphenyl)-N-methylsulfamoyl)thiophene-2-carboxamido)benzoate (CL2-191). Synthesized via intermediate 6. Reagent in final step: ethyl 3,5-diaminobenzoate. ¹H NMR (400 MHz, CDCl₃) δ=1.33 (t, 3H, J=7.1 Hz), 1.38 (t, 3H, J=7.0 Hz), 1.41 (t, 3H, J=7.2 Hz), 3.23 (s, 3H), 3.81 (q, 2H, J=7.0 Hz), 3.92 (q, 2H, J=6.9 Hz), 4.37 (q, 2H, J=7.2 Hz), 6.61 (s, 2H), 6.67 (s, 1H), 7.06-7.11 (m, 2H), 7.26-7.28 (m, 2H), 9.92 (s, 1H). HPLC purity: 89%.

Ethyl 3-(5-chloro-3-(N-methyl-N-(4-(piperazin-1-yl)phenyl) sulfamoyl)thiophene-2-carboxamido)benzoate (CL2-200). Reagents in first step: 4-(piperazin-1-yl)aniline and 5. Final step used reagent ethyl 3-aminobenzoate. ¹H NMR (400 MHz, CDCl₃) δ=0.94 (t, 4H, J=7.1 Hz), 1.43 (t, 3H, J=7.1 Hz), 3.01 (q, 4H, J=7.1 Hz), 3.20 (s, 3H), 4.41 (q, 2H, J=7.1 Hz), 6.30 (dd, 2H, J₁=7.0 Hz, J₂=2.3 Hz), 6.93 (dd, 2H, J₁=6.9 Hz, J₂=2.3 Hz), 7.29 (t, 1H, J=8.0 Hz), 7.34 (s, 1H), 7.54 (ddd, 1H, J₁=8.2 Hz, J₂=2.3 Hz, J₃=1.2 Hz), 7.74-7.77 (m, 1H), 8.04 (t, 1H, J=1.8 Hz), 10.09 (s, 1H). HPLC purity: 98%.

Ethyl 6-(5-chloro-3-(N-(3,4-diethoxyphenyl)-N-methylsulfamoyl)thiophene-2-carboxamido)picolinate (CL3-2). Synthesized via intermediate 6. Reagent in final step: ethyl 6-aminopicolinate. ¹H NMR (400 MHz, CDCl₃) δ=1.43 (t, 3H, J=7.1 Hz), 1.44 (t, 3H, J=7.0 Hz), 1.46 (t, 3H, J=7.0 Hz), 3.33 (s, 3H), 4.03 (q, 2H, J=7.0 Hz), 4.09 (q, 2H, J=7.0 Hz), 4.46 (q, 2H, J=7.1 Hz), 6.64 (dd, 1H, J₁=7.0 Hz, J₂=2.2 Hz), 6.71-6.73 (m, 2H), 6.81 (d, 1H, J=8.6 Hz), 7.05 (s, 1H), 7.50 (d, 1H, J=7.0 Hz), 7.56 (d, 1H, J=8.1 Hz), 10.10 (s, 1H). HPLC purity: 93%.

Ethyl 3-chloro-5-(5-chloro-3-(N-(3,4-dimethoxyphenyl)-N-methylsulfamoyl)thiophene-2-carboxamido)benzoate (CL3-9). Reagents in first step: 3,4-dimethoxyaniline and 5. Final step used ethyl 3-amino-5-chlorobenzoate. ¹H NMR (400 MHz, CDCl₃) δ=1.43 (t, 3H, J=7.2 Hz), 3.23 (s, 3H), 3.63 (s, 3H), 3.74 (s, 3H), 4.40 (q, 2H, J=7.1 Hz), 6.57-6.63 (m, 2H), 6.68 (d, 1H, J=2.1 Hz), 7.37 (s, 1H), 7.70-7.75 (m, 3H), 10.00 (s, 1H). HPLC purity: 95%.

Methyl 3-chloro-5-(5-chloro-3-(N-(4-ethoxy-3-methoxyphenyl)-N-methylsulfamoyl)thiophene-2-carboxamido)benzoate (CL3-10). Synthesized via intermediate 7. Reagent in final step: methyl 3-amino-5-chlorobenzoate. ¹H NMR (400 MHz, CDCl₃) δ=1.33 (t, 3H, J=7.0 Hz), 3.23 (s, 3H), 3.74 (s, 3H), 3.77 (t, 2H, J=7.0 Hz), 3.95 (s, 3H), 6.53-6.59 (m, 2H), 6.69 (d, 1H, J=1.8 Hz), 7.37 (s, 1H), 7.70 (s, 1H), 7.74 (s, 1H), 7.75 (s, 1H), 10.03 (s, 1H). HPLC purity: 95%.

2-Methoxyethyl 3-chloro-5-(5-chloro-3-(N-(4-ethoxy-3-methoxyphenyl)-N-methylsulfamoyl)thiophene-2-carboxamido)benzoate (CL3-11). Synthesized from CL3-3 using 1-bromo-2-methoxyethane. ¹H NMR (400 MHz, CDCl₃) δ=1.34 (t, 3H, J=7.0 Hz), 3.23 (s, 3H), 3.46 (s, 3H), 3.73 (s, 3H), 3.74-3.78 (m, 4H), 4.50 (t, 2H, J=5.0 Hz), 6.55-6.69 (m, 2H), 6.67 (d, 1H, J=1.1 Hz), 7.36 (s, 1H), 7.69 (d, 1H, J=1.9 Hz), 7.75 (d, 1H, J=1.9 Hz), 7.80 (t, 1H, J 1.9 Hz), 10.03 (s, 1H). HPLC purity: 94%.

2-(2-Methoxyethoxy) ethyl 3-chloro-5-(5-chloro-3-(N-(4-ethoxy-3-methoxyphenyl)-N-methylsulfamoyl)thiophene-2-carboxamido)benzoate (CL3-12). Synthesized from CL3-3 using 1-bromo-2-(2-methoxyethoxy)ethane). ¹H NMR (400 MHz, CDCl₃) δ 1.33 (t, 3H, J=7.0 Hz), 3.23 (s, 3H), 3.43 (s, 3H), 3.62 (q, 2H, J=6.9 Hz), 3.73 (s, 3H), 3.75 (t, 2H, J=7.0 Hz), 3.78 (t, 2H, J=7.0 Hz), 3.87 (t, 2H, J=4.9 Hz), 4.51 (t, 2H, J=5.0 Hz), 6.55-6.58 (m, 2H), 6.68 (d, 1H, J=2.0 Hz), 7.36 (s, 1H), 7.67 (t, 1H, J=1.6 Hz), 7.76 (t, 1H, J=1.6 Hz), 7.81 (t, 1H, J=2.0 Hz), 10.03 (s, 1H). HPLC purity: 96%.

Ethyl 3-(5-chloro-3-(N-(4-ethoxy-3-methoxyphenyl)-N-methylsulfamoyl)thiophene-2-carboxamido)-5-fluorobenzoate (CL3-14). Synthesized via intermediate 7. Reagent in final step: ethyl 3-amino-5-fluorobenzoate. ¹H NMR (400 MHz, CDCl₃) δ=1.33 (t, 3H, J=7.0 Hz), 1.43 (t, 3H, J=7.1 Hz), 3.23 (s, 3H), 3.72 (s, 3H), 3.76 (q, 2H, J=7.1 Hz), 4.41 (q, 2H, J=7.2 Hz), 6.56-6.58 (m, 2H), 6.67 (d, 1H, J=2.2 Hz), 7.36 (s, 1H), 7.45-7.48 (m, 1H), 7.53 (s, 1H), 7.58-7.61 (m, 1H), 10.05 (s, 1H). HPLC purity: 93%.

### Synthesis of GR1-001 (Scheme 3).

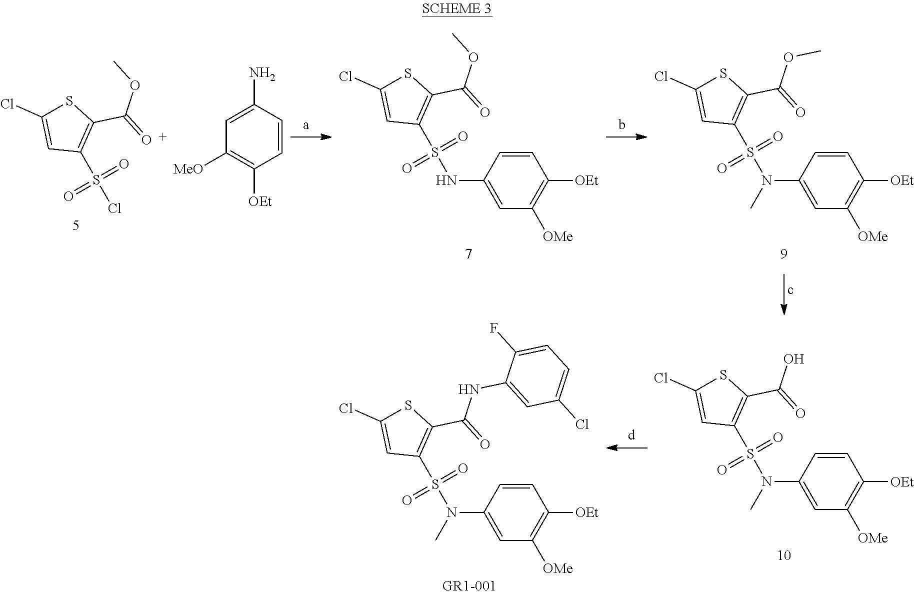 [SMILES: COC(=O)c1sc(Cl)cc1S(=O)(=O)Cl ; CCOc1ccc(N)cc1OC ; CCOc1ccc(NS(=O)(=O)c2cc(Cl)sc2C(=O)OC)cc1OC ; CCOc1ccc(N(C)S(=O)(=O)c2cc(Cl)sc2C(=O)OC)cc1OC ; CCOc1ccc(N(C)S(=O)(=O)c2cc(Cl)sc2C(=O)Nc2cc(Cl)ccc2F)cc1OC ; CCOc1ccc(N(C)S(=O)(=O)c2cc(Cl)sc2C(=O)O)cc1OC]

Conditions: (a) TEA, THF, rt, 24 h; (b) NaH, MeI, DMF, 0° C.-rt, 24 h; (c) THE-H₂O, NaOH, rt, Overnight; (d) 5-chloro-2-fluoroaniline, HATU, DMAP, DMF, 0° C.-rt, 8 h.

General Procedure for Acid-Amine Coupling Reaction. To a solution of carboxylic acid (10, 1 mmol) and HATU (1.2 mmol) in anhydrous DMF was added DMAP (1.2 mmol) at 0° C. under a nitrogen atmosphere. The reaction mixture was stirred for 10 minutes, substituted arylamines (1.0 mmol) were added and the reaction was stirred at room temperature for an additional 8 hours. The reaction mixture was filtered and directly injected to prep-HPLC to give title compounds in 80-90% yield.

5-Chloro-N-(5-chloro-2-fluorophenyl)-3-(N-(4-ethoxy-3-methoxyphenyl)-N-methylsulfamoyl)thiophene-2-carboxamide (GR1-001). Reactant in final step (d): 5-chloro-2-fluoroaniline. ¹H NMR (500 MHz, CDCl₃) δ 9.93 (s, 1H), 8.13 (dd, J=6.9, 2.5 Hz, 1H), 7.19 (s, 1H), 6.96-6.87 (m, 2H), 6.63 (t, J=1.3 Hz, 1H), 6.48 (d, J=1.4 Hz, 2H), 3.74-3.69 (m, 2H), 3.69 (s, 3H), 3.14 (s, 3H), 1.29 (t, J=7.0 Hz, 3H). ¹³C NMR (126 MHz, CDCl₃) δ 156.79, 151.75, 149.79, 149.25, 148.87, 141.78, 135.83, 131.90, 131.86, 130.12, 129.37, 129.34, 126.79, 126.70, 124.45, 124.39, 121.10, 118.54, 115.76, 115.59, 111.82, 111.50, 111.49, 64.22, 55.70, 38.71, 14.56. Molecular formula: C₂₁H₁₉Cl₂FN₂O₅S₂. Molecular weight: 533.41.

N-(3,5-bis (trifluoromethyl)phenyl)-5-chloro-3-(N-(4-ethoxy-3-methoxyphenyl)-N-methylsulfamoyl)thiophene-2-carboxamide (GR1-002). Reactant in final step (d): 3,5-bis(trifluoromethyl)aniline. ¹H NMR (500 MHz, CDCl₃) δ 10.12 (s, 1H), 7.68-7.64 (m, 2H), 7.45-7.42 (m, 1H), 7.13 (s, 1H), 6.51 (d, J=2.5 Hz, 1H), 6.46 (dd, J=8.5, 2.5 Hz, 1H), 6.39 (d, J=8.5 Hz, 1H), 3.57 (s, 3H), 3.54 (t, J=7.0 Hz, 2H), 3.09 (s, 3H), 1.13 (t, J=7.0 Hz, 3H). ¹³C NMR (126 MHz, CDCl₃) δ 156.94, 149.68, 149.01, 141.23, 138.65, 136.26, 132.56, 132.33, 132.29, 132.02, 131.87, 131.76, 130.29, 124.06, 121.89, 119.01, 118.82, 117.67, 111.89, 111.02, 64.16, 55.86, 38.93, 14.23. Molecular formula: C₂₃H₁₉ClF₆N₂O₅S₂. Molecular weight: 616.97.

5-Chloro-3-(N-(4-ethoxy-3-methoxyphenyl)-N-methylsulfamoyl)-N-(4-(methylsulfonyl)phenyl)thiophene-2-carboxamide (GR1-003). Reactant in final step (d): 4-(methylsulfonyl)aniline. ¹H NMR (500 MHz, CDCl₃) δ 10.06 (s, 1H), 7.72-7.68 (m, 2H), 7.41-7.37 (m, 2H), 7.16 (s, 1H), 6.53 (dd, J=8.5, 2.5 Hz, 1H), 6.43 (d, J=2.5 Hz, 1H), 6.35 (d, J=8.6 Hz, 1H), 3.61 (s, 3H), 3.57 (q, J=6.9 Hz, 2H), 3.10 (s, 3H), 2.94 (s, 3H), 1.22 (t, J=7.0 Hz, 3H). ¹³C NMR (126 MHz, CDCl₃) δ 156.87, 149.40, 148.86, 141.94, 141.57, 136.00, 135.70, 132.39, 131.77, 130.27, 128.29, 119.64, 119.35, 112.04, 110.28, 64.21, 55.76, 44.65, 38.93, 29.69, 14.55. Molecular formula: C₂₂H₂₃ClN₂O₇S₃. Molecular weight: 559.06.

5-Chloro-3-(N-(4-ethoxy-3-methoxyphenyl)-N-methylsulfamoyl)-N-(4-morpholinophenyl)thiophene-2-carboxamide (GR1-004). Reactant in final step (d): 4-morpholinoaniline. ¹H NMR (500 MHz, CDCl₃) δ 9.80 (s, 1H), 7.25 (d, J=9.2 Hz, 2H), 6.77 (d, J=8.5 Hz, 2H), 6.64 (dd, J=8.5, 2.5 Hz, 1H), 6.55 (d, J=2.5 Hz, 1H), 6.53 (d, J=8.6 Hz, 1H), 3.84 (t, J=4.8 Hz, 4H), 3.74 (q, J=7.0 Hz, 2H), 3.71 (s, 3H), 3.18 (s, 3H), 3.09 (dd, J=5.8, 3.8 Hz, 4H), 1.31 (t, J=7.0 Hz, 3H). ¹³C NMR (126 MHz, CDCl₃) δ 155.84, 149.48, 148.80, 143.37, 134.54, 132.10, 131.39, 130.02, 120.65, 119.66, 115.75, 112.24, 110.52, 66.84, 64.21, 55.91, 49.55, 38.90, 29.69, 14.66. Molecular formula: C₂₅H₂₈ClN₃O₆S₂. Molecular weight: 566.08.

N-(4-(tert-butyl)phenyl)-5-chloro-3-(N-(4-ethoxy-3-methoxyphenyl)-N-methylsulfamoyl)thiophene-2-carboxamide (GR1-005). Reactant in final step (d): 4-(tert-butyl)aniline. ¹H NMR (500 MHz, CDCl₃) δ 9.84 (s, 1H), 7.24 (s, 2H), 7.20 (s, 1H), 6.65 (dd, J=8.6, 2.5 Hz, 1H), 6.51 (dd, J=5.5, 3.0 Hz, 2H), 3.68 (d, J=7.5 Hz, 5H), 3.18 (s, 3H), 1.29 (d, J=7.0 Hz, 3H), 1.27 (d, J=1.6 Hz, 9H). ¹³C NMR (126 MHz, CDCl₃) δ 156.12, 149.53, 148.85, 147.59, 143.36, 134.71, 134.62, 132.03, 131.36, 130.01, 125.47, 119.82, 119.32, 112.44, 110.45, 64.18, 55.93, 38.92, 34.37, 31.32, 14.63. Molecular formula: C₂₅H₂₉ClN₂O₅S₂. Molecular weight: 537.09.

5-Chloro-N-(3-chloro-4-(trifluoromethyl)phenyl)-3-(N-(4-ethoxy-3-methoxyphenyl)-N-methylsulfamoyl)thiophene-2-carboxamide (GR1-006). Reactant in final step (d): 3-chloro-4-(trifluoromethyl)aniline ¹H NMR (500 MHz, CDCl₃) δ 9.94 (s, 1H), 7.41 (d, J=8.6 Hz, 1H), 7.39 (d, J=2.1 Hz, 1H), 7.18-7.15 (m, 1H), 6.54 (dd, J=8.5, 2.5 Hz, 1H), 6.44 (d, J=2.5 Hz, 1H), 6.37 (d, J=8.5 Hz, 1H), 3.62 (s, 3H), 3.56 (q, J=7.0 Hz, 2H), 3.09 (s, 3H), 1.19 (t, J=7.0 Hz, 3H). ¹³C NMR (126 MHz, CDCl₃) δ 156.84, 149.56, 149.02, 141.43, 140.97, 136.08, 132.71, 132.69, 132.37, 131.74, 130.30, 127.79, 127.75, 123.94, 123.85, 123.68, 121.68, 121.41, 119.63, 116.78, 111.93, 110.32, 64.21, 55.89, 38.85, 29.69, 14.37. Molecular formula: C₂₂H₁₉C₁₂F₃N₂O₅S₂. Molecular weight: 583.42.

5-Chloro-N-(2-chloro-5-(trifluoromethyl)phenyl)-3-(N-(4-ethoxy-3-methoxyphenyl)-N-methylsulfamoyl)thiophene-2-carboxamide (GR1-007). Reactant in final step (d): 2-chloro-5-(trifluoromethyl)aniline. ¹H NMR (500 MHz, CDCl₃) δ 9.95 (s, 1H), 8.32 (d, J=2.1 Hz, 1H), 7.38 (d, J=8.3 Hz, 1H), 7.24-7.21 (m, 1H), 6.63 (t, J=1.3 Hz, 1H), 6.51 (d, J=1.4 Hz, 2H), 3.71 (q, J=7.0 Hz, 2H), 3.64 (s, 3H), 3.16 (s, 3H), 1.26 (t, J=7.0 Hz, 4H). ¹³C NMR (126 MHz, CDCl₃) δ 157.39, 149.32, 148.77, 141.55, 136.07, 134.84, 132.25, 132.01, 130.04, 129.89, 129.68, 129.42, 127.25, 124.57, 122.40, 121.88, 121.85, 121.82, 119.30, 119.27, 119.24, 118.73, 111.54, 111.43, 64.11, 55.82, 38.78, 29.69, 14.30. Molecular formula: C₂₂H₁₉C₁₂F₃N₂O₅S₂. Molecular weight: 583.42.

5-Chloro-3-(N-(4-ethoxy-3-methoxyphenyl)-N-methylsulfamoyl)-N-(4-(2-oxopyrrolidin-1-yl)phenyl)thiophene-2-carboxamide (GR1-009). Reactant in final step (d): 1-(4-aminophenyl)pyrrolidin-2-one. ¹H NMR (500 MHz, CDCl₃) δ 9.92 (s, 1H), 7.54-7.49 (m, 2H), 7.37-7.33 (m, 2H), 6.65 (dd, J=8.5, 2.5 Hz, 1H), 6.56-6.52 (m, 2H), 3.83 (t, J=7.0 Hz, 2H), 3.75 (q, J=7.0 Hz, 2H), 3.72 (s, 3H), 3.20 (s, 3H), 2.60 (t, J=8.1 Hz, 2H), 2.16 (tt, J=7.8, 6.8 Hz, 2H), 1.30 (t, J=7.0 Hz, 3H). ¹³C NMR (126 MHz, CDCl₃) δ 173.94, 156.15, 149.44, 148.80, 142.96, 136.05, 134.88, 133.59, 131.97, 131.67, 130.08, 119.94, 119.80, 119.68, 112.19, 110.40, 64.21, 55.90, 48.70, 38.89, 32.67, 29.69, 17.93, 14.58. Molecular formula: C₂₅H₂₆ClN₃O₆S₂. Molecular weight: 564.07.

5-Chloro-3-(N-(4-ethoxy-3-methoxyphenyl)-N-methylsulfamoyl)-N-(4-(trifluoromethyl)phenyl)thiophene-2-carboxamide (GR1-010). Reactant in final step (d): 4-(trifluoromethyl)aniline. ¹H NMR (500 MHz, CDCl₃) δ 9.95 (s, 1H), 7.40 (d, J=8.7 Hz, 2H), 7.34 (d, J=8.6 Hz, 2H), 6.60 (dd, J=8.5, 2.5 Hz, 1H), 6.42 (d, J=2.5 Hz, 1H), 6.34 (d, J=8.6 Hz, 1H), 3.64 (s, 3H), 3.51 (q, J=7.0 Hz, 2H), 3.12 (s, 3H), 1.19 (s, 3H). ¹³C NMR (126 MHz, CDCl₃) δ 156.70, 149.46, 148.90, 142.08, 140.24, 135.65, 132.14, 131.78, 130.23, 126.37, 126.11, 125.83, 125.80, 125.76, 125.09, 122.93, 120.02, 119.20, 112.05, 109.98, 64.10, 55.85, 38.85, 29.69, 14.35. Molecular formula: C₂₂H₂₀ClF₃N₂O₅S₂. Molecular weight: 548.98.

Ethyl 4-(5-chloro-3-(N-(4-ethoxy-3-methoxyphenyl)-N-methylsulfamoyl)thiophene-2-carboxamido)benzoate (GR1-011). Reactant in final step (d): ethyl 4-aminobenzoate. ¹H NMR (500 MHz, CDCl₃) δ 9.96 (s, 1H), 7.88-7.81 (m, 2H), 7.31 (d, J=8.8 Hz, 2H), 6.57 (dd, J=8.6, 2.5 Hz, 1H), 6.47 (d, J=2.5 Hz, 1H), 6.38 (d, J=8.7 Hz, 1H), 4.29 (q, J=7.1 Hz, 2H), 3.65 (s, 3H), 3.56 (q, J=7.0 Hz, 2H), 3.13 (s, 3H), 1.32 (t, J=7.1 Hz, 3H), 1.20 (t, J=7.0 Hz, 3H). 13C NMR (126 MHz, CDCl₃) δ 165.95, 156.60, 149.47, 148.88, 142.30, 141.17, 135.51, 132.07, 131.83, 130.34, 130.20, 126.27, 119.71, 118.74, 112.06, 110.27, 77.25, 77.00, 76.74, 64.15, 60.84, 55.85, 38.84, 14.43, 14.34. Molecular formula: C₂₄H₂₅ClN₂O₇S₂. Molecular weight: 553.04.

Ethyl 2-chloro-4-(5-chloro-3-(N-(4-ethoxy-3-methoxyphenyl)-N-methylsulfamoyl)thiophene-2-carboxamido)benzoate (GR1-012). Reactant in final step (d): ethyl 4-amino-2-chlorobenzoate. ¹H NMR (500 MHz, CDCl₃) δ 9.92 (s, 1H), 7.67 (d, J=8.6 Hz, 1H), 7.33 (d, J=2.1 Hz, 1H), 7.18 (d, J=2.1 Hz, 1H), 7.16 (d, J=1.1 Hz, 1H), 6.53 (dd, J=8.5, 2.5 Hz, 1H), 6.47 (d, J=2.5 Hz, 1H), 6.40 (d, J=8.5 Hz, 1H), 4.28 (q, J=7.1 Hz, 2H), 3.64 (s, 3H), 3.61 (q, J=7.0 Hz, 2H), 3.11 (s, 3H), 1.30 (t, J=7.1 Hz, 3H), 1.22 (t, J=7.0 Hz, 3H). ¹³C NMR (126 MHz, CDCl₃) δ 164.88, 156.72, 149.58, 149.03, 141.75, 140.71, 135.89, 134.70, 132.26, 132.19, 131.77, 130.25, 125.36, 121.18, 119.52, 116.88, 112.00, 110.47, 77.26, 77.00, 76.75, 64.28, 61.33, 55.92, 38.86, 14.48, 14.23. Molecular formula: C₂₄H₂₄Cl₂N₂O₇S₂. Molecular weight: 586.04.

5-Chloro-N-(3-chlorophenyl)-3-(N-(4-ethoxy-3-methoxyphenyl)-N-methylsulfamoyl)thiophene-2-carboxamide (GR1-046). Reactant in final step (d): 3-chloroaniline. ¹H NMR (500 MHz, CDCl₃) δ 9.83 (s, 1H), 7.37 (t, J=2.1 Hz, 1H), 7.11-7.05 (m, 2H), 6.98 (dt, J=7.1, 2.0 Hz, 1H), 6.53 (q, J=3.3 Hz, 2H), 6.47 (d, J=9.2 Hz, 1H), 3.72-3.67 (m, 2H), 3.66 (s, 3H), 3.14 (s, 3H), 1.26 (t, J=7.0 Hz, 3H). ¹³C NMR (126 MHz, CDCl₃) δ 156.43, 149.58, 148.98, 142.44, 138.33, 135.36, 134.42, 131.91, 131.82, 130.14, 129.60, 124.52, 119.45, 119.21, 117.38, 112.08, 110.84, 64.27, 55.91, 38.88, 14.55. Molecular formula: C₂₁H₂₀C₁₂N₂O₅S₂. Molecular weight: 515.42.

5-Chloro-3-(N-(4-ethoxy-3-methoxyphenyl)-N-methylsulfamoyl)-N-(3-fluorophenyl)thiophene-2-carboxamide (GR1-047). Reactant in final step (d): 3-fluoroaniline. ¹H NMR (500 MHz, CDCl₃) δ 9.87 (s, 1H), 7.20 (d, J=1.5 Hz, 1H), 7.12 (td, J=8.2, 6.3 Hz, 1H), 6.89-6.85 (m, 1H), 6.75-6.69 (m, 1H), 6.56-6.52 (m, 2H), 6.47 (d, J=8.2 Hz, 1H), 3.70 (t, J=7.0 Hz, 2H), 3.66 (s, 3H), 3.15 (s, 3H), 1.27 (t, J=7.0 Hz, 3H). ¹³C NMR (126 MHz, CDCl₃) δ 162.73 (d, J_{CF}=244.7 Hz), 156.44, 149.54, 148.96, 142.49, 138.65 (d, J_{CF}=10.8 Hz), 135.30, 131.90, 131.79, 130.13, 129.69 (d, J_{CF}=9.4 Hz), 119.24, 114.83 (d, J_{CF}=3.1 Hz), 112.05, 111.31, 111.13, 110.77, 107.06, 106.85, 64.22, 55.84, 38.88, 14.47. Molecular formula: C₂₁H₂₀ClFN₂O₅S₂. Molecular weight: 498.96.

N-(3-bromophenyl)-5-chloro-3-(N-(4-ethoxy-3-methoxyphenyl)-N-methylsulfamoyl)thiophene-2-carboxamide (GR1-048). Reactant in final step (d): 3-bromoaniline. ¹H NMR (500 MHz, CDCl₃) δ 9.83 (s, 1H), 7.51 (t, J=2.0 Hz, 1H), 7.15-7.11 (m, 2H), 7.03 (t, J=8.0 Hz, 1H), 6.54 (d, J=7.1 Hz, 2H), 6.48-6.45 (m, 1H), 3.70 (t, J=7.0 Hz, 2H), 3.67 (s, 3H), 3.14 (s, 3H), 1.27 (t, J=7.0 Hz, 3H). ¹³C NMR (126 MHz, CDCl₃) δ 156.41, 149.59, 148.98, 142.41, 138.46, 135.37, 131.91, 131.83, 130.14, 129.89, 127.45, 122.38, 122.23, 119.22, 117.83, 112.10, 110.83, 64.30, 55.95, 38.88, 14.60. Molecular formula: C₂₁H₂₀BrClN₂O₅S₂. Molecular weight: 559.87.

5-Chloro-N-(3,5-dichlorophenyl)-3-(N-(4-ethoxy-3-methoxyphenyl)-N-methylsulfamoyl)thiophene-2-carboxamide (GR1-049). Reactant in final step (d): 3,5-dichloroaniline. ¹H NMR (500 MHz, CDCl₃) δ 9.90 (s, 1H), 7.33 (s, 1H), 7.24 (s, 1H), 7.06 (t, J=1.8 Hz, 1H), 6.64 (d, J=2.0 Hz, 1H), 6.54 (d, J=2.9 Hz, 2H), 3.79 (q, J=7.0 Hz, 2H), 3.75 (s, 3H), 3.19 (s, 3H), 1.35 (t, J=7.0 Hz, 3H). ¹³C NMR (126 MHz, CDCl₃) δ 156.58, 149.66, 149.05, 141.81, 138.94, 135.79, 134.99, 131.97, 131.84, 130.21, 124.33, 118.72, 117.49, 111.86, 111.05, 64.32, 55.95, 38.88, 14.55. Molecular formula: C₂₁H₁₉Cl₃N₂O₅S₂. Molecular weight: 549.86.

5-Chloro-N-(3-chloro-5-fluorophenyl)-3-(N-(4-ethoxy-3-methoxyphenyl)-N-methylsulfamoyl)thiophene-2-carboxamide (GR1-050). Reactant in final step (d): 3-chloro-5-fluoroaniline. ¹H NMR (500 MHz, CDCl₃) δ 9.84 (s, 1H), 7.01 (dt, J=10.5, 2.2 Hz, 1H), 6.93 (q, J=1.6 Hz, 1H), 6.71 (dt, J=8.3, 2.1 Hz, 1H), 6.56-6.53 (m, 1H), 6.46 (d, J=1.8 Hz, 2H), 3.70 (q, J=7.0 Hz, 2H), 3.65 (s, 3H), 3.10 (d, J=1.6 Hz, 3H), 1.26 (t, J=7.0 Hz, 3H). ¹³C NMR (126 MHz, CDCl₃) δ 162.55 (d, J=247.6 Hz), 156.59, 149.62, 149.03, 141.83, 139.17 (d, J=12.3 Hz), 135.75, 134.97 (d, J=12.4 Hz), 131.97, 131.83, 130.20, 118.78, 115.05, 115.03, 112.08, 111.87 (d, J=3.1 Hz), 110.98, 105.23, 105.02, 64.26, 55.86, 38.88, 14.48. Molecular formula: C₂₁H₁₉Cl₂FN₂O₅S₂. Molecular weight: 533.41.

N-(3-bromo-5-chlorophenyl)-5-chloro-3-(N-(4-ethoxy-3-methoxyphenyl)-N-methylsulfamoyl)thiophene-2-carboxamide (GR1-051). Reactant in final step (d): 3-bromo-5-chloroaniline. ¹H NMR (500 MHz, CDCl₃) δ 9.80 (s, 1H), 7.28 (d, J=1.8 Hz, 1H), 7.20 (t, J=1.9 Hz, 1H), 7.11 (t, J=1.7 Hz, 1H), 6.55 (d, J=2.1 Hz, 1H), 6.48-6.42 (m, 2H), 3.70 (q, J=7.0 Hz, 2H), 3.66 (s, 3H), 3.10 (s, 3H), 1.26 (t, J=7.0 Hz, 3H). ¹³C NMR (126 MHz, CDCl₃) δ 156.55, 149.67, 149.05, 141.79, 139.09, 135.80, 135.13, 131.97, 131.84, 130.21, 127.09, 122.51, 120.26, 118.74, 117.96, 111.89, 111.04, 64.34, 56.01, 38.88, 14.59. Molecular formula: C₂₁H₁₉BrCl₂N₂O₅S₂. Molecular weight: 594.31.

5-Chloro-N-(3-chloro-5-(trifluoromethyl)phenyl)-3-(N-(4-ethoxy-3-methoxyphenyl)-N-methylsulfamoyl)thiophene-2-carboxamide (GR1-052). Reactant in final step (d): 3-chloro-5-(trifluoromethyl)aniline. ¹H NMR (500 MHz, CDCl₃) δ 9.96 (s, 1H), 7.42 (d, J=2.0 Hz, 1H), 7.36 (d, J=1.9 Hz, 1H), 7.19 (d, J=1.8 Hz, 1H), 6.53 (d, J=2.3 Hz, 1H), 6.47-6.40 (m, 2H), 3.63 (t, J=7.0 Hz, 2H), 3.61 (s, 3H), 3.09 (s, 3H), 1.19 (t, J=7.0 Hz, 3H). ¹³C NMR (126 MHz, CDCl₃) δ 156.75, 149.68, 149.03, 141.54, 138.88, 136.01, 135.18, 132.80, 132.53, 132.27, 132.15, 131.86, 130.24, 124.07, 122.15, 121.91, 121.14-121.08 (m, C—CF₃), 118.88, 114.12, 114.09, 114.06, 114.03, 111.88, 111.04, 64.24, 55.90, 38.90, 14.39. Molecular formula: C₂₂H₁₉Cl₂F₃N₂O₅S₂. Molecular weight: 583.41.

Ethyl 3-(5-chloro-3-(N-(4-ethoxy-3-methoxyphenyl)-N-methylsulfamoyl)thiophene-2-carboxamido)-5-fluorobenzoate (GR1-053). Reactant in final step (d): ethyl 3-amino-5-fluorobenzoate. ¹H NMR (500 MHz, CDCl₃) δ 9.93 (s, 1H), 7.48 (dt, J=10.4, 2.2 Hz, 1H), 7.41 (d, J=1.8 Hz, 1H), 7.34 (ddd, J=8.7, 2.5, 1.3 Hz, 1H), 6.56 (d, J=2.3 Hz, 1H), 6.48-6.41 (m, 2H), 4.29 (q, J=7.1 Hz, 2H), 3.65 (q, J=7.0 Hz, 2H), 3.61 (s, 3H), 3.11 (s, 3H), 1.31 (t, J=7.1 Hz, 3H), 1.21 (t, J=7.0 Hz, 3H). ¹³C NMR (126 MHz, CDCl₃) δ 164.90, 164.87, 163.39, 161.44, 156.69, 149.53, 148.91, 141.94, 138.74, 138.65, 135.66, 132.58, 132.51, 131.98, 131.91, 130.18, 118.73, 115.81, 115.79, 112.24, 112.06, 111.80, 111.05, 111.02, 110.80, 64.16, 61.47, 55.79, 38.84, 14.40, 14.27. Molecular formula: C₂₄H₂₄ClFN₂O₇S₂. Molecular weight: 571.03.

Ethyl 3-bromo-5-(5-chloro-3-(N-(4-ethoxy-3-methoxyphenyl)-N-methylsulfamoyl)thiophene-2-carboxamido)benzoate (GR1-054). Reactant in final step (d): ethyl 3-amino-5-bromobenzoate. ¹H NMR (500 MHz, CDCl₃) δ 9.90 (s, 1H), 7.77 (d, J=1.7 Hz, 2H), 7.65-7.60 (m, 1H), 6.56 (d, J=2.1 Hz, 1H), 6.48-6.40 (m, 2H), 4.28 (q, J=7.1 Hz, 2H), 3.65 (q, J=7.0 Hz, 2H), 3.62 (s, 3H), 3.11 (s, 3H), 1.31 (t, J=7.1 Hz, 3H), 1.22 (t, J=7.0 Hz, 3H). 13C NMR (126 MHz, CDCl₃) δ 164.69, 156.64, 149.58, 148.95, 141.90, 138.50, 135.72, 132.49, 132.01, 131.93, 130.20, 128.26, 125.93, 122.40, 118.73, 118.69, 111.84, 111.15, 64.26, 61.52, 55.94, 38.85, 14.55, 14.29. Molecular formula: C₂₄H₂₄BrClN₂O₇S₂. Molecular weight: 631.93.

5-Chloro-N-(3-chloro-5-(1,3-dioxolan-2-yl)phenyl)-3-(N-(4-ethoxy-3-methoxyphenyl)-N-methylsulfamoyl)thiophene-2-carboxamide (GR1-058). Reactant in final step (d): 3-chloro-5-(1,3-dioxolan-2-yl)aniline. ¹H NMR (500 MHz, CDCl₃) 9.90 (s, 1H), 7.43 (t, J=2.0 Hz, 1H), 7.14 (dt, J=13.8, 1.6 Hz, 2H), 6.54 (p, J=3.0 Hz, 2H), 6.48 (d, J=9.2 Hz, 1H), 5.68 (s, 1H), 4.06-3.93 (m, 4H), 3.70 (t, J=7.0 Hz, 2H), 3.67 (s, 3H), 3.15 (s, 3H), 1.27 (t, J=7.0 Hz, 3H). ¹³C NMR (126 MHz, CDCl₃) δ 156.46, 149.66, 149.03, 142.35, 140.61, 138.26, 135.44, 134.50, 131.89, 130.12, 122.50, 119.88, 119.22, 115.65, 112.21, 110.85, 102.47, 65.26, 64.34, 56.00, 38.89, 14.53. Molecular formula: C₂₄H₂₄C₁₂N₂O₇S₂. Molecular weight: 587.48.

5-Chloro-N-(3-chloro-5-nitrophenyl)-3-(N-(4-ethoxy-3-methoxyphenyl)-N-methylsulfamoyl)thiophene-2-carboxamide (GR1-064). Reactant in final step (d): 3-chloro-5-nitroaniline. ¹H NMR (500 MHz, CDCl₃) δ 10.00 (s, 1H), 7.85 (t, J=2.1 Hz, 1H), 7.77 (t, J=2.0 Hz, 1H), 7.58 (t, J=2.0 Hz, 1H), 6.56 (d, J=2.2 Hz, 1H), 6.42-6.35 (m, 2H), 3.61 (q, J=7.0 Hz, 2H), 3.58 (s, 3H), 3.07 (s, 3H), 1.15 (t, J=7.0 Hz, 3H). ¹³C NMR (126 MHz, CDCl₃) δ 156.96, 149.61, 148.97, 148.68, 140.95, 139.04, 136.35, 135.42, 132.49, 131.94, 130.34, 124.31, 119.11, 118.49, 112.21, 111.64, 111.35, 64.24, 55.84, 38.86, 14.43. Molecular formula: C₂₁H₁₉Cl₂N₃O₇S₂. Molecular weight: 560.41.

5-Chloro-N-(5-chlorobenzo[d]thiazol-2-yl)-3-(N-(4-ethoxy-3-methoxyphenyl)-N-methylsulfamoyl)thiophene-2-carboxamide (GR1-066). Reactant in final step (d): 5-chlorobenzo[d]thiazol-2-amine. ¹H NMR (500 MHz, CDCl₃) δ 11.11 (s, 1H), 7.65 (d, J=2.0 Hz, 1H), 7.53 (d, J=8.4 Hz, 1H), 7.13 (dd, J=8.4, 2.0 Hz, 1H), 6.53 (d, J=2.5 Hz, 1H), 6.39 (dd, J=8.6, 2.5 Hz, 1H), 6.31 (d, J=8.6 Hz, 1H), 3.64 (s, 3H), 3.35 (q, J=7.0 Hz, 2H), 3.08 (s, 3H), 0.95 (t, J=7.0 Hz, 3H). ¹³C NMR (126 MHz, CDCl₃) δ 157.56, 156.74, 149.93, 149.69, 148.84, 138.59, 137.31, 133.20, 132.18, 131.55, 130.78, 130.33, 124.46, 121.76, 121.30, 118.67, 111.77, 110.81, 63.98, 55.74, 38.74, 14.40. Molecular formula: C₂₂H₁₉Cl₂N₃O₅S₃. Molecular weight: 572.49.

### Synthesis of GRZ-015 and GRZ-017 (Scheme 4).

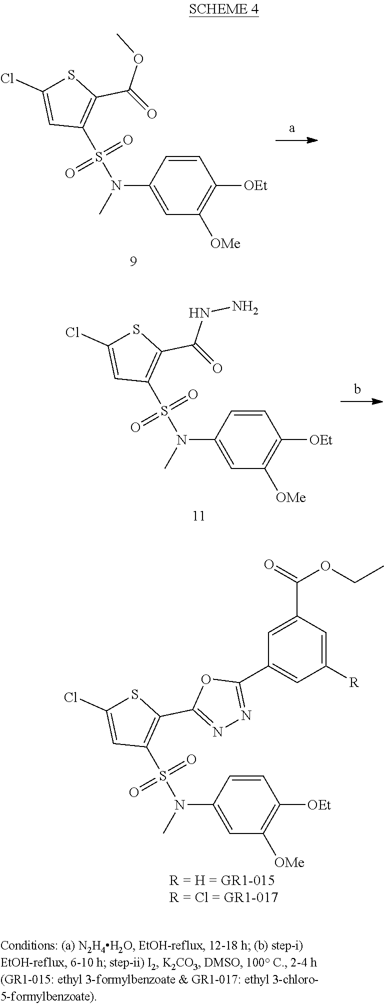

5-Chloro-N-(4-ethoxy-3-methoxyphenyl)-2-(hydrazinecarbonyl)-N-methylthiophene-3-sulfonamide (11). Thiophene-2-carboxylate (9, 1 mmol) in EtOH was added hydrazine monohydrate (4 mmol). The reaction mixture was refluxed overnight and cooled to room temperature. Evaporated the EtOH solvent and purified by column chromatography to get the title compound with 91% yield.

¹H NMR (500 MHz, CDCl₃) δ 9.16 (s, 1H), 6.90 (d, J=8.6 Hz, 1H), 6.79 (d, J=2.6 Hz, 1H), 6.70 (dd, J=8.6, 2.5 Hz, 1H), 4.19 (q, J=7.0 Hz, 2H), 3.94 (s, 3H), 3.30 (s, 3H), 1.56 (t, J=7.0 Hz, 3H). ¹³C NMR (126 MHz, CDCl₃) δ 159.18, 149.55, 148.54, 139.76, 134.79, 132.49, 131.97, 129.63, 119.18, 112.27, 111.25, 64.58, 56.13, 38.81, 29.68, 14.64. Molecular formula: C₁₅H₁₀ClN₃O₅S₂. Molecular weight: 419.895.

General Procedure for the Synthesis of 2,5-Disubstituted-1,3,4-oxadiazoles. A solution of aldehydes (1.0 mmol) and hydrazine 11 (1.0 mmol) in EtOH (10 mL) was refluxed until the condensation was complete (monitored by TLC, 3-4 h), and then the solvent was evaporated under reduced pressure, and the resulting residue was redissolved in DMSO (5 mL), followed by addition of potassium carbonate (3 mmol), iodine (1.2 mmol) in sequence. The reaction mixture was stirred at 100° C. until the conversion was complete (monitored by TLC, 1-4 h). After being cooled to room temperature, it was treated with 5% Na₂S₂O₃ (20 mL), extracted with EA (10 mL×3). The combined organic layer was washed with brine (10 mL×1), dried over anhydrous sodium sulfate, and concentrated. The given residue was purified through silica gel column chromatography using a mixture of ethyl acetate (EA) and petroleum ether (PE) as eluent to afford the desired oxadiazoles GR1-015 & GR1-017.

Ethyl 3-(5-(5-chloro-3-(N-(4-ethoxy-3-methoxyphenyl)-N-methylsulfamoyl)thiophen-2-yl)-1,3,4-oxadiazol-2-yl)benzoate (GR1-015). Reactant in final step (b): ethyl 3-formylbenzoate. ¹H NMR (500 MHz, CDCl₃) δ 8.64 (t, J=1.8 Hz, 1H), 8.21 (dt, J=7.9, 1.5 Hz, 1H), 8.02 (dt, J=7.8, 1.5 Hz, 1H), 7.57 (t, J=7.8 Hz, 1H), 6.64 (d, J=2.5 Hz, 1H), 6.57 (dd, J=8.5, 2.5 Hz, 1H), 6.47 (d, J=8.6 Hz, 1H), 4.42 (q, J=7.1 Hz, 2H), 3.77 (q, J=7.0 Hz, 2H), 3.67 (s, 3H), 3.23 (s, 3H), 1.42 (t, J=7.1 Hz, 3H), 1.31 (t, J=7.0 Hz, 3H). ¹³C NMR (126 MHz, CDCl₃) δ 165.35, 165.02, 157.71, 149.04, 148.07, 137.21, 134.36, 132.98, 132.85, 131.53, 131.07, 130.00, 129.16, 128.21, 124.37, 123.22, 118.64, 111.66, 111.03, 64.14, 61.51, 55.81, 39.01, 14.56, 14.32. Molecular formula: C₂₅H₂₄ClN₃OS₂, Molecular weight: 578.05.

Ethyl 3-chloro-5-(5-(5-chloro-3-(N-(4-ethoxy-3-methoxyphenyl)-N-methylsulfamoyl)thiophen-2-yl)-1,3,4-oxadiazol-2-yl)benzoate (GR1-017). Reactant in final step (b): ethyl 3-chloro-5-formylbenzoate. ¹H NMR (500 MHz, CDCl₃) δ 8.49 (t, J=1.5 Hz, 1H), 8.17 (t, J=1.8 Hz, 1H)), 7.99 (t, J=1.8 Hz, 1H), 7.29 (s, 1H), 6.65 (d, J=2.5 Hz, 1H), 6.53 (dd, J=8.5, 2.5 Hz, 1H), 6.45 (d, J=8.6 Hz, 1H), 4.42 (q, J=7.1 Hz, 2H), 3.78 (q, J=7.0 Hz, 2H), 3.67 (s, 3H), 3.21 (s, 3H), 1.42 (t, J=7.1 Hz, 3H), 1.32 (t, J=7.0 Hz, 4H). ¹³C NMR (126 MHz, CDCl₃) δ 164.21, 163.91, 158.07, 149.00, 148.03, 137.43, 135.48, 134.66, 133.14, 132.84, 130.71, 130.21, 126.23, 124.67, 123.95, 118.24, 111.47, 111.16, 64.15, 61.94, 55.90, 38.92, 29.69, 14.56, 14.28. Molecular formula: C₂₅H₂₃C₁₂N₃O₇S₂. Molecular weight: 612.49.

### Synthesis of GRX-018 and GR1-059 (Scheme 4).

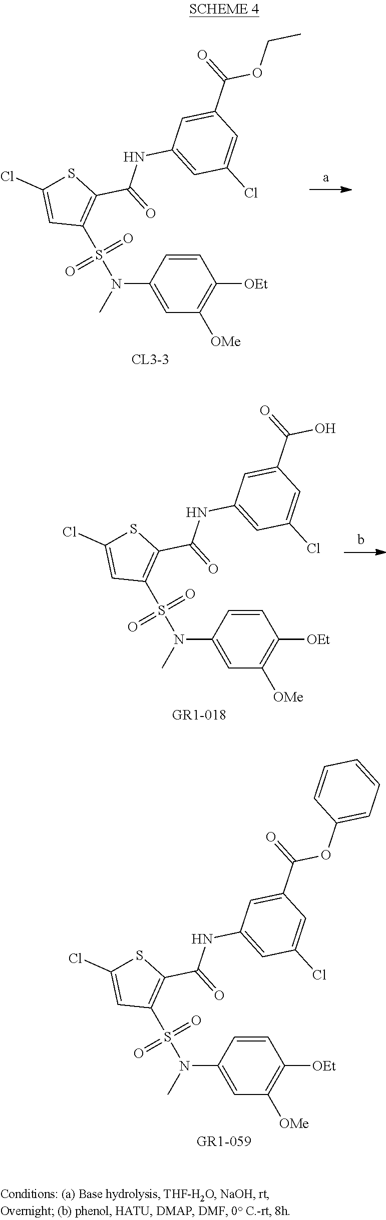 [SMILES: CCOC(=O)c1cc(Cl)cc(NC(=O)c2sc(Cl)cc2S(=O)(=O)N(C)c2ccc(OCC)c(OC)c2)c1 ; CCOc1ccc(N(C)S(=O)(=O)c2cc(Cl)sc2C(=O)Nc2cc(Cl)cc(C(=O)O)c2)cc1OC ; CCOc1ccc(N(C)S(=O)(=O)c2cc(Cl)sc2C(=O)Nc2cc(Cl)cc(C(=O)Oc3ccccc3)c2)cc1OC]

3-chloro-5-(5-chloro-3-(N-(4-ethoxy-3-methoxyphenyl)-N-methylsulfamoyl)thiophene-2-carboxamido)benzoic acid (GR1-018). Base hydrolysis of CL3-3. ¹H NMR (500 MHz, CDCl₃) δ 9.92 (s, 1H), 7.68 (dt, J=4.9, 1.9 Hz, 2H), 7.61 (t, J=1.7 Hz, 1H), 6.58 (d, J=2.2 Hz, 1H), 6.46-6.40 (m, 2H), 3.63 (d, J=6.0 Hz, 5H), 3.09 (s, 3H), 1.20 (t, J=7.0 Hz, 3H). ¹³C NMR (126 MHz, CDCl₃) δ 169.85, 156.76, 149.54, 148.92, 141.68, 138.57, 135.85, 134.91, 132.15, 131.94, 130.91, 130.27, 125.82, 124.05, 118.81, 118.58, 111.69, 111.20, 64.22, 55.87, 38.85, 14.47. Molecular formula: C₂₂H₂₀Cl₂N₂O₇S₂. Molecular weight: 559.43.

General procedure for final step (b): To a solution of carboxylic acid (GR1-018, 1 mmol) and HATU (1.2 mmol) in anhydrous DMF was added DMAP (1.2 mmol) at 0° C. under a nitrogen atmosphere. The reaction mixture was stirred for 10 min, phenol (1.0 mmol) was added and the reaction was stirred at room temperature for an additional 8 h. The reaction mixture was filtered and directly injected to prep-HPLC to give title compounds in 80-90% yield.

Phenyl 3-chloro-5-(5-chloro-3-(N-(4-ethoxy-3-methoxyphenyl)-N-methylsulfamoyl)thiophene-2-carboxamido)benzoate (GR1-059). Reactant in final step (d): phenol. ¹H NMR (500 MHz, CDCl₃) δ 9.95 (s, 1H), 7.76 (d, J=1.7 Hz, 1H), 7.70 (dt, J=8.4, 2.0 Hz, 2H), 7.34 (t, J=8.0 Hz, 2H), 7.21-7.16 (m, 1H), 7.13-7.09 (m, 2H), 6.58 (d, J=2.0 Hz, 1H), 6.45 (d, J=2.6 Hz, 2H), 3.66 (q, J=7.0 Hz, 2H), 3.61 (s, 3H), 3.10 (s, 3H), 1.22 (t, J=6.9 Hz, 3H). ¹³C NMR (126 MHz, CDCl₃) δ 163.46, 156.77, 150.71, 149.61, 148.96, 141.71, 138.64, 135.84, 134.94, 132.24, 131.99, 131.43, 130.26, 129.55, 126.13, 125.76, 123.82, 121.52, 118.75, 118.68, 111.79, 111.23, 64.25, 55.91, 38.85, 14.54. Molecular formula: C_{2B}H₂₄Cl₂N₂O₇S₂. Molecular weight: 635.52.

4-Acetylphenyl 3-chloro-5-(5-chloro-3-(N-(4-ethoxy-3-methoxyphenyl)-N-methylsulfamoyl)thiophene-2-carboxamido)benzoate (GR1-067). Reactant in final step (d): 1-(4-hydroxyphenyl)ethan-1-one. ¹H NMR (500 MHz, CDCl₃) δ 9.96 (s, 1H), 7.98-7.92 (m, 2H), 7.74 (dt, J=11.4, 1.7 Hz, 2H), 7.66 (t, J=2.0 Hz, 1H), 7.22 (d, J=2.0 Hz, 1H), 6.58 (d, J=2.2 Hz, 1H), 6.47-6.40 (m, 2H), 3.65 (q, J=7.0 Hz, 2H), 3.61 (s, 3H), 3.09 (s, 3H), 2.52 (s, 3H), 1.21 (t, J=7.0 Hz, 3H)). ¹³C NMR (126 MHz, CDCl₃) δ 196.75, 162.97, 156.81, 154.34, 149.59, 148.93, 141.56, 138.74, 135.93, 135.06, 135.03, 132.33, 132.01, 130.89, 130.28, 130.05, 125.77, 124.09, 121.78, 121.00, 118.80, 118.71, 111.74, 111.22, 64.21, 55.89, 38.85, 26.61, 14.53. Molecular formula: C₃₀H₂₆Cl₂N₂OS₂. Molecular weight: 677.56.

4-Benzoylphenyl 3-chloro-5-(5-chloro-3-(N-(4-ethoxy-3-methoxyphenyl)-N-methyl sulfamoyl)thiophene-2-carboxamido)benzoate (GR1-068). Reactant in final step (d): (4-hydroxyphenyl) (phenyl)methanone. ¹H NMR (500 MHz, CDCl₃) 9.97 (s, 1H), 7.83-7.78 (m, 2H), 7.76 (dt, J=11.4, 1.7 Hz, 2H), 7.73-7.69 (m, 2H), 7.66 (t, J=2.0 Hz, 1H), 7.52-7.47 (m, 1H), 7.40 (t, J=7.7 Hz, 2H), 7.27-7.24 (m, 2H), 6.58 (d, J=2.1 Hz, 1H), 6.48-6.41 (m, 2H), 3.66 (q, J=7.0 Hz, 2H), 3.61 (s, 3H), 3.10 (s, 3H), 1.22 (t, J=7.0 Hz, 3H). ¹³C NMR (126 MHz, CDCl₃) δ 195.46, 163.03, 156.82, 153.87, 149.60, 148.94, 141.57, 138.75, 137.46, 135.92, 135.41, 135.04, 132.52, 132.33, 132.01, 131.76, 130.93, 130.28, 129.97, 128.37, 125.79, 124.10, 121.54, 118.82, 118.72, 111.76, 111.23, 64.22, 55.91, 38.86, 14.55. Molecular formula: C₃₅H₂₈Cl₂N₂O₈S₂. Molecular weight: 739.63.

4-(4-Hydroxybenzoyl)phenyl 3-chloro-5-(5-chloro-3-(N-(4-ethoxy-3-methoxyphenyl)-N-methylsulfamoyl)thiophene-2-carboxamido)benzoate (GR1-069). Reactant in final step (d) bis(4-hydroxyphenyl)methanone. ¹H NMR (500 MHz, CDCl₃) δ 9.97 (s, 1H), 7.79-7.74 (m, 3H), 7.74 (d, J=2.0 Hz, 1H), 7.70-7.66 (m, 2H), 7.64 (t, J=2.0 Hz, 1H), 7.24-7.21 (m, 2H), 6.83-6.77 (m, 2H), 6.58 (d, J=2.1 Hz, 1H), 6.47-6.41 (m, 2H), 5.98 (s, 1H), 3.65 (q, J=7.0 Hz, 2H), 3.61 (s, 3H), 3.09 (s, 3H), 1.20 (t, J=7.0 Hz, 3H). ¹³C NMR (126 MHz, CDCl₃) δ 194.45, 163.14, 160.06, 156.88, 153.51, 149.56, 148.92, 141.49, 138.70, 136.01, 135.99, 135.05, 132.85, 132.36, 132.02, 131.45, 130.97, 130.31, 130.01, 125.86, 124.12, 121.46, 118.84, 118.74, 115.27, 111.76, 111.24, 64.26, 55.91, 38.87, 31.57, 22.63, 14.53, 14.09. Molecular formula: C₃₅H₂₈Cl₂N₂O₉S₂. Molecular weight: 755.63.

4-(4-(Prop-2-yn-1-yloxy)benzoyl)phenyl 3-chloro-5-(5-chloro-3-(N-(4-ethoxy-3-methoxyphenyl)-N-methylsulfamoyl)thiophene-2-carboxamido)benzoate (GR1-019). Reactant in final step (d): (4-hydroxyphenyl) (4-(prop-2-yn-1-yloxy)phenyl)methanone. ¹H NMR (500 MHz, CDCl₃) δ 9.96 (s, 1H), 7.79-7.72 (m, 6H), 7.67 (t, J=2.0 Hz, 1H), 7.26-7.22 (m, 3H), 6.98-6.93 (m, 2H), 6.58 (d, J=2.2 Hz, 1H), 6.47-6.39 (m, 2H), 4.68 (d, J=2.4 Hz, 2H), 3.66 (q, J=7.0 Hz, 2H), 3.61 (s, 3H), 3.09 (s, 3H), 2.46 (d, J=4.8 Hz, 1H), 1.22 (t, J=6.9 Hz, 3H). ¹³C NMR (126 MHz, CDCl₃) δ 194.20, 163.07, 161.11, 156.81, 153.55, 149.59, 148.94, 141.58, 138.74, 135.92, 135.03, 132.38, 132.31, 132.00, 131.45, 130.96, 130.80, 130.28, 125.79, 124.07, 121.46, 118.81, 118.71, 114.54, 111.74, 111.22, 77.76, 76.19, 64.22, 55.91, 38.85, 14.55. Molecular formula: C₃₈H₃₀Cl₂N₂O₉S₂. Molecular weight: 793.68.

### Example 3: Synthesis of Compounds Inactive in ABCA1-Luc Assay

Ethyl 3-(3-(N-(4-ethoxyphenyl)-N-methylsulfamoyl)benzo[b]thiophene-2-carboxamido)benzoate (CL1-47). Reagents in first step: methyl 3-(chlorosulfonyl)benzo[b]thiophene-2-carboxylate and 4-ethoxyaniline. Final step used ethyl 3-aminobenzoate. HPLC purity: 93%.

3-(3-(N-(4-ethoxyphenyl)-N-methylsulfamoyl)thiophene-2-carboxamido)benzoic acid (CL1-48). Synthesized from F420 via ester hydrolysis. HPLC purity: 94%.

3-(N-(4-ethoxyphenyl)-N-methylsulfamoyl)-N-(4-(trifluoromethyl)phenyl)thiophene-2-carboxamide (CL1-49). Synthesized via intermediate 3. Reagent in final step: 4-(trifluoromethyl)aniline. HPLC purity: 95%.

tert-Butyl 3-(3-(N-(4-ethoxyphenyl)-N-methylsulfamoyl)thiophene-2-carboxamido)benzoate (CL1-63). Synthesized via intermediate 3. Reagent in final step: tert-butyl 3-aminobenzoate. HPLC purity: 82%.

3-(N-(4-ethoxyphenyl)-N-methylsulfamoyl)-N-(3-(trifluoromethyl)phenyl)thiophene-2-carboxamide (CL1-64). Synthesized via intermediate 3. Reagent in final step: 3-(trifluoromethyl)aniline. HPLC purity: 84%.

3-(N-(4-ethoxyphenyl)-N-methylsulfamoyl)-N-(3-(methylcarbamoyl)phenyl)thiophene-2-carboxamide (CL1-84). Synthesized via intermediate 3. Reagent in final step: 3-amino-N-methylbenzamide. HPLC purity: 95%.

3-(N-(4-ethoxyphenyl)-N-methylsulfamoyl)-N-(3-(1,1,1,3,3,3-hexafluoro-2-hydroxypropan-2-yl)phenyl)thiophene-2-carboxamide (CL1-89). Synthesized via intermediate 3. Reagent in final step: 2-(3-aminophenyl)-1,1,1,3,3,3-hexafluoropropan-2-ol. HPLC purity: 96%.

N-(3-(2H-tetrazol-5-yl)phenyl)-3-(N-(4-ethoxyphenyl)-N-methylsulfamoyl)thiophene-2-carboxamide (CL1-96). Synthesized via intermediate 3. Reagent in final step: 3-(2H-tetrazol-5-yl)aniline. HPLC purity: 87%.

N-(5-acetyl-2-chlorophenyl)-3-(N-(4-ethoxyphenyl)-N-methylsulfamoyl)thiophene-2-carboxamide (CL1-116). Synthesized via intermediate 3. Reagent in final step: ethyl 3-amino-4-chlorobenzoate. HPLC purity: 99%.

3-(N-(4-ethoxyphenyl)-N-methylsulfamoyl)-N-(3-((2-methoxyethoxy)methyl)phenyl)thiophene-2-carboxamide (CL1-118). Synthesized via intermediate 3. Reagent in final step: 3-((2-methoxyethoxy)methyl)aniline. HPLC purity: 93%.

3-(N-(4-ethoxyphenyl)-N-methylsulfamoyl)-N-(3-(ethylcarbamoyl)phenyl)thiophene-2-carboxamide (CL1-121). Synthesized via intermediate 3. Reagent in final step: 3-amino-N-ethylbenzamide. HPLC purity: 92%.

N-(3-(diethylcarbamoyl)phenyl)-3-(N-(4-ethoxyphenyl)-N-methylsulfamoyl)thiophene-2-carboxamide (CL1-122). Synthesized via intermediate 3. Reagent in final step: 3-amino-N,N-diethylbenzamide. HPLC purity: 90%.

N-(3-(dimethylcarbamoyl)phenyl)-3-(N-(4-ethoxyphenyl)-N-methylsulfamoyl)thiophene-2-carboxamide (CL1-123). Synthesized via intermediate 3. Reagent in final step: 3-amino-N,N-dimethylbenzamide. HPLC purity: 97%.

Ethyl 3-(3-(N-(4-ethoxyphenyl)-N-isopropylsulfamoyl)thiophene-2-carboxamido)benzoate (CL1-152). Synthesized via intermediate 3. Reagent in sulfonamide alkylation: 2-iodopropane. Final step used reagent ethyl 3-aminobenzoate. HPLC purity: 96%.

Ethyl 3-(3-(N-phenylsulfamoyl)thiophene-2-carboxamido)benzoate (CL2-4). Reagents in first step: aniline and 1. Final step used ethyl 3-aminobenzoate. HPLC purity: 89%.

Ethyl 3-(3-(N-(4-methoxyphenyl) sulfamoyl)thiophene-2-carboxamido)benzoate (CL2-19). Reagents in first step: 4-methoxyaniline and 1. Final step used reagent ethyl 3-aminobenzoate. HPLC purity: 98%.

Ethyl 3-(3-(N-(4-ethoxyphenyl) sulfamoyl)-4-methylthiophene-2-carboxamido)benzoate (CL2-22). Reagents in first step: 4-ethoxyaniline and methyl 3-(chlorosulfonyl)-4-methylthiophene-2-carboxylate. Final step used ethyl 3-aminobenzoate. HPLC purity: 94%.

Ethyl 3-(3-(N-methyl-N-(4-propylphenyl) sulfamoyl)thiophene-2-carboxamido)benzoate (CL2-23). Reagents in first step: 4-propylaniline and 1. Final step used ethyl 3-aminobenzoate. HPLC purity: 95%.

Ethyl 3-(3-(N-(4-chlorophenyl)-N-methylsulfamoyl)thiophene-2-carboxamido)benzoate (CL2-37). Reagents in first step: 4-chloroaniline and 1. Final step used ethyl 3-aminobenzoate. HPLC purity: 95%.

Ethyl 3-(3-(N-methyl-N-(4-isopropoxyphenyl) sulfamoyl)thiophene-2-carboxamido)benzoate (CL2-44). Reagents in first step: 4-isopropoxyaniline and 1. Final step used ethyl 3-aminobenzoate. HPLC purity: 94%.

Ethyl 3-(3-(N-(2,2-dimethylbenzo[d][1,3]dioxol-5-yl)-N-methylsulfamoyl)-5-chlorothiophene-2-carboxamido)benzoate (CL2-66). Reagents in first step: 2,2-dimethylbenzo[d][1,3]dioxol-5-amine and 5. Final step used ethyl 3-aminobenzoate. HPLC purity: 91%.

Ethyl 3-(3-(N-(benzo[d][1,3]dioxol-5-yl)-N-methylsulfamoyl)-5-chlorothiophene-2-carboxamido)benzoate (CL2-70). Reagents in first step: benzo[d][1,3]dioxol-5-amine and 5. Final step used ethyl 3-aminobenzoate. HPLC purity: 86%.

3-(5-Chloro-3-(N-(3,4-diethoxyphenyl)-N-methylsulfamoyl)thiophene-2-carboxamido)benzoic acid (CL2-129). Synthesized from CL2-57 via ester hydrolysis. HPLC purity: 93%.

Ethyl 3-(5-chloro-3-(N-(3,4-diethoxyphenethyl)-N-methylsulfamoyl)thiophene-2-carboxamido)benzoate (CL2-144). Reagents in first step: 2-(4-ethoxyphenyl)ethan-1-amine and 5. Final step used ethyl 3-aminobenzoate. HPLC purity: 95%.

Diethyl 5-(5-chloro-3-(N-(3,4-diethoxyphenyl)-N-methylsulfamoyl)thiophene-2-carboxamido)isophthalate (CL2-145). Synthesized via intermediate 6. Reagent in final step: diethyl 5-aminoisophthalate. HPLC purity: 97%.

Ethyl 3-(5-chloro-3-(N-methyl-N-(1-(2,2,2-trifluoroethyl) azetidin-3-yl) sulfamoyl)thiophene-2-carboxamido)benzoate (CL2-161). Reagents in first step: 1-(2,2,2-trifluoroethyl)azetidin-3-amine and 5. Final step used ethyl 3-aminobenzoate. HPLC purity: 94%.

Ethyl 3-(3-(N-(1-acetylazetidin-3-yl)-N-methylsulfamoyl)-5-chlorothiophene-2-carboxamido)benzoate (CL2-163). Reagents in first step: 1-(3-aminoazetidin-1-yl)ethan-1-one and 5. Final step used ethyl 3-aminobenzoate. HPLC purity: 96%.

Ethyl 3-(5-chloro-3-(N-(3,4-diethoxyphenyl)-N-methylsulfamoyl)thiophene-2-carboxamido)-4-methylbenzoate (CL2-175). Synthesized via intermediate 6. Reagent in final step: ethyl 3-amino-4-methylbenzoate. HPLC purity: 91%.

Ethyl 5-(5-chloro-3-(N-(3,4-diethoxyphenyl)-N-methylsulfamoyl)thiophene-2-carboxamido)-2-methoxybenzoate (CL2-178). Synthesized via intermediate 6. Reagent in final step: ethyl 5-amino-2-methoxybenzoate. HPLC purity: 93%.

Ethyl 5-(5-chloro-3-(N-(2-ethoxypyrimidin-5-yl)-N-methylsulfamoyl)thiophene-2-carboxamido)benzoate (CL2-180). Reagents in first step: 2-ethoxypyrimidin-5-amine and 5. Final step used ethyl 3-aminobenzoate. HPLC purity: 92%.

Ethyl 3-(5-chloro-3-(N-methyl-N-(4-morpholinophenyl) sulfamoyl)thiophene-2-carboxamido)benzoate (CL2-194). Reagents in first step: 4-morpholinoaniline and 5. Final step used ethyl 3-aminobenzoate. HPLC purity: 93%.

Ethyl 2-(5-chloro-3-(N-(3,4-diethoxyphenyl)-N-methylsulfamoyl)thiophene-2-carboxamido)isonicotinate (CL3-1). Synthesized via intermediate 6. Reagent in final step: ethyl 2-aminoisonicotinate. HPLC purity: 92%.

### Example 4: Materials and Methods

High-Fat Diet and Drug Treatment. C57Bl/6 male mice (Jackson Laboratories) were placed on a high fat diet (HFD) (60% kcal from fat, Envigo) at 5 weeks. At 10 weeks, mice were acclimated to hydrogel replacement of drinking water (Clear H₂O) for 3 days. Then, for six weeks while HFD continued, mice received 10 mg/kg CL2-57 oral gavage Mon/Wed/Fri (vehicle: 9.5% PEG-200, 5% DMSO, 0.1% polysorbate 80 in water) plus 10 mg/kg/day CL2-57 in continuous hydrogel, or vehicle control. Mice were weighed weekly. For fast-refed studies, mice fasted for 16 hours, then refed for 4 hours. Blood glucose was measured with handheld glucometer. Sample size, chosen based on previous experience with glucose tolerance test, was ten mice per treatment. Mice were assigned to treatment groups by cage randomization upon arrival from Jackson Laboratories. Mice were assigned a unique ear tag number to enable blinded tissue analysis and data collection. Treatment and in vivo experimentation were not blinded. No animals were excluded from analysis, and no control for confounders such as cage location or treatment order was performed.

Plasma/Liver Triglycerides. Blood was collected in EDTA-coated tubes (Becton Dickinson) and centrifuged (3500 rpm, 15 minutes) to separate plasma. Livers were flash frozen in liquid nitrogen, then stored at −80° C. until homogenization in isopropanol. Triglyceride levels were measured using reagents from Wako Diagnostics per manufacturer's protocol.

Glucose Tolerance Test (GTT). After 16-hour fast, animals were administered 1.5 g/kg body weight (bwt) glucose by intraperitoneal (i.p.) injection. Glucose in tail vein blood was measured at 0, 10, 20, 30, 45, 60, and 120 minutes using a ONETOUCH® ULTRAMINI® glucometer (LifeScan, Inc). Additional blood was collected at multiple points between 0-30 minutes in heparinized capillary tubes and centrifuged, with plasma insulin levels determined by ELISA (ALPCO).

Neutrophil Counts. CD1 mice (n=5 per group) were administered T0901317, CL2-57, or vehicle at 10 mg/kg by oral gavage for three days. After final dose, blood samples were run on an Advia 120 analyzer (Bayer) for neutrophil counts.

Food Intake. C57Bl/6 mice (n=7, 2 male/5 female) were housed in wire-bottom cages. After three days of acclimatization, all mice received oral gavage vehicle for four days followed by 10 mg/kg CL2-57 for four days. Food mass in cage was weighed before first gavage and again before/after food replenishment on each subsequent day to determine daily food intake. Each mouse acted as its own control in this treatment paradigm.

Insulin Sensitivity Studies. Mice were injected with 1 U/kg bwt HUMALOG® insulin or vehicle (n=3 of each per treatment group) 15 minutes before sacrifice. Liver, subcutaneous fat, and gastrocnemius were collected and homogenized in lysis buffer (50 mM Tris-HCl (pH 7.4), 100 mM NaCl, 1 mM EDTA, 1 mM EGTA, 1% Triton™ X-100, with protease/phosphatase inhibitor (Complete, Roche)). Protein concentration was determined using Bradford reagent (Bio-Rad). Denatured protein (30 μg) was separated by SDS-PAGE (Mini-PROTEAN® TGX™ Gels 10%, Bio-Rad) and transferred to 0.45 μm nitrocellulose membranes. Membranes were blocked with 5% skim milk in TBST (1×TBS+0.1% polysorbate 20) for 1 hour, incubated with primary antibodies overnight at 4° C., washed with TBST, and incubated with horseradish peroxidase (HRP)-linked secondary antibodies for 1 hour. After TBST wash, Clarity™ Western ECL Substrate (Bio-Rad) was added. Blots were imaged and analyzed using a ChemiDoc™ MP and Image Lab Ver 6.0 (Bio-Rad). Protein levels were normalized to GAPDH (muscle) or β-actin (liver, adipose) for quantification. Primary antibodies: Cell Signaling Technology 4060 (pS473Akt, RRID:AB_2315049), 13038 (pT308Akt, RRID:AB_2629447), 4691 (pan-Akt, RRID:AB_915783), 5174 (GAPDH, RRID:AB 10622025) and Sigma A2228 (3-actin, RRID:AB_476697).

Luciferase. HepG2 cells (RRID:CVCL 0027) purchased from ATCC and used without further validation were stably transfected with pGreenFire1-LXRE-in-ABCA1 plasmid, which contains the ABCA1 promoter sequence linked to a luciferase response element, then selected over multiple generations with puromycin and frozen in liquid N₂. For assays, transfected cells were plated 24 hours in medium (Gibco) containing charcoal-stripped serum (Gemini), followed by a 24-hour treatment. Cells were lysed with passive lysis buffer (Promega), frozen at −80° C. for 15 minutes, and shaken at room temperature for 2 hours before quantification with luciferase assay system (Promega) on a Synergy Neo2 plate reader.

Cholesterol Efflux. Plated J774 cells (RRID:CVCL 0358) purchased from ATCC and used without further validation were incubated in serum-free medium containing 0.5 μM BODIPY-cholesterol (Cayman) for 24 hours. Medium was replaced with fresh serum-free medium containing test compound for 24 hours. For experiments with probucol (Sigma), it was added at 20 μM for the final 2 hours with test compound. Then, fresh serum-free medium containing 20 μg/mL purified recombinant apolipoprotein A1 (Sigma) was added for 6 hours. Acceptor-containing medium was transferred to a new plate, while cells were lysed with RIPA buffer. Fluorescence (Ex480/Em530) was measured in both components, with data reported as % efflux=(medium fluorescence)/(medium fluorescence+lysate fluorescence) normalized to background efflux (% efflux in DMSO-treated cells without exogenous apoA1).

qRT-PCR. For cell samples, treated cells were lysed with RLT plus lysis buffer (Qiagen). RNA was isolated with Qiagen RNEASY® Plus columns per manufacturer instructions, then reverse-transcribed to cDNA using SUPERSCRIPT® III Reverse Transcriptase (200 U/μL, Invitrogen) per supplier protocol. qPCR experiments were either performed with TAQMAN® gene expression master mix (Invitrogen) on a StepOne™ qPCR instrument or with PERFECTA® SYBR® Green SuperMix (Quanta Biosciences) on CFX Connect™ Real-Time PCR Detection System (BioRad), with fold-change quantified by ΔΔCₜ. For animal samples, 100 mg tissue was homogenized in 1 mL acid-guanidinium-phenol based reagent sold under the tradename TRIZOL® (Ambion), mixed vigorously in chloroform, and separated by centrifugation. Aqueous layer containing RNA was added to Qiagen RNEASY® Plus column; remaining stops were as described above for cell lysates.

qPCR primers. TaqMan primers (Mm=mouse, Hs=human; Thermo Fisher Scientific) and SYBR® green primers (all mouse) are provided in Tables 1 and 2, respectively.

TABLE 1

| Gene | Assay ID |
|---|---|
| ActB | Mm01205647_g1, Hs99999903_m1 |
| Gapdh | Hs02786624_g1 |
| Hprt | Mm03024075_m1 |
| Abca1 | Mm00442646_m1, Hs02059118_m1 |
| Abcg1 | Mm00437390_m1 |
| Abcg5 | Mm00446241_m1 |
| Ccl2 | Mm00441242_m1 |
| Cox2 | Mm03294838_g1 |
| Cxcl10 | Mm00445235_m1 |
| Cyp7a1 | Mm00484150_m1 |
| Il6 | Mm00446190_m1 |
| Irs1 | Mm01278327_m1 |
| Lpcat3 | Mm00520147_m1 |
| Nos2 | Mm00440502_m1 |
| Nr1h2 | Mm00437265_g1 |
| Nr1h3 | Mm00443451_m1 |
| Ppargc1a | Mm01208835_m1 |
| Slc2a1 | Mm00441480_m1 |
| Srebf1 | Mm00550338_m1, Hs01088691_m1 |
| Tnf | Mm00443258_m1 |

TABLE 2

| Gene | Primer | Sequence | SEQ ID NO: |
|---|---|---|---|
| 18S | forward | CTCAACACGGGAAACCTCAC | 1 |
| | reverse | AGACAAATCGCTCCAAC | 2 |
| Pck1 | forward | TGGAAGGTCGAATGTGTGGG | 3 |
| | reverse | AGCCCTTAAGTTGCCTTGGG | 4 |
| Ppara | forward | GTATCTCACCGGGAGGCGTT | 5 |
| | reverse | CAGAGCGCTAAGCTGTGATG | 6 |
| G6pase | forward | CCGGATCTACCTTGCTGCTC | 7 |
| | reverse | CACAGCAATGCCTGACAAGAC | 8 |
| Scd1 | forward | ATCGCCCCTACGACAAGAAC | 9 |
| | reverse | GTTGATGTGCCAGCGGTACT | 10 |

In vitro Immunoblot. Cells were lysed with RIPA buffer (Sigma) containing protease/phosphatase inhibitors (Cocktails II and III, respectively, EMD Millipore). Protein was quantified by BCA assay (Thermo Fisher Scientific). Denatured proteins were separated by gel electrophoresis (NuPage™ 4-12% Bis-Tris, Invitrogen) in MOPS running buffer at 120 V for 1 hour. Proteins were transferred to PDVF membranes (iBlot2). Membrane was blocked in 5% skim milk for 1 hour, incubated with primary antibody at 4° C. overnight, washed with TBST, and then incubated with HRP-linked secondary antibody (Cell Signaling Technology) for 1 hour at room temperature. After TBST wash, membrane was imaged using SUPERSIGNAL® West Femto™ substrate (Thermo Fisher Scientific) on Azure Biosystems c400 imager. Antibodies used: Abcam ab18180 (ABCA1, RRID:AB 444302), Thermo Fisher MA5-11685 (SREBP1, RRID:AB_10984077), Invitrogen MA5-15738 (GAPDH, RRID:AB_10977387).

siRNA Knockdown. J774 cells were plated in 24-well plates in serum-containing medium, to which an additional 100 μL of medium containing 10 pmol of siRNA and 3 μL of LIPOFECTAMINE® RNAIMAX® transfection reagent (Thermo Fisher Scientific) were added. After 24 hours, serum-free medium containing 10 μM C12-57 or vehicle control was added to each well for 24 hours. Cells were lysed and analyzed per qPCR and immunoblot procedures above. siRNAs used: Thermo Fisher 188584 (Nr1h3 silencer), 186947 (Nr1h2 silencer), and AM4611 (Silencer Negative Control siRNA #1).

Receptor Binding Assays. Nuclear hormone (NHR) assays were performed by DiscoverX (Fremont, CA) on their NHR-Scan platform of cell-based protein-protein interaction assays. This platform uses a 3-gal reporter that activates upon interaction of full-length receptor protein with steroid coactivator receptor peptides. In agonist mode, activity of CL2-57 at 10 μM was compared to maximal activity of published positive control agonists; in antagonist mode, reduction in activity with 10 μM CL2-57 from that observed at EC_{Bo} of published agonists was quantified.

TR-FRET Receptor Binding Assays. LanthaScreen” TR-FRET coactivator assay kits for LXRβ (Thermo Fisher Scientific) and PPARγ (Thermo Fisher Scientific) were performed according to manufacturer instructions using low-binding black 384-well plates and Synergy Neo2 plate reader. For the PPARγ assay, standardization was first performed with control agonist (pioglitazone), then run in two antagonist assays: dose-response of pioglitazone with or without a single concentration of CL2-57, and dose-response of CL2-57 cotreated with a single concentration (500 nM) of pioglitazone. Each compound dose was performed in quadruplicate on each plate, and each experiment was performed in triplicate.

Global Metabolomics. Liver and plasma samples were analyzed for global metabolomics profiling by Metabolon (Durham, NC) per their standard procedures, including extraction, detection via UPLC-MS/MS, quality control, and peak identification. Samples were blinded for analysis via bar code assignment before shipping to Metabolon. Principal components and hierarchical clustering analyses, visualization maps, and box-and-whisker plots were performed/produced by Metabolon.

Statistical Analysis. In vitro experimentation was performed in minimum of biological triplicate, with technical replicates included in each assay plate/gel. Specifically, each assay used 3 separate plates of cells across different days, and each plate included 2 technical replicates of the same treatment. Sample sizes for animal experiments are as indicated on each figure legend. Data visualization and statistical analysis (aside from metabolomics data) was performed in GraphPad Prism 8. p<0.05 was considered significant for all experiments; details of statistical tests for each experiment are provided in figure legends. All data were graphed as mean±S.D.

### Example 5: CL2-57 Selectively Induces ABCA1 and Lessens Inflammation In Vitro

Compound F420 was identified in a chemical library screen for selective inducers of ABCA1 versus SREBP1c (Aissa et al. (2021) ACS Pharmacol. Transl. Sci. 4(1):143-154). CL2-57 was synthesized and shown to exhibit enhanced ABCA1-linked luciferase activity in HepG2 liver cells (Eₘₐₓ=250% of vehicle; EC₅₀=1.8 μM). Increased ABCA1 mRNA and protein were measured in HepG2 hepatocarcinoma and J774 macrophage cell lines, with selectivity for Abca1 over Srebf1 induction demonstrated in HepG2 cells. In a fluorescent BODIPY-tagged cholesterol efflux assay (Sankaranarayanan et al. (2011) J. Lipid Res. 52(12):2332-40), CL2-57 dose-dependently increased cholesterol efflux from macrophages to apoA1, the primary acceptor of cholesterol from ABCA1 in vivo (Phillips (2013) J. Lipid Res. 54(8):2034-48), and increased efflux was blocked by ABCA1 inhibitor probucol (Wu et al. (2004) J. Biol. Chem. 279(29):30168-74). Stimulation of J774 cells with lipopolysaccharide increased TNFα and NOS2, modeling low-level inflammation that is chronically associated with T2D (Creely et al. (2007) Am. J. Physiol. Endocrinol. Metab. 292(3):E740-7; Kheder et al. (2016) Front. Cell Dev. Biol. 4:61); these increases were dose-dependently reversed by co-treatment with CL2-57.

### Example 6: CL2-57 Enhances Glucose Tolerance and Insulin Sensitivity in Mice Fed a High-Fat Diet

Male C57Bl/6 mice at five weeks were placed on a 60% kcal-high-fat diet for ten weeks and treated with vehicle or CL2-57 (n=10/group) from weeks 5-10, per the following regimen: 10 mg/kg/day hydrogel, plus 10 mg/kg oral gavage every Monday, Wednesday, and Friday. This regimen, which mimics an extended-release oral formulation, was chosen because of previous success in utilizing hydrogel formulation to ensure continuous drug exposure and minimize stress from gavage (Tai et al. (2014) J. Biol. Chem. 289(44):30538-55; Luo et al. (2015) BMC Neurosci. 16:67). Remaining hydrogel was measured to ensure consistent dosing.

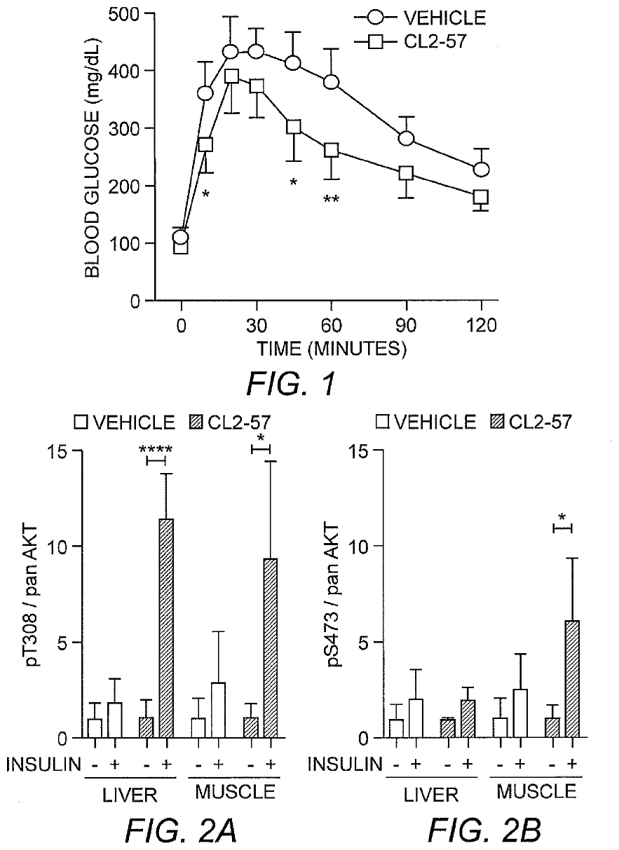

Little change in fasting/refed glucose and insulin levels was observed with treatment initially. After five weeks, however, mice treated with CL2-57 showed improved performance in a glucose tolerance test (GTT). Drug-treated mice exhibited lower blood glucose concentrations following a single i.p. glucose injection (FIG. 1), with total area under the curve during the 2-hour test reduced by 20%. These results were observed despite negligible change in insulin secretion during the first 20 minutes of the GTT.

To further investigate improvements in glucose handling, mice were administered i.p. insulin or vehicle 15 minutes before sacrifice (n=3/group). Excised muscle, liver, and adipose homogenates were probed for phosphorylated (S473 or T308) and pan-Akt protein. Increased phosphorylated:total Akt ratio in response to insulin correlates with tissue insulin sensitivity. Liver Akt phosphorylation at Thr308 increased with insulin in the CL2-57 group (FIG. 2A), while Ser473 did not change with insulin in either group (FIG. 2B). However, the strongest effect occurred in skeletal muscle, in which CL2-57 dramatically increased insulin sensitivity versus vehicle (FIG. 2A-2B). No treatment effect occurred in adipose where Akt phosphorylation responded to insulin in both groups.

### Example 7: Weight Gain and Adiposity Associated with HFD were Reduced by CL2-57

Treatment with CL2-57 reduced weight gain from 17% to 8% of baseline over six weeks. Treatment also shifted tissue mass from fat to lean as measured by TD-NMR analysis, a technique that uses changes in water relaxivity induced by the surrounding environment to quantify fat and lean tissue mass. By combining body weights with NMR data, it was determined that reduced weight gain was driven by reduced fat mass. On average, CL2-57-treated mice actually gained lean tissue mass, indicating that reduced weight gain was not driven by behavioral changes or compound toxicity, which was corroborated by follow-up food intake measurements. White adipose tissue mass in both visceral (renal) and superficial (inguinal) fat pads was reduced by >20% proportional to body weight with CL2-57, with no change in brown adipose or liver mass. Despite negligible change in liver size, liver triglycerides (TGs) were reduced by 35% with CL2-57 compared to vehicle control, which was mirrored by a 25% decrease in plasma TGs with CL2-57 compared to vehicle control.

These findings were further investigated at the transcriptional level in liver homogenates. These analyses showed that Abca1 expression increased with CL2-57 treatment along with ABCA1 protein compared to vehicle control; however, Abcg1 was unchanged. Bile acid transport gene Abcg5 was significantly reduced with CL2-57, and a diminishing trend was also observed with Cyp7-a1 mRNA, which encodes the rate-limiting step of bile acid synthesis (Zhang et al. (2009) Mol. Endocrinol. 23(2):137-45). A small increase in Srebf1 mRNA was observed, consistent with in vitro data, as well as a corresponding increase in the uncleaved, endoplasmic reticulum form of SREBP1c protein (Attie et al. (2001) J. Lipid Res. 42(11):1717-26). However, the mature, nuclear form of SREBP1c protein was unchanged with treatment, and several additional genes related to fatty acid synthesis and metabolism, such as Scd1 and Lpcat3 were significantly reduced by CL2-57 in HFD mice. Expression of mRNA encoding the nuclear receptor PPARα, a key regulator of fatty acid metabolism, likewise fell 75%. Finally, Pck1 and G6PC, which encode two key gluconeogenic enzymes, decreased by 70% and 73%, respectively, with CL2-57 treatment compared to the vehicle control treated group.

### Example 8: CL2-57 Modulates Gluconeogenesis and Fatty Acid Metabolism in HFD Mice

To fully characterize the non-lipogenic ABCA1 inducer lead and complement phenotypic data, global metabolomic profiling of mouse plasma and liver samples was performed. HFD produced a robust phenotype: >50% of all detected metabolites varied with diet (Table 3).

TABLE 3

| | | |
|---|---|---|
| Diet | Treatment | Interaction |

Plasma: 809 metabolites analyzed

| | | | |
|---|---|---|---|
| Biochemicals p < 0.05 | 469 | 61 | 115 |
| Biochemicals 0.05 < p < 0.10 | 64 | 51 | 70 |

Liver: 847 metabolites analyzed

| | | | |
|---|---|---|---|
| Biochemicals p < 0.05 | 400 | 51 | 162 |
| Biochemicals 0.05 < p < 0.10 | 62 | 44 | 73 |

Principal components and hierarchical clustering analyses illustrated this phenomenon, with close clustering by diet. Meanwhile, CL2-57 produced a broad treatment effect, impacting ˜20% of measured metabolites. Table 3 highlights the diet-dependence of this effect, with 14% (plasma) and 19% (liver) of metabolites varying with diet-treatment interaction compared to 8% and 6% with treatment alone. Further illustrating diet-dependence, C57Bl/6 control mice receiving CL2-57 via daily p.o. treatments for one week (10 mg/kg, n=4/group) showed no change in glucose or plasma free fatty acid metabolites, while low-magnitude increases in hepatic lipogenesis were observed (Table 4).

TABLE 4

| | |
|---|---|
| Metabolite | CL2-57/Vehicle |

Liver Glucose and Related Metabolites

| | |
|---|---|
| 1,5-Anhydroglucitol (1,5-AG) | 0.85 |
| Glucose | 0.96 |
| Glucose 6-phosphate | 0.71 |
| Fructose 6-phosphate | 1.13 |
| Fru/glu-1,6-diphosphate | 0.87 |
| 2,3-Diphosphoglycerate | 0.84 |
| Dihydroxyacetone phosphate | 0.75 |
| 2-Phosphoglycerate | 0.94 |
| 3-Phosphoglycerate | 0.84 |
| Phosphoenolpyruvate (PEP) | 0.90 |
| Pyruvate | 0.87 |
| Lactate | 0.94 |
| Glycerate | 1.06 |

Plasma Glucose and Related Metabolites

| | |
|---|---|
| 1,5-AG | 0.85^{†} |
| Glucose | 1.00 |
| 2,3-Diphosphoglycerate | 1.04 |
| 3-Phosphoglycerate | 0.95 |
| PEP | 1.16 |
| Pyruvate | 0.99 |
| Lactate | 0.88 |
| Glycerate | 1.07 |

Liver Free Fatty Acids

| | |
|---|---|
| Butyrate/isobutyrate (4:0) | 0.71 |
| Caproate (6:0) | 1.49 |
| Heptanoate (7:0) | 1.00 |
| Caprylate (8:0) | 1.28 |
| Caprate (10:0) | 1.18 |
| Myristate (14:0) | 2.67** |
| Pentadecanoate (15:0) | 2.93** |
| Palmitate (16:0) | 2.90** |
| Margarate (17:0) | 2.89** |
| Stearate (18:0) | 2.47** |
| Nonadecanoate (19:0) | 3.11** |
| Arachidate (20:0) | 3.08** |

Plasma Free Fatty Acids

| | |
|---|---|
| Butyrate/isobutyrate (4:0) | 1.32 |
| Caproate (6:0) | 1.12 |
| Heptanoate (7:0) | 1.12 |
| Caprylate (8:0) | 0.95 |
| Caprate (10:0) | 1.07 |
| Cis-4-decenoate (10:1n6) | 1.00 |
| (2 or 3) -Decenoate (10:1n7 or n8) | 1.16 |
| 10-Undecenoate (11:1n1) | 1.05 |
| Laurate (12:0) | 1.20 |
| 5-Dodecenoate (12:1n7) | 1.33 |
| Myristate (14:0) | 1.28 |
| Pentadecanoate (15:0) | 1.07 |
| Palmitate (16:0) | 1.15 |
| Margarate (17:0) | 1.10 |
| Stearate (18:0) | 1.23* |
| Nonadecanoate (19:0) | 1.22 |
| Arachidate (20:0) | 1.09 |

All data represented as ratio of CL2-57:veh mice, with n = 3-4 per group.
^{†}Increase, with p < 0.05.
*Decrease, with 0.05 < p < 0.10.
**Decrease, with p < 0.05.

In contrast, in HFD mice, metabolomics identified several pathways modulated by CL2-57. Liver cholesterol, reduced in HFD mice, was normalized to baseline with treatment via increased ABCA1 expression and, therefore, reverse cholesterol transport (RCT) (Sevastou et al. (2013) Biochim. Biophys. Acta 1831(1):42-60). HFD reduced liver cholesterol yet increased downstream oxysterol metabolites, 7-hydroxycholesterol and 4-cholesten-3-one, which again was reversed with CL2-57 treatment. Levels of cholesterol and these metabolites in plasma were unaffected by CL2-57.

Treatment with CL2-57 also reduced gluconeogenic metabolites. Increases of the four gluconeogenic precursors, in particular glutamine and glycerol, observed with HFD were attenuated by CL2-57 as compared to vehicle control. A similar trend of HFD-induced increases corrected by CL2-57 was observed for key gluconeogenic/glycolytic intermediates, including liver glucose.

These metabolomic data support the phenotypic readouts that were observed revealing improved glucose tolerance and insulin signaling and transcriptional data showing reduced gluconeogenic gene expression following CL2-57 treatment.

Finally, reduced adiposity and liver TG content of treated mice was corroborated by metabolomic profiling. HFD produced increases across nearly every lipid metabolite subclass in liver, with CL2-57 reversing many of these changes. These effects were particularly strong for free fatty acids (FFA), monoacylglycerols (MAG), and, to a lesser extent, diacylglycerols. Liver FFA levels in HFD mice ranged from 3-15× higher than normal diet mice; MAG increases with HFD versus normal diet ranged from 7-41x. CL2-57 reduced FFA concentrations from these increased levels by 40-90%; MAGs decreased 83-97% with CL2-57 compared to HFD alone. Plasma FFA and MAG levels were also increased by HFD, although only by <2-3× compared to normal diet. The effect of CL2-57 in reversing these increases was also muted in plasma (FFAs decreased 5-20%), indicating a primary treatment effect in the liver.

### Example 9: CL2-57 Broadly Attenuated Pro-Inflammatory Cytokines, Enzymes, and Metabolites in HFD Mice

Plasma concentrations of arachidonic acid (AA), the three fatty acid AA precursors, and three downstream eicosanoids were increased with HFD, with those increases attenuated by CL2-57. In particular, circulating AA increased 120% in HFD mice receiving vehicle, while HFD mice treated with CL2-57 experienced only a 37% increase over diet controls. Plasma levels of other proinflammatory lipid mediators, in particularly, lysophospholipids, followed a similar trend to AA metabolites. Increases up to 5× over normal diet were observed in HFD mice, along with 20-60% attenuation of those increases by CL2-57 treatment (Table 5).

TABLE 5

| | |
|---|---|
| HFD Veh/ | HED Drug/ |
| Chow Veh | Chow Veh |

Specialized Pro-Resolving Mediators

| | | |
|---|---|---|
| Eicosapentaenoate (EPA; 20:5n3) | 0.91 | 0.60^{†} |
| Docosapentaenoate (n3 DPA; 22:5n3) | 1.58* | 0.60^{†} |
| Docosahexaenoate (DPA; 22:6n3) | 1.63** | 0.76 |
| Docosapentaenoate (n6 DPA; 22:5n6) | 2.80** | 0.92 |

Lysophospholipids

| | | |
|---|---|---|
| 1-Palmitoyl-GPA (16:0) | 1.64** | 0.64^{††} |
| 1-Linoleoyl-GPA (18:2) | 1.66** | 0.57^{††} |
| 1-Arachidonoyl-GPA (20:4) | 2.67** | 0.65^{†} |
| 1-Palmitoyl-GPC (16:0) | 1.13 | 0.83^{†} |
| 2-Palmitoyl-GPC (16:0) | 1.18 | 0.81 |
| 1-Palmitoleoyl-GPC (16:1) | 0.74 | 0.81 |
| 2-Palmitoleoyl-GPC (16:1) | 0.56^{††} | 1.08 |
| 1-Stearoyl-GPC (18:0) | 1.30** | 0.94 |
| 1-Oleoyl-GPC (18:1) | 1.29 | 0.84 |
| 1-Linoleoyl-GPC (18:2) | 0.88 | 0.81^{†} |
| 1-Linolenoyl-GPC (18:3) | 0.76^{††} | 0.85 |
| 1-Arachidonoyl-GPC (20:4n6) | 1.73** | 0.73^{††} |
| 1-Lignoceroyl-GPC (24:0) | 1.00 | 0.99 |
| 1-Palmitoyl-GPE (16:0) | 0.84 | 0.95 |
| 1-Stearoyl-GPE (18:0) | 0.90 | 0.91 |
| 2-Stearoyl-GPE (18:0) | 1.44** | 0.74^{††} |
| 1-Oleoyl-GPE (18:1) | 1.44 | 0.79 |
| 1-Linoleoyl-GPE (18:2) | 1.03 | 0.81 |
| 1-Arachidonoyl-GPE (20:4n6) | 1.92** | 0.73^{††} |
| 1-Linoleoyl-GPS (18:2) | 5.16** | 0.39^{††} |
| 1-Palmitoyl-GPS (16:0) | 4.13** | 0.77 |
| 1-Oleoyl-GPS (18:1) | 4.94** | 0.62^{†} |
| 1-Linoleoyl-GPG (18:2) | 3.44** | 0.75^{†} |
| 1-Palmitoyl-GPI (16:0) | 0.96 | 0.56^{††} |
| 1-Stearoyl-GPI (18:0) | 1.03 | 0.82 |
| 1-Oleoy1-GPI (18:1) | 1.71* | 0.58^{†} |
| 1-Linoleoyl-GPI (18:2) | 0.84 | 0.72 |
| 1-Arachidonoyl-GPI (20:4) | 1.23 | 0.75^{†} |

Sphingolipids

| | | |
|---|---|---|
| Sphinganine | 1.96** | 0.67^{††} |
| Sphinganine-1-phosphate | 1.65** | 0.73 |
| Sphingadienine | 2.72** | 0.55^{††} |
| Sphingosine | 1.53** | 0.73 |
| Sphingosine-1-phosphate | 0.89 | 0.93 |

Middle column represents fold change with HFD vs. normal chow, while right column represents fold change with CL2-57 vs. vehicle in HFD mice.
^{†}Increase, with 0.05 < p < 0.10.
^{††}Increase, with p < 0.05.
*Decrease, with 0.05 < p < 0.10.
**Decrease, with p < 0.05. Data presented as mean for n = 4 per group.

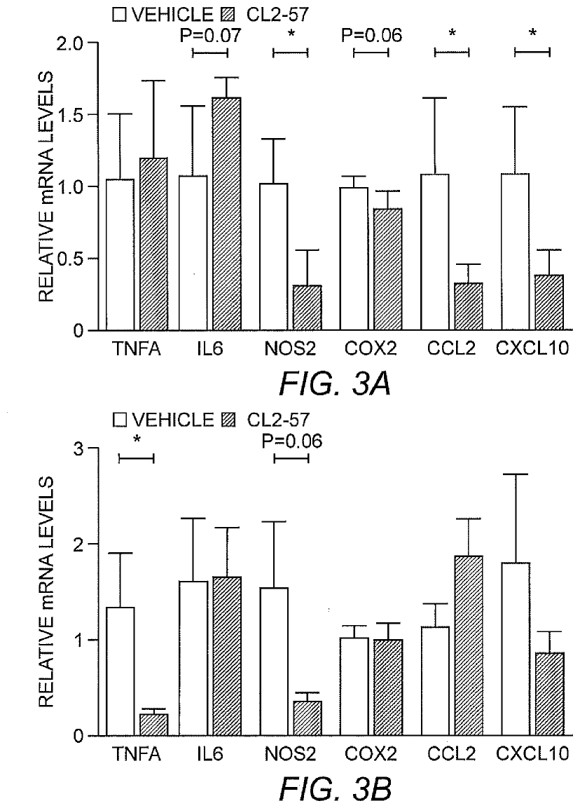

Liver and adipose were also profiled for proinflammatory enzymes (COX2 and NOS2), cytokines (TNFα and IL-6), and chemokines (CCL2 and CXCL10). Drug treatment substantially lowered mRNA levels of these markers, notably liver enzymes and chemokines (FIG. 3A) and adipose TNFα (FIG. 3B). Additionally, NO precursors arginine and guanidinosuccinate were increased by HFD, an effect reversed by CL2-57. The ketone body 3-hydroxybutyrate followed that same trend in plasma. Creatinine, urea, and bilirubin, which can serve as markers of global inflammation and/or organ toxicity, were unaffected by CL2-57.

### Example 10: Target Deconvolution Identified LXRβ-Selective Agonistic and PPAR/RXR Antagonistic Activity

Target identification with cell-based β-gal reporter assays revealed that CL2-57 functioned as a partial LXR agonist, with 2.5-fold β/α selectivity by Eₘₐₓ, as well as a weak antagonist at RXRγ, PPARβ/δ, and PPARγ, indicating that phenotypic screening could identify compounds engaging multiple targets. Dose-response binding studies measuring coactivator recruitment via TR-FRET confirmed LXRβ agonism with EC₅₀=1.3 μM, compared to 67 nM for T0901317. Weak competitive antagonism of pioglitazone at PPARγ was also observed with CL2-57. Addition of 10 μM CL2-57 caused a 2.1-fold right-shift in the TR-FRET dose-response for pioglitazone (p<0.01). At a constant pioglitazone concentration, CL2-57 again exhibited weak but significant antagonism (20-45% TR-FRET signal reduction) at concentrations ≥1 μM. The carboxylic acid derivative of CL2-57, which would be expected to form in vivo via ester hydrolysis, was inactive in all of the β-gal screens and in both TR-FRET binding assays. A summary of in vitro data highlights the selectivity of CL2-57 for LXRP/ABCA1 over LXRα/SREBP1c and its increasing efficacy versus T0901317 in assays of increasing biological complexity, from 75% in LXRβ binding to 183% in cholesterol efflux (Table 6).

TABLE 6

| | Activity of CL2-57 (10 μM) |
|---|---|
| Assay | as % of T0901317 (1 μM) |
| LXRα NHR screen | 34% |
| LXRβ NHR screen | 75% |
| ABCA1 luciferase | 127% |
| ABCA1 mRNA - HepG2 | 60% |
| SREBF1 mRNA - HepG2 | 39% |
| ABCA1 mRNA - J774 | 90% |
| ABCA1 protein - J774 | 132% |
| Cholesterol Efflux - J774 | 183% |

In similar analyses of other compounds disclosed herein low EC₅₀ were observed in an ABCA1 mRNA-HepG2 assay (Table 7).

TABLE 7

| Compound | EC₅₀ | |
|---|---|---|
| CL2-1 | >5 | μM |
| CL2-16 | 3.5 | μM |
| CL2-43 | 2.8 | μM |
| CL1-62 | >5 | μM |
| CL2-63 | 4.8 | μM |
| CL2-64 | >5 | μM |
| CL2-85 | 1.1 | μM |
| CL1-87 | >5 | μM |
| CL1-88 | 4.5 | μM |
| CL2-143 | 3.5 | μM |
| CL2-159 | >5 | μM |
| CL2-167 | 5 | μM |
| CL2-168 | 3.6 | μM |
| CL2-181 | 5 | μM |
| CL2-184 | 4.5 | μM |
| CL2-187 | 810 | nM |
| CL2-191 | >5 | μM |
| CL2-200 | 3.7 | μM |
| CL3-2 | >5 | μM |
| CL3-3 | 300 | nM |
| KB7-123 | 3.3 | μM |
| CL3-9 | 1.2 | μM |
| CL3-10 | 1.1 | μM |
| CL3-11 | 420 | nM |
| CL3-12 | 420 | nM |
| CL3-14/GR1-053 | 240 | nM |

In further analyses, low EC₅₀ were observed in an ABCA1 firefly reporter assay in CCF-001 cells (Table 8).

TABLE 8

| Compound | EC₅₀ | |
|---|---|---|
| CL1-39 | 2.527 | μM |
| CL1-87 | 3.967 | μM |
| CL2-01 | 4.645 | μM |
| CL2-16 | 2.356 | μM |
| CL2-43 | 2.041 | μM |
| CL2-57 | 0.817 | μM |
| CL2-63 | 12.55 | μM |
| CL2-85 | 0.190 | μM |
| CL2-143 | 1.619 | μM |
| CL2-159 | 5.393 | μM |
| CL2-167 | 2.190 | μM |
| CL2-168 | 1.362 | μM |
| CL2-181 | 4.989 | μM |
| CL2-184 | 4.948 | μM |
| CL2-187 | 0.247 | μM |
| CL2-191 | 8.415 | μM |
| CL3-09 | 0.5296 | μM |
| CL3-10 | 0.339 | μM |
| CL3-11 | 0.066 | μM |
| CL3-12 | 0.138 | μM |
| KB7-123 | 1.491 | μM |
| CL3-3 | 0.099 | μM |
| GR1-001 | 0.820 | μM |
| GR1-002 | 2.023 | μM |
| GR1-008 | 1.601 | μM |
| GR1-015 | 3.081 | μM |
| GR1-019 | 0.066 | μM |
| GR1-022 | 1.741 | μM |
| GR1-046 | 2.704 | μM |
| GR1-047 | 2.530 | μM |
| GR1-048 | 1.243 | μM |
| GR1-049 | 0.790 | μM |
| GR1-050 | 1.089 | μM |
| GR1-051 | 3.602 | μM |
| GR1-052 | 1.550 | μM |
| GR1-053 | 0.029 | μM |
| GR1-054 | 0.564 | μM |
| GR1-058 | 1.946 | μM |
| GR1-059 | 0.135 | μM |
| GR1-064 | 0.578 | μM |
| GR1-067 | 0.011 | μM |
| GR1-068 | 0.148 | μM |
| GR1-069 | 0.211 | μM |
| GW3965 | 0.269 | μM |
| T0901317 | 0.019 | μM |

The importance of LXR agonism to in vitro activity of CL2-57 was further interrogated. Experiments employing siRNA knockdown (efficiency by qPCR: 38% for LXRα and 36% for LXRβ) demonstrated that the effect of CL2-57 on ABCA1 induction in vitro was mediated by LXRβ. Induction of both Abcg1 and Slc2a1 (GLUT1) by CL2-57 was also reduced by LXRβ knockdown, while Irs1 increases were not affected by loss of either LXR isoform. Meanwhile, LXR target gene Apoe was not significantly affected by CL2-57 treatment in J774 cells.

Finally, ABCA1-linked luminescence increased (from 2.5- to 4.5-fold) with CL2-57 titration in the presence of RXR agonist bexarotene; however, no increase was observed when CL2-57 was titrated in the presence of pan-LXR agonist T0901317. Having detected LXR agonist activity with CL2-57, CD1 mice were treated with LXR agonists to evaluate for known adverse effects. Three daily p.o. treatments with CL2-57 did not affect plasma TG or cholesterol, nor circulating neutrophil levels (n=5/group), whereas T0901317 increased TG and reduced neutrophils. In a supplemental study, C57Bl/6 mice treated for one week with daily p.o. CL2-57 also experienced no changes in plasma TG or cholesterol compared to vehicle controls.

### Example 11: LXR Agonism in Mouse Models of Alzheimer's Disease

To evaluate preclinical efficacy, CL3-3 was studied in 5×FAD mice. 5×FAD mice, which overexpress five human mutations in APP and PSEN1, exhibit amyloid deposition, gliosis, and progressive neuronal loss accompanied by cognitive and motor deficiencies, recapitulating many of the features of human AD. Female 5×FAD mice (JAX) at 16 weeks of age were treated for 10 days with either CL3-3 (10 mg/kg PO gavage daily) or vehicle. Upon completion of treatment, mouse whole brain hemispheres were analyzed for mRNA expression levels of pro-inflammatory markers including CCL2, CCL3, CCL5 CXCL10, IL-6, COX2 and NOS2. Plasma from the same mice was analyzed. Notably, the level of each of the inflammatory markers was reduced in the brains of mice treated with CL3-3 (CCL2, COX2, NOS2, p<0.05 vs. vehicle; CCL5, p=0.08 vs. vehicle); however, circulating TG levels were not significantly altered.

A longer-term study was conducted in EFAD mice, which express the same five human AD mutations as 5×FAD mice and have also undergone targeted replacement of the mouse Apoe gene with one of the human APOE alleles: ε2, ε3, or ε4. In this study, EFAD mice with heterozygous ε3/ε4 alleles (E3/4FAD) were treated from 4 months of age until 6 months of age with CL3-3 (100 mg/kg/day in hydrogel replacement of drinking water) or vehicle.

Multiple readouts of AD-related pathology were conducted with subsets of these mice. Brain regions were first analyzed via ELISA for levels of soluble, oligomeric Aβ (oAβ), which is hypothesized to be the toxic form of amyloid that underlies downstream pathology and clinical presentation associated with AD. In E3/4FAD mice, oAβ levels decreased in the hippocampus in male mice and in the cerebral cortex in female mice. Treatment with CL3-3 also was synaptoprotective in cerebral cortex regions, particularly in male mice. Expression of synaptic proteins drebrin and PSD95 were increased in male E3/4FAD mice treated with CL3-3, as determined by western blot analysis.

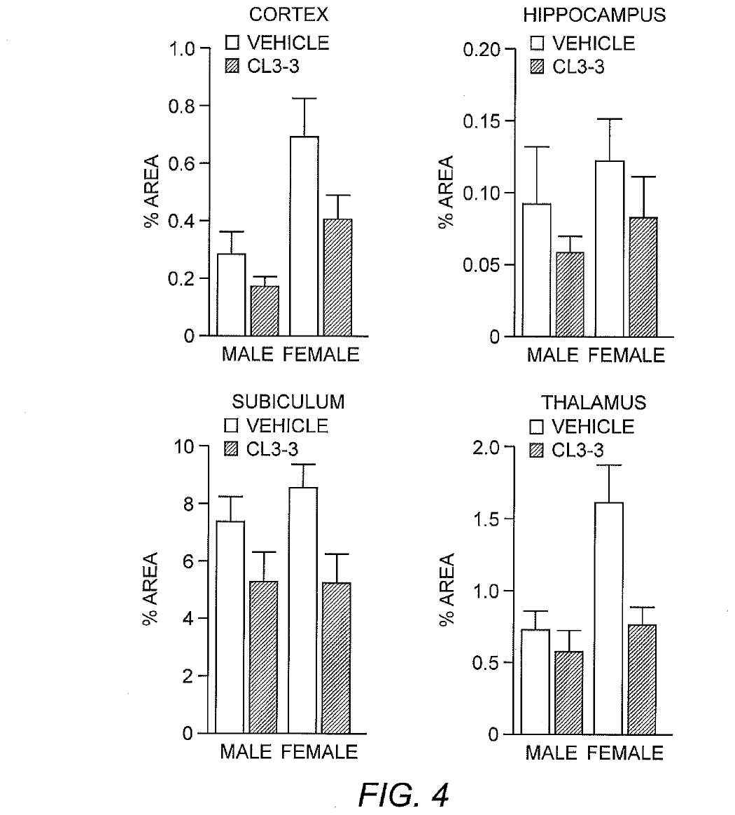

The remaining subset of brain hemispheres from the treated E3/4FAD mice were fixed and analyzed by staining with thioflavin S (Thio-S). Thio-S is a fluorescent dye that binds to abnormally folded protein aggregates (i.e., amyloid). In E3/4FAD mice, staining with this reagent demonstrated the amyloid plaque burden. Notably, treatment with CL3-3 decreased the % area stained by Thio-S across all measured brain regions and in both males and females (FIG. 4).

1: A compound having the structure of Formula (I), or a pharmaceutically acceptable salt or prodrug thereof: 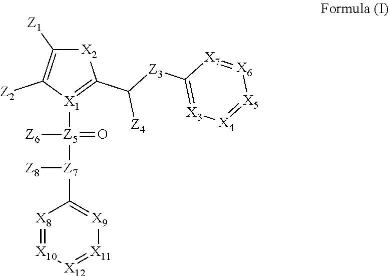
wherein,
X₁, X₂, X₃, X₄, X₅, X₆, X₇, X₈, X₉, X₁₀, and X₁₁ are each independently selected from the group of carbon (C), nitrogen (N), sulfur (S), oxygen (O), NH, or CY₁;
each occurrence of Y₁ is independently selected from the group consisting of hydrogen, alkyl, alkoxy, haloalkyl, hydroxyalkyl, hydroxy, cyano, halo, alkoxy, ketone, ester, carboxamide, sulfide, sulfoxide, sulfone, sulfonamide, a 5- or 6-membered monocyclic heteroaryl, and a 5- or 6-membered fused-ring;
Z₁, Z₂, Z₃, Z₄, Z₆, and Z₈ are each independently selected from the group of hydrogen, halo, O, S, N, NH, C(═O), CH₂, hydroxy, or CY₂;
Z₅ is S;
Z₇ is N or C, and
each occurrence of Y₂ is independently selected from the group consisting of hydrogen, alkyl, alkoxy, haloalkyl, hydroxyalkyl, hydroxy, cyano, and halo,
with the proviso that either Z₁ is not hydrogen, or Z₈ is alkyl when Z₇ is N.

2: The compound of claim 1, said compound having the structure of Formula (II), or a pharmaceutically acceptable salt or prodrug thereof: 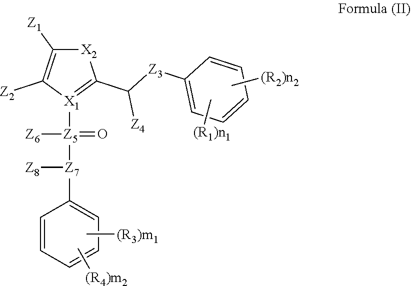
wherein,
X₁ and X₂ are each independently selected from the group of C, N, S, O, NH, or CY₁;
each occurrence of Y₁ is independently selected from the group consisting of hydrogen, alkyl, alkoxy, haloalkyl, hydroxyalkyl, hydroxy, cyano, and halo;
Z₁, Z₂, Z₃, Z₄, Z₆, and Zₑ are each independently selected from the group of hydrogen, halo, O, N, NH, C(═O), S, CH₂, hydroxy, or CY₂;
Z₅ is S;
Z₇ is N or C; and
each occurrence of Y₂ is independently selected from the group consisting of hydrogen, alkyl, alkoxy, haloalkyl, hydroxyalkyl, hydroxy, cyano, and halo;
m₁ and m₂ are independently 0, 1, 2, 3, or 4;
n₁ and n₂ are independently 0, 1, 2, 3, or 4; and
R₁, R₂, R₃, and R₄ are each independently selected from the group of hydrogen, halo, hydroxy, nitro, C₁-C₆ alkyl, alkoxy, ketone, ester, carboxamide, sulfide, sulfoxide, sulfone, sulfonamide, C₁-C₆ fluoroalkyl, cyano, —O(C₁-C₆ alkyl), and —O(C₁-C₆ fluoroalkyl),
with the proviso that either Z₁ is not hydrogen, or Z₈ is alkyl when Z₇ is N.

3: The compound of claim 1, said compound having the structure of Formula (III), or a pharmaceutically acceptable salt or prodrug thereof: 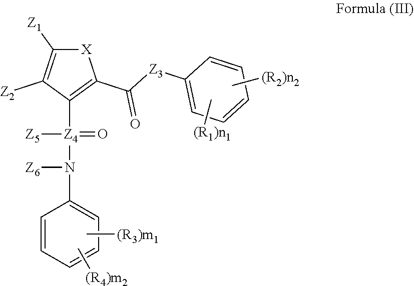
wherein,
X is S, O, or NH;
Z₁, Z₂, Z₃, Z₅, and Z₆ are each independently selected from the group of hydrogen, halo, O, S, N, NH, CH₂, hydroxy, or CY₁;
Z₄ is S;
each occurrence of Y₁ is independently selected from the group consisting of hydrogen, alkyl, alkoxy, haloalkyl, hydroxyalkyl, hydroxy, cyano, and halo;
m₁ and m₂ are independently 0, 1, 2, 3, or 4;
n₁ and n₂ are independently 0, 1, 2, 3, or 4; and
R₁, R₂, R₃, and R₄ are each independently selected from the group of hydrogen, halo, hydroxy, nitro, C₁-C₆ alkyl, alkoxy, ketone, ester, carboxamide, sulfide, sulfoxide, sulfone, sulfonamide, C₁-C₆ fluoroalkyl, cyano, —O(C₁-C₆ alkyl), and —O(C₁-C₆ fluoroalkyl),
with the proviso that either Z₁ is not hydrogen, or Z₆ is alkyl.

4-7. (canceled)

8: The compound of claim 1, said compound having the structure of Formula (VIII), or a pharmaceutically acceptable salt or prodrug thereof: 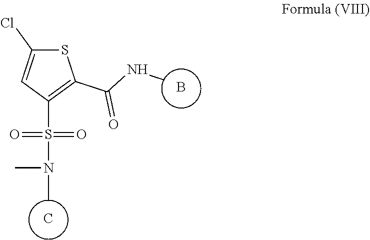
wherein,
Ring B is selected from 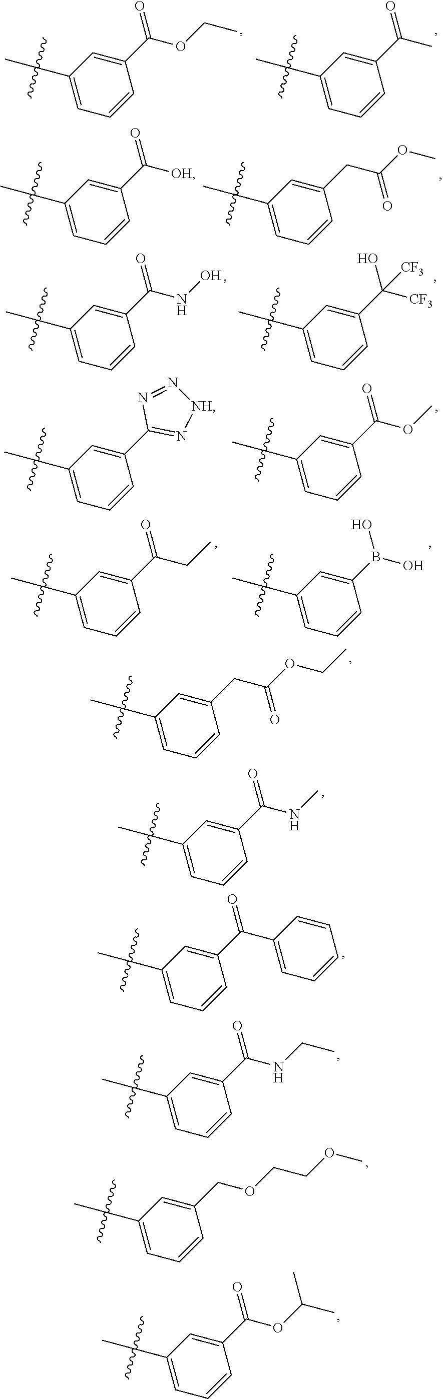 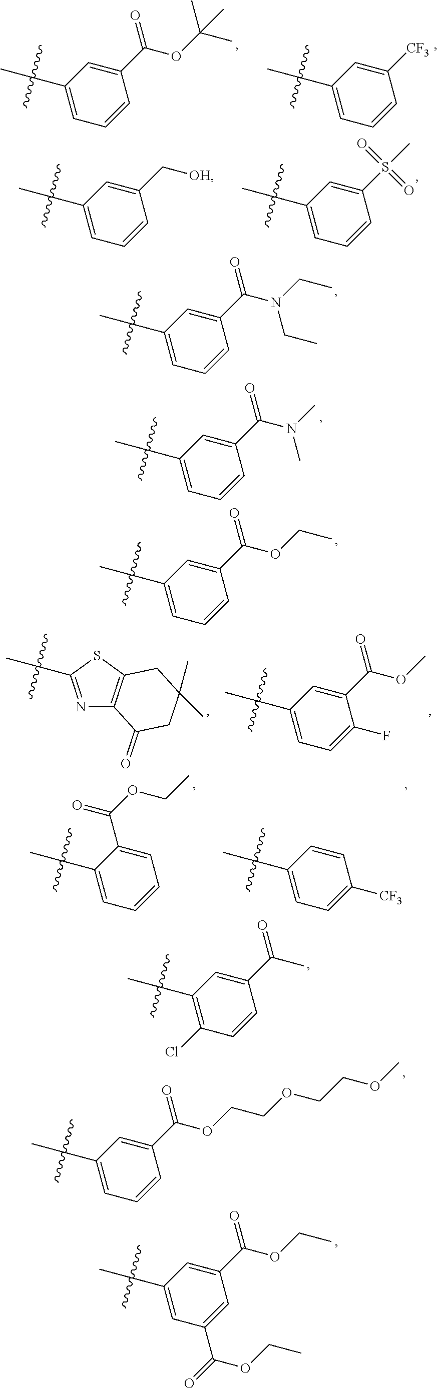 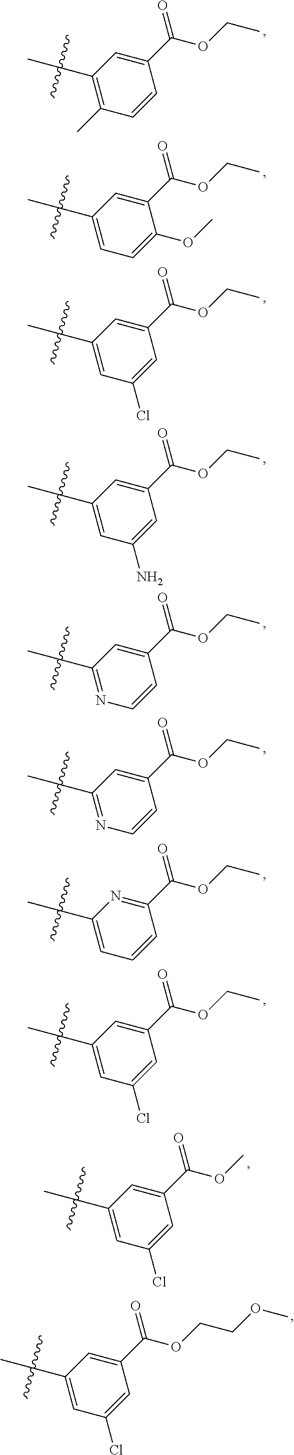 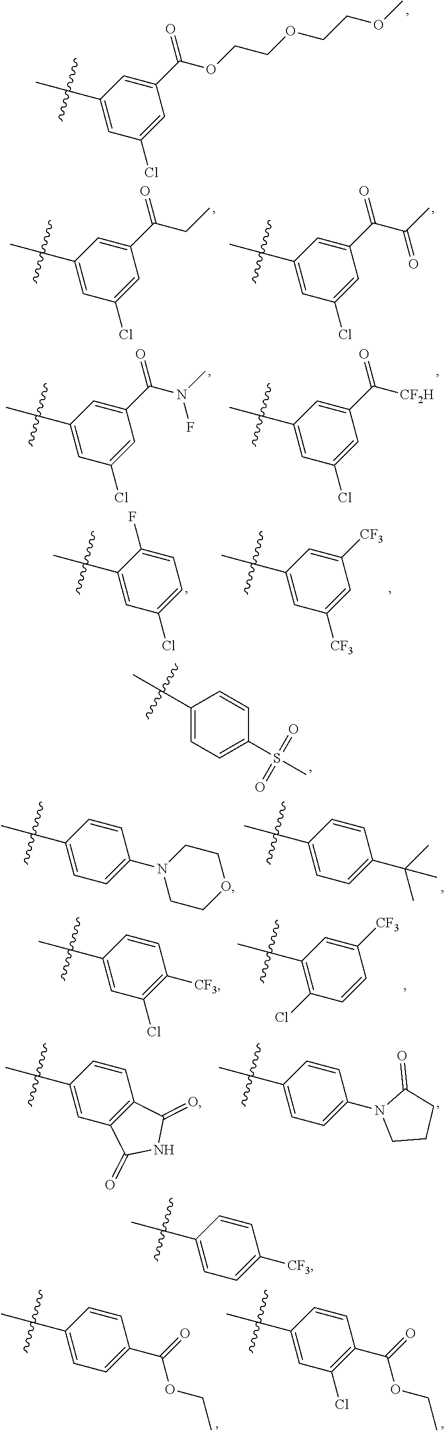 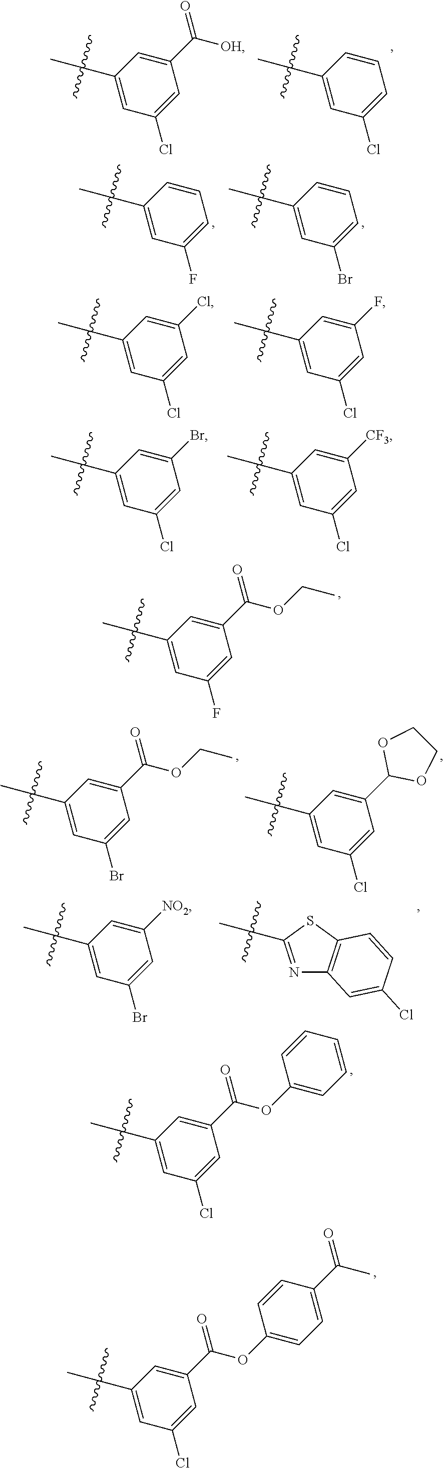 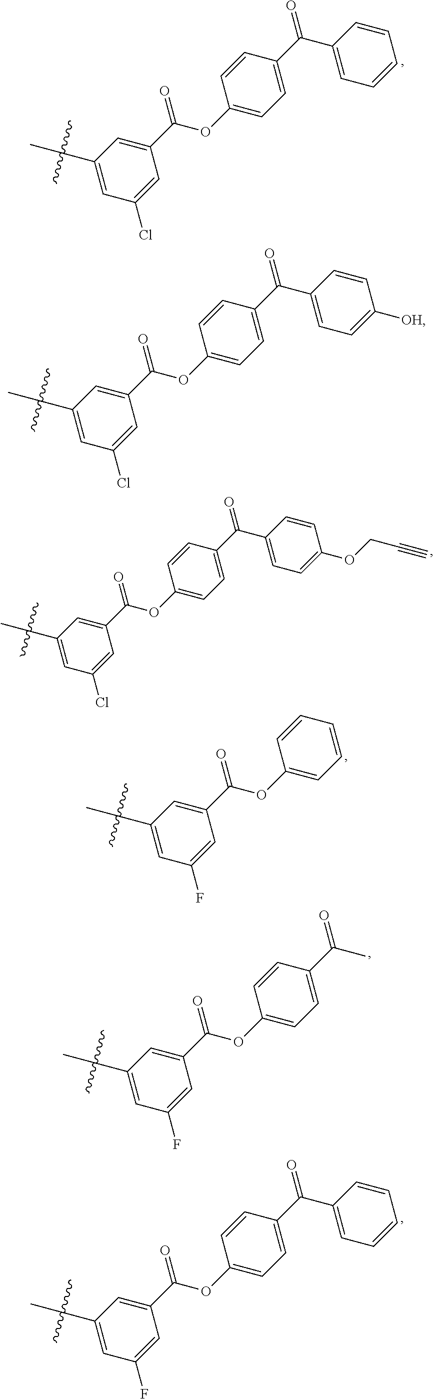 [SMILES: *c1cc(Cl)cc(C(=O)Oc2ccc(C(=O)c3ccccc3)cc2)c1 ; *c1cc(Cl)cc(C(=O)Oc2ccc(C(=O)c3ccc(O)cc3)cc2)c1 ; *c1cc(Cl)cc(C(=O)Oc2ccc(C(=O)c3ccc(OCC#C)cc3)cc2)c1 ; *c1cc(F)cc(C(=O)Oc2ccccc2)c1 ; *c1cc(F)cc(C(=O)Oc2ccc(C(C)=O)cc2)c1 ; *c1cc(F)cc(C(=O)Oc2ccc(C(=O)c3ccccc3)cc2)c1] 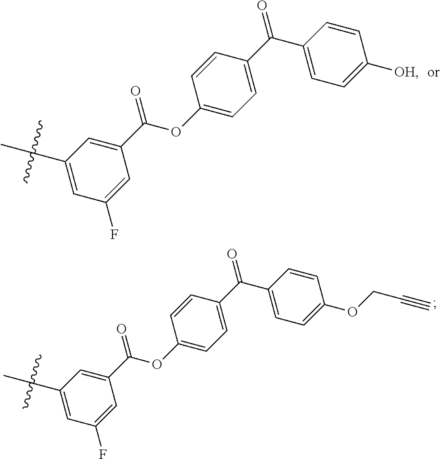 [SMILES: *c1cc(F)cc(C(=O)Oc2ccc(C(=O)c3ccc(O)cc3)cc2)c1 ; *c1cc(F)cc(C(=O)Oc2ccc(C(=O)c3ccc(OCC#C)cc3)cc2)c1]
and
Ring C is selected from

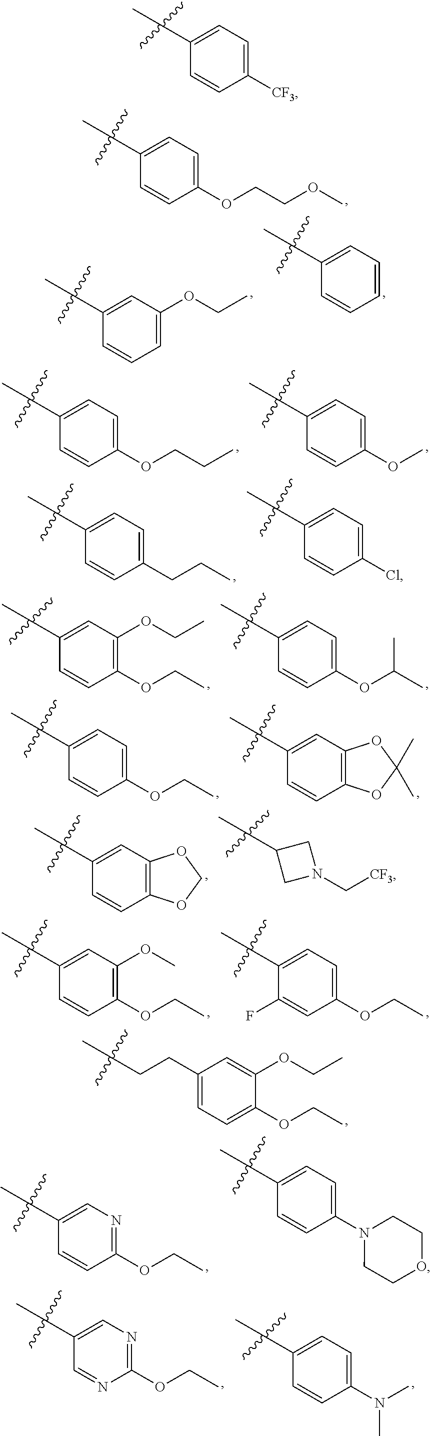

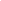

9-11. (canceled)

12: The compound of claim 1, said compound having the structure of:

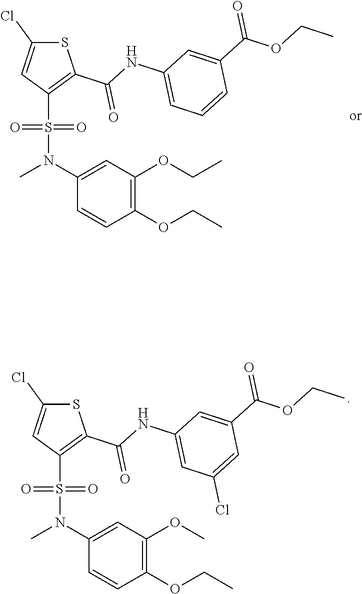 [SMILES: CCOC(=O)c1cccc(NC(=O)c2sc(Cl)cc2S(=O)(=O)N(C)c2ccc(OCC)c(OCC)c2)c1 ; CCOC(=O)c1cc(Cl)cc(NC(=O)c2sc(Cl)cc2S(=O)(=O)N(C)c2ccc(OCC)c(OC)c2)c1]

13: A pharmaceutical composition comprising a compound of claim 1 in admixture with a pharmaceutically acceptable carrier or vehicle.

14: A method for inducing ATP-binding cassette transporter A1 (ABCA1) expression without lipogenesis comprising administering to a subject in need of treatment an effective amount of a pharmaceutical composition of claim 13 thereby inducing ABCA1 expression in the subject without lipogenesis.

15: A method for treating or preventing type 2 diabetes or dementia comprising administering to a subject in need of treatment an effective amount of a pharmaceutical composition of claim 13 thereby treating or preventing the subject's type 2 diabetes or dementia.
